SEQUENCE LISTING (80 sequences)

SEQ ID NO: 1 - RNA, 7042 nt, Mus musculus
  source 1..7042: mol_type = mRNA
cttcagtccttcctcacactgccgccagcctgggaaccctgagagccggcagggaaggcctggcttctcactgcctgtagtgcctcccttacaaactcttgaactttgttctgtctagtgagagacctgcctctcctgccccagagctctgctgcctcaccatgaatggggtgagacgtgacctttggaggtgactataactgaagattgaattacagaagccaaaaaattaaataaataaaaggtttttgagtcatgatgcaagaatctgggtctgagacaaaaagtaacggatcagccatccagaacgggtccagcggtggcaaccacttactagagtgcggggcacttcgtgacactcggtccaacggagaggcaccagcggtggacctgggggcagccgaccttgcccacgtccagcagcagcaacagcagcagcaacaacagcagcagcagcaacagcagcagcagcagcagcaagttagtggattaaagtctcccaagaggaatgacaagcaaccagctcttcaggttcccgtgtcagtggctatgatgacacctcaggttatcactcctcaacaaatgcagcagatcctccagcagcaggtgctgagcccccagcagctccaggttctcctccagcagcagcaggccctcatgcttcaacagcagcttcaagaattttataaaaaacaacaggaacagttgcagcttcaacttctccaacagcaacatgctggaaaacagccgaaagagcagcaggtggctacccagcagttggctttccagcagcagcttctccagatgcagcagctgcagcagcagcacctcttgtctctccagcgccaaggcctcctaacaattcagccggggcagcctgcccttcccctccaacccctcgctcaaggcatgattccaacggaactgcagcagctctggaaagaagtgacaagtgcccacactgcagaggaaaccacaagcagcaaccacagcagcctagacctgaccagcacatgtgtctcgtcctcggcaccttccaagtcctccctaatcatgaacccgcatgcctctaccaatggacagctctcggtccacactcccaaaagggaaagcttgtcccacgaggagcacccccacagccaccctctctatggacatggcgtatgcaagtggccaggctgtgaggcggtttgtgacgacttcccagcctttctaaaacatctcaacagtgagcatgcgctggacgatagaagcacagctcaatgtagagtacaaatgcaggttgtacagcagttagagctacagcttgcaaaagacaaagagcgcctgcaagccatgatgacccacctgcatgtgaagtctacagaacccaaagctgcccctcagcccctgaatctggtatcaagtgtcaccctctccaagtctgcctcagaggcttctccacagagcttacctcatactccaacaacccccaccgcccccctgactcctgtcacccaaggcccctccgtcatcaccaccaccagcatgcacacggtgggacctatccgcaggcggtactcagacaaatacaacgtgcccatttcttcagcagatattgcgcagaaccaagaattttataagaacgcggaagttagaccaccatttacatatgcatctttaatcaggcaggccattctcgaatctccagaaaagcagctaacactaaacgaaatctataactggttcacacgaatgtttgcttacttccgacgcaatgcagccacgtggaagaatgcagtgcgtcataatcttagtctccacaagtgttttgtgcgagtagagaacgttaaaggggcagtatggacagtggatgaagtagagttccaaaaacggaggccacaaaagatcagtggtaacccttcccttattaaaaacatgcagagcagccacgcctactgcacacctctcaatgcagctttacaggcttccatggctgagaatagtatacctctgtacactaccgcttccatgggaaatcccactctgggcagcctggccagtgccatccgggaggagctgaacggggccatggagcacaccaacagcaacgagagtgacagcagtccaggcagatcccctatgcaagctgtgcaccccatacacgtcaaagaagaacccctcgaccccgaggaagctgaaggccctctgtccttagtgacaacagccaaccacagtccagattttgaccatgacagagattacgaagacgaaccagtaaatgaggacatggagtgaacctctgggcgggccgacccccgagaccgaagattggggaaaaaaaagcaaaacaaatttaaaaaaaaaataaaaaaagaaagaaaggaaaagaaaagaaaccacgtcaaaagttagcagtgaaacccgccctccgtttcttgtacagtctggaggattttcgctacattttgacaactctgaaacgtgttaactcttagtgccatcaagaatcccatttgggagtatttttgatttttctactttttgttgacaaaagggatttgtactctgtgcattggatggacctgtttggtacttgggattttcctctttgagtcaacatcagtgttgtaaattcgataaacggattcacttttagcagcagactttgaactgcagcttcgcccggctgatgctgggcgggcgccgaggacacccgactgagctcacgtacctgcgctgcagaccaagccttcgcccgagttcaagactccagtggacgacctttttgcacagcgctgcatgttgataccactgcctttactcactttggtttgttttgtttttgtttttcgcttccagttgggatgggggaaggcctttgtgtgtgtattggggggaggggttaaaaataattatcccaaattttttaatgtattgcttttttttttttcttttactgtttttttttctttctttctttttttttttggcttctttaccattttaagttctgacctcaggcctccatttgggcccagggcctcttggaggcttcgcgttttctgtaccttgtgatgaatgttaataggcgtttttattatacagagctgaatgtcatttctcgtctgtagttttctggcactcattccattttcctatagacatcaccatgtttctctcaagtttaaaacaaaacaaaacaaaacattccgttttggtctttttccaaaaaaaaaaaaagagaaaagaaaagaaaaaaaaagatcccaaaggctgcaccttacacctgaaggtccccacacggggacatgacatcctgccagtaagagaatgaatgacagaaaaggaaagagagaaacgcacgcgcgcacactctagggacatggcagattcatttcacaaaaacagtattgggggtgggatgggggactgttggaggtttttcttttttttctttttcatagcacccaccattgtgagtaactgccaccacattctctcagcatcaggaaacacagccacgaaagaaacagaagagaataaacaatcctcaatagttagactgtcacagtggccatggaagatcttccctcctataatcaggtgaccaggaccacaggccctgtgccatcctcacccgcacaggttgctctgtttgcaaaatgaccaaaaagccagcatggtgacagggctaagtttgtacagtagttcagacctgctcggtggcattcatctgttttaatgcaaatgagacttcacattgaacttgttatgggagttaatgaggactgagctcagcagacgctaatgtgtcaggttccagtgtgctaagtcggtactgcagcttggtctgcaatgtgagcaccacacccaccaccccagtagcacagacttcacggaagaggacaccaggtagacagaccagggcccacagaatgagatggctctaggtcaatatgccctcggcccttcaaacccctccacatttccgtctgcacccgcttccctggcatgtatttatctggggctgtagcttttgtttgggtttagtttgagagcctttcggagcttaatttatatactttgtacctttttttatttctaaaattaccaaagatattacgcaaaggtaaattattttcttttatactttatctgatgaagccaaatatcctcttattgttgatcaaaggaggcgaaaggattcagaggcaaatgatgaggtccaggctatttgccaacctgagggaaattactgcccgtcagcagagttcatttagcagactatgtaggcaatggaaccaaagttttttactttttttttcctgttttttttttttttctctttccttcttttctctacctgtagagagaccaaaactaactcctataaggagaaaatccggggctactgcagagaaaagaaatcaagacaacagtattatagctcctgtggaaagaatgggctagcatctcaaagaaaaagaaatgaatgtatgatgctgaaaattagtatataatcgaggagacacttaggatgaattcagagagaacggagaagtgtctggccgctcgaggccacttctagaataaagagatttttagaacattcgtacgccacttttgtttaagggctgtgctctgatcactgcgttaatcttggtttatcctggcatttatcctctggtttccttctgctttttgttgatgttttgttttatttttttcctatttcagattatgatttggtcagtgtttttcatttacacacaaaaaaagctaacattcccacccactcataagcttagagtagaacacctttggcaatgacttctgaaggtttgctctgctttctatagaggggcggtctgtggttattcatgccccctccaccaccaccccacccatcttttccaggcagcttcacagtgtgcaggcagtagccaggccgggtcatgggaagggggccctgtgcttctaaactgagtggttgctggttagatatacaaaagaggataaaactctggtaggttagttcattctcagcatgtgtagctagacaagagtaaagataacagcatggggactgttagtacgcacacctcagttcaaacctttagggaatgagtttaaaaaaaaaattaaaaatatacctttcactcagtcgtacttagtcgtatgtcttgcatgcttagtctaaagactgtagcaaagaaaataaataaaagagaaaaaattagattttatgtatcttttcaggtgtcatagcctggcagaagaaccaactgaaaaaaaaaaaaaaagttctcaggctttacagcaagcaatcttcactctgatttttagaattctgattctgtccctcggggggttcccgcttcgtttgaatggggttgattcggttgtacatatatcccgatgtgtctgtgtgcatctttgtcttttgcgggggagggccgaggggagggtttttttggtttcgttttgttttgttttgttttaattattttattttttaggtattgttcctaaaaaggaataaatgcatacacctgtttgtcaaaacatctttgctttttgtgcaactgcattttattaataatacatttaaaaaaagaagaggaagaaggaaaagaaaatagtatctttgagggaaaaaagtctactgtctgtaatggctttgcagttaatgctgcgtgtgtacttcattttattgcccttgatttacaaacattggatgacatgttgataatgggaagaaagaaaaactccacacacacatctctttctttccctccccctcctctctctctcaaattgtaacatagttgacaaaatgtgtttcctatcctcattatttggaacattttgtacaggtttgtggaatgggggtgagagagtgagtgtgtgagtgagtgtgtgttactgtttttgatacattcctagtccttgtgttttatcataccactgcctccttgactatcttaagttcctaagtttgaaaaaaggaaaaaaaaaaagttgtttaaaaaaaaaaatgttctctcttgctgcagtcaatatttttgcatgatgaaattccaggctcatactttcaaagttttatgagtaaagtagtgattaataatggggagtgttgaaatattgaactttttgtataaaaaaaatttttttacaaggtcccaaggtgtaaagacaatttgttacctttttctttctcttcttagtaaatattagcaaacgtgagggaggtcagcctgtgtcagacacacacagtcactgatgaggacccaccctccatgagcttaggggtctattcatgtgagaaggcttacagccatctcagggtgaacagcctaggcttacctgcatggtgacctagctgacaatcccctccccacatcaaaagacagttgagttcatattcctagcatctcatcagccataataagagaaagccttggtttaaaaaaataaaacaaaatgatagagccaaaagcattataaatcactagctctttttatttatttatttttttcagttacaatattgaagtctctcctctcttttccacattctagctgagctctctgtctccaagggcataaagcaaactctgctatttgaatcttgttggtacctgttggccagtgccataccctcctgacagtctggaagtgttttgcaacttggttttcctttattatcatgagaagcaaatggacaattctaacggcgtcttgtgggtccactgtaactcgaagcggtccctgctacccaggagcctgggtggagacggtgcggtctgggttgtgagtgtggtgctctttgtatctgtgctgccgctaacagggatcgttttgttttgttttgataagacataaaagaatctcattccttgatatcaactgtaattccaccattccatgtctgtggacacagacaataaaaaaaaatgttgtgtagtcagtagtaattactgacatgagcgcattcttaaatgcaataaaagtgctggtggttcacactggtagacaaagttaaaaaaaaaaaaaaaaa

SEQ ID NO: 2 - protein, 673 aa, Mus musculus
MMQESGSETKSNGSAIQNGSSGGNHLLECGALRDTRSNGEAPAVDLGAADLAHVQQQQQQQQQQQQQQQQQQQQQVSGLKSPKRNDKQPALQVPVSVAMMTPQVITPQQMQQILQQQVLSPQQLQVLLQQQQALMLQQQLQEFYKKQQEQLQLQLLQQQHAGKQPKEQQVATQQLAFQQQLLQMQQLQQQHLLSLQRQGLLTIQPGQPALPLQPLAQGMIPTELQQLWKEVTSAHTAEETTSSNHSSLDLTSTCVSSSAPSKSSLIMNPHASTNGQLSVHTPKRESLSHEEHPHSHPLYGHGVCKWPGCEAVCDDFPAFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGSLASAIREELNGAMEHTNSNESDSSPGRSPMQAVHPIHVKEEPLDPEEAEGPLSLVTTANHSPDFDHDRDYEDEPVNEDME

SEQ ID NO: 3 - RNA, 6649 nt, Mus musculus
  source 1..6649: mol_type = mRNA
ggcgcggcagcccgcgagcccgagcgccccttgcgcgtgcagtccgcctgcaccgaggccgccggacccagcccggccccgcgcgggcgccccgagccggctgcgcgcgcctggcggctacatggagagtgtcaggttcccgtgtcagtggctatgatgacacctcaggttatcactcctcaacaaatgcagcagatcctccagcagcaggtgctgagcccccagcagctccaggttctcctccagcagcagcaggccctcatgcttcaacagcagcttcaagaattttataaaaaacaacaggaacagttgcagcttcaacttctccaacagcaacatgctggaaaacagccgaaagagcagcagcaggtggctacccagcagttggctttccagcagcagcttctccagatgcagcagctgcagcagcagcacctcttgtctctccagcgccaaggcctcctaacaattcagccggggcagcctgcccttcccctccaacccctcgctcaaggcatgattccaacggaactgcagcagctctggaaagaagtgacaagtgcccacactgcagaggaaaccacaagcagcaaccacagcagcctagacctgaccagcacatgtgtctcgtcctcggcaccttccaagtcctccctaatcatgaacccgcatgcctctaccaatggacagctctcggtccacactcccaaaagggaaagcttgtcccacgaggagcacccccacagccaccctctctatggacatggcgtatgcaagtggccaggctgtgaggcggtttgtgacgacttcccagcctttctaaaacatctcaacagtgagcatgcgctggacgatagaagcacagctcaatgtagagtacaaatgcaggttgtacagcagttagagctacagcttgcaaaagacaaagagcgcctgcaagccatgatgacccacctgcatgtgaagtctacagaacccaaagctgcccctcagcccctgaatctggtatcaagtgtcaccctctccaagtctgcctcagaggcttctccacagagcttacctcatactccaacaacccccaccgcccccctgactcctgtcacccaaggcccctccgtcatcaccaccaccagcatgcacacggtgggacctatccgcaggcggtactcagacaaatacaacgtgcccatttcttcagcagatattgcgcagaaccaagaattttataagaacgcggaagttagaccaccatttacatatgcatctttaatcaggcaggccattctcgaatctccagaaaagcagctaacactaaacgaaatctataactggttcacacgaatgtttgcttacttccgacgcaatgcagccacgtggaagaatgcagtgcgtcataatcttagtctccacaagtgttttgtgcgagtagagaacgttaaaggggcagtatggacagtggatgaagtagagttccaaaaacggaggccacaaaagatcagtggtaacccttcccttattaaaaacatgcagagcagccacgcctactgcacacctctcaatgcagctttacaggcttccatggctgagaatagtatacctctgtacactaccgcttccatgggaaatcccactctgggcagcctggccagtgccatccgggaggagctgaacggggccatggagcacaccaacagcaacgagagtgacagcagtccaggcagatcccctatgcaagctgtgcaccccatacacgtcaaagaagaacccctcgaccccgaggaagctgaaggccctctgtccttagtgacaacagccaaccacagtccagattttgaccatgacagagattacgaagacgaaccagtaaatgaggacatggagtgaacctctgggcgggccgacccccgagaccgaagattggggaaaaaaaagcaaaacaaatttaaaaaaaaaataaaaaaagaaagaaaggaaaagaaaagaaaccacgtcaaaagttagcagtgaaacccgccctccgtttcttgtacagtctggaggattttcgctacattttgacaactctgaaacgtgttaactcttagtgccatcaagaatcccatttgggagtatttttgatttttctactttttgttgacaaaagggatttgtactctgtgcattggatggacctgtttggtacttgggattttcctctttgagtcaacatcagtgttgtaaattcgataaacggattcacttttagcagcagactttgaactgcagcttcgcccggctgatgctgggcgggcgccgaggacacccgactgagctcacgtacctgcgctgcagaccaagccttcgcccgagttcaagactccagtggacgacctttttgcacagcgctgcatgttgataccactgcctttactcactttggtttgttttgtttttgtttttcgcttccagttgggatgggggaaggcctttgtgtgtgtattggggggaggggttaaaaataattatcccaaattttttaatgtattgcttttttttttttcttttactgtttttttttctttctttctttttttttttggcttctttaccattttaagttctgacctcaggcctccatttgggcccagggcctcttggaggcttcgcgttttctgtaccttgtgatgaatgttaataggcgtttttattatacagagctgaatgtcatttctcgtctgtagttttctggcactcattccattttcctatagacatcaccatgtttctctcaagtttaaaacaaaacaaaacaaaacattccgttttggtctttttccaaaaaaaaaaaaagagaaaagaaaagaaaaaaaaagatcccaaaggctgcaccttacacctgaaggtccccacacggggacatgacatcctgccagtaagagaatgaatgacagaaaaggaaagagagaaacgcacgcgcgcacactctagggacatggcagattcatttcacaaaaacagtattgggggtgggatgggggactgttggaggtttttcttttttttctttttcatagcacccaccattgtgagtaactgccaccacattctctcagcatcaggaaacacagccacgaaagaaacagaagagaataaacaatcctcaatagttagactgtcacagtggccatggaagatcttccctcctataatcaggtgaccaggaccacaggccctgtgccatcctcacccgcacaggttgctctgtttgcaaaatgaccaaaaagccagcatggtgacagggctaagtttgtacagtagttcagacctgctcggtggcattcatctgttttaatgcaaatgagacttcacattgaacttgttatgggagttaatgaggactgagctcagcagacgctaatgtgtcaggttccagtgtgctaagtcggtactgcagcttggtctgcaatgtgagcaccacacccaccaccccagtagcacagacttcacggaagaggacaccaggtagacagaccagggcccacagaatgagatggctctaggtcaatatgccctcggcccttcaaacccctccacatttccgtctgcacccgcttccctggcatgtatttatctggggctgtagcttttgtttgggtttagtttgagagcctttcggagcttaatttatatactttgtacctttttttatttctaaaattaccaaagatattacgcaaaggtaaattattttcttttatactttatctgatgaagccaaatatcctcttattgttgatcaaaggaggcgaaaggattcagaggcaaatgatgaggtccaggctatttgccaacctgagggaaattactgcccgtcagcagagttcatttagcagactatgtaggcaatggaaccaaagttttttactttttttttcctgttttttttttttttctctttccttcttttctctacctgtagagagaccaaaactaactcctataaggagaaaatccggggctactgcagagaaaagaaatcaagacaacagtattatagctcctgtggaaagaatgggctagcatctcaaagaaaaagaaatgaatgtatgatgctgaaaattagtatataatcgaggagacacttaggatgaattcagagagaacggagaagtgtctggccgctcgaggccacttctagaataaagagatttttagaacattcgtacgccacttttgtttaagggctgtgctctgatcactgcgttaatcttggtttatcctggcatttatcctctggtttccttctgctttttgttgatgttttgttttatttttttcctatttcagattatgatttggtcagtgtttttcatttacacacaaaaaaagctaacattcccacccactcataagcttagagtagaacacctttggcaatgacttctgaaggtttgctctgctttctatagaggggcggtctgtggttattcatgccccctccaccaccaccccacccatcttttccaggcagcttcacagtgtgcaggcagtagccaggccgggtcatgggaagggggccctgtgcttctaaactgagtggttgctggttagatatacaaaagaggataaaactctggtaggttagttcattctcagcatgtgtagctagacaagagtaaagataacagcatggggactgttagtacgcacacctcagttcaaacctttagggaatgagtttaaaaaaaaaattaaaaatatacctttcactcagtcgtacttagtcgtatgtcttgcatgcttagtctaaagactgtagcaaagaaaataaataaaagagaaaaaattagattttatgtatcttttcaggtgtcatagcctggcagaagaaccaactgaaaaaaaaaaaaaaagttctcaggctttacagcaagcaatcttcactctgatttttagaattctgattctgtccctcggggggttcccgcttcgtttgaatggggttgattcggttgtacatatatcccgatgtgtctgtgtgcatctttgtcttttgcgggggagggccgaggggagggtttttttggtttcgttttgttttgttttgttttaattattttattttttaggtattgttcctaaaaaggaataaatgcatacacctgtttgtcaaaacatctttgctttttgtgcaactgcattttattaataatacatttaaaaaaagaagaggaagaaggaaaagaaaatagtatctttgagggaaaaaagtctactgtctgtaatggctttgcagttaatgctgcgtgtgtacttcattttattgcccttgatttacaaacattggatgacatgttgataatgggaagaaagaaaaactccacacacacatctctttctttccctccccctcctctctctctcaaattgtaacatagttgacaaaatgtgtttcctatcctcattatttggaacattttgtacaggtttgtggaatgggggtgagagagtgagtgtgtgagtgagtgtgtgttactgtttttgatacattcctagtccttgtgttttatcataccactgcctccttgactatcttaagttcctaagtttgaaaaaaggaaaaaaaaaaagttgtttaaaaaaaaaaatgttctctcttgctgcagtcaatatttttgcatgatgaaattccaggctcatactttcaaagttttatgagtaaagtagtgattaataatggggagtgttgaaatattgaactttttgtataaaaaaaatttttttacaaggtcccaaggtgtaaagacaatttgttacctttttctttctcttcttagtaaatattagcaaacgtgagggaggtcagcctgtgtcagacacacacagtcactgatgaggacccaccctccatgagcttaggggtctattcatgtgagaaggcttacagccatctcagggtgaacagcctaggcttacctgcatggtgacctagctgacaatcccctccccacatcaaaagacagttgagttcatattcctagcatctcatcagccataataagagaaagccttggtttaaaaaaataaaacaaaatgatagagccaaaagcattataaatcactagctctttttatttatttatttttttcagttacaatattgaagtctctcctctcttttccacattctagctgagctctctgtctccaagggcataaagcaaactctgctatttgaatcttgttggtacctgttggccagtgccataccctcctgacagtctggaagtgttttgcaacttggttttcctttattatcatgagaagcaaatggacaattctaacggcgtcttgtgggtccactgtaactcgaagcggtccctgctacccaggagcctgggtggagacggtgcggtctgggttgtgagtgtggtgctctttgtatctgtgctgccgctaacagggatcgttttgttttgttttgataagacataaaagaatctcattccttgatatcaactgtaattccaccattccatgtctgtggacacagacaataaaaaaaaatgttgtgtagtcagtagtaattactgacatgagcgcattcttaaatgcaataaaagtgctggtggttcacactggtagacaaagttaaaaaaaaaaaaaaaaa

SEQ ID NO: 4 - protein, 576 aa, Mus musculus
MMTPQVITPQQMQQILQQQVLSPQQLQVLLQQQQALMLQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTSSNHSSLDLTSTCVSSSAPSKSSLIMNPHASTNGQLSVHTPKRESLSHEEHPHSHPLYGHGVCKWPGCEAVCDDFPAFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGSLASAIREELNGAMEHTNSNESDSSPGRSPMQAVHPIHVKEEPLDPEEAEGPLSLVTTANHSPDFDHDRDYEDEPVNEDME

SEQ ID NO: 5 - RNA, 7570 nt, Mus musculus
  source 1..7570: mol_type = mRNA
actttgtaagatcccgggaaggtgctgagactaaggctcaggccgggctccgggtttcccgatccctgcagggatcccccgcggcgcatcctctttccctattccctggagctcgggggtcctctccttcttgctgcaccttctatctttgctcctggccccctccgcctaatcacctcgtccccttcgagccccccccccccacaaccttcacggaaggaagaattgctgagggtcgccccggtctcttgtcgatcgcagaccccgaaactttgccccaggaggacggcgcgcggcgggcgagcgggcgagcgggcgaggggcagcggcggcggcggcggcggcggcggcgcggagaagcgtggggctgccggcgcctcctccccggactcgcgtcccaggagcttctgctgactctcctggtttccgagggaaccctttcggccatggtgagggctgcgctcccttctctgccttgcggtcaaccatgagcggatgggtgtgatgcctgctagcctaaagggctccttctgtgtgctgcagtcttgtggcgttctgcaggccccctgccagtgaccccaggctttttatggctgtgagactcattaaaagctcaggggtgagacgtgacctttggaggtgactataactgaagattgaattacagaagccaaaaaattaaataaataaaaggtttttgagtcatgatgcaagaatctgggtctgagacaaaaagtaacggatcagccatccagaacgggtccagcggtggcaaccacttactagagtgcggggcacttcgtgacactcggtccaacggagaggcaccagcggtggacctgggggcagccgaccttgcccacgtccagcagcagcaacagcaggccctgcaggtggcaaggcagctcctccttcagcagcagcaacagcagcagcagcaacagcagcagcagcagcagcagcaacagcagcaacagcagcagcaacaacagcagcagcagcaacagcagcagcagcagcagcaagttagtggattaaagtctcccaagaggaatgacaagcaaccagctcttcaggttcccgtgtcagtggctatgatgacacctcaggttatcactcctcaacaaatgcagcagatcctccagcagcaggtgctgagcccccagcagctccaggttctcctccagcagcagcaggccctcatgcttcaacagcagcttcaagaattttataaaaaacaacaggaacagttgcagcttcaacttctccaacagcaacatgctggaaaacagccgaaagagcagcagcaggtggctacccagcagttggctttccagcagcagcttctccagatgcagcagctgcagcagcagcacctcttgtctctccagcgccaaggcctcctaacaattcagccggggcagcctgcccttcccctccaacccctcgctcaaggcatgattccaacggaactgcagcagctctggaaagaagtgacaagtgcccacactgcagaggaaaccacaagcagcaaccacagcagcctagacctgaccagcacatgtgtctcgtcctcggcaccttccaagtcctccctaatcatgaacccgcatgcctctaccaatggacagctctcggtccacactcccaaaagggaaagcttgtcccacgaggagcacccccacagccaccctctctatggacatggcgtatgcaagtggccaggctgtgaggcggtttgtgacgacttcccagcctttctaaaacatctcaacagtgagcatgcgctggacgatagaagcacagctcaatgtagagtacaaatgcaggttgtacagcagttagagctacagcttgcaaaagacaaagagcgcctgcaagccatgatgacccacctgcatgtgaagtctacagaacccaaagctgcccctcagcccctgaatctggtatcaagtgtcaccctctccaagtctgcctcagaggcttctccacagagcttacctcatactccaacaacccccaccgcccccctgactcctgtcacccaaggcccctccgtcatcaccaccaccagcatgcacacggtgggacctatccgcaggcggtactcagacaaatacaacgtgcccatttcttcagatattgcgcagaaccaagaattttataagaacgcggaagttagaccaccatttacatatgcatctttaatcaggcaggccattctcgaatctccagaaaagcagctaacactaaacgaaatctataactggttcacacgaatgtttgcttacttccgacgcaatgcagccacgtggaagaatgcagtgcgtcataatcttagtctccacaagtgttttgtgcgagtagagaacgttaaaggggcagtatggacagtggatgaagtagagttccaaaaacggaggccacaaaagatcagtggtaacccttcccttattaaaaacatgcagagcagccacgcctactgcacacctctcaatgcagctttacaggcttccatggctgagaatagtatacctctgtacactaccgcttccatgggaaatcccactctgggcagcctggccagtgccatccgggaggagctgaacggggccatggagcacaccaacagcaacgagagtgacagcagtccaggcagatcccctatgcaagctgtgcaccccatacacgtcaaagaagaacccctcgaccccgaggaagctgaaggccctctgtccttagtgacaacagccaaccacagtccagattttgaccatgacagagattacgaagacgaaccagtaaatgaggacatggagtgaacctctgggcgggccgacccccgagaccgaagattggggaaaaaaaagcaaaacaaatttaaaaaaaaaataaaaaaagaaagaaaggaaaagaaaagaaaccacgtcaaaagttagcagtgaaacccgccctccgtttcttgtacagtctggaggattttcgctacattttgacaactctgaaacgtgttaactcttagtgccatcaagaatcccatttgggagtatttttgatttttctactttttgttgacaaaagggatttgtactctgtgcattggatggacctgtttggtacttgggattttcctctttgagtcaacatcagtgttgtaaattcgataaacggattcacttttagcagcagactttgaactgcagcttcgcccggctgatgctgggcgggcgccgaggacacccgactgagctcacgtacctgcgctgcagaccaagccttcgcccgagttcaagactccagtggacgacctttttgcacagcgctgcatgttgataccactgcctttactcactttggtttgttttgtttttgtttttcgcttccagttgggatgggggaaggcctttgtgtgtgtattggggggaggggttaaaaataattatcccaaattttttaatgtattgcttttttttttttcttttactgtttttttttctttctttctttttttttttggcttctttaccattttaagttctgacctcaggcctccatttgggcccagggcctcttggaggcttcgcgttttctgtaccttgtgatgaatgttaataggcgtttttattatacagagctgaatgtcatttctcgtctgtagttttctggcactcattccattttcctatagacatcaccatgtttctctcaagtttaaaacaaaacaaaacaaaacattccgttttggtctttttccaaaaaaaaaaaaagagaaaagaaaagaaaaaaaaagatcccaaaggctgcaccttacacctgaaggtccccacacggggacatgacatcctgccagtaagagaatgaatgacagaaaaggaaagagagaaacgcacgcgcgcacactctagggacatggcagattcatttcacaaaaacagtattgggggtgggatgggggactgttggaggtttttcttttttttctttttcatagcacccaccattgtgagtaactgccaccacattctctcagcatcaggaaacacagccacgaaagaaacagaagagaataaacaatcctcaatagttagactgtcacagtggccatggaagatcttccctcctataatcaggtgaccaggaccacaggccctgtgccatcctcacccgcacaggttgctctgtttgcaaaatgaccaaaaagccagcatggtgacagggctaagtttgtacagtagttcagacctgctcggtggcattcatctgttttaatgcaaatgagacttcacattgaacttgttatgggagttaatgaggactgagctcagcagacgctaatgtgtcaggttccagtgtgctaagtcggtactgcagcttggtctgcaatgtgagcaccacacccaccaccccagtagcacagacttcacggaagaggacaccaggtagacagaccagggcccacagaatgagatggctctaggtcaatatgccctcggcccttcaaacccctccacatttccgtctgcacccgcttccctggcatgtatttatctggggctgtagcttttgtttgggtttagtttgagagcctttcggagcttaatttatatactttgtacctttttttatttctaaaattaccaaagatattacgcaaaggtaaattattttcttttatactttatctgatgaagccaaatatcctcttattgttgatcaaaggaggcgaaaggattcagaggcaaatgatgaggtccaggctatttgccaacctgagggaaattactgcccgtcagcagagttcatttagcagactatgtaggcaatggaaccaaagttttttactttttttttcctgttttttttttttttctctttccttcttttctctacctgtagagagaccaaaactaactcctataaggagaaaatccggggctactgcagagaaaagaaatcaagacaacagtattatagctcctgtggaaagaatgggctagcatctcaaagaaaaagaaatgaatgtatgatgctgaaaattagtatataatcgaggagacacttaggatgaattcagagagaacggagaagtgtctggccgctcgaggccacttctagaataaagagatttttagaacattcgtacgccacttttgtttaagggctgtgctctgatcactgcgttaatcttggtttatcctggcatttatcctctggtttccttctgctttttgttgatgttttgttttatttttttcctatttcagattatgatttggtcagtgtttttcatttacacacaaaaaaagctaacattcccacccactcataagcttagagtagaacacctttggcaatgacttctgaaggtttgctctgctttctatagaggggcggtctgtggttattcatgccccctccaccaccaccccacccatcttttccaggcagcttcacagtgtgcaggcagtagccaggccgggtcatgggaagggggccctgtgcttctaaactgagtggttgctggttagatatacaaaagaggataaaactctggtaggttagttcattctcagcatgtgtagctagacaagagtaaagataacagcatggggactgttagtacgcacacctcagttcaaacctttagggaatgagtttaaaaaaaaaattaaaaatatacctttcactcagtcgtacttagtcgtatgtcttgcatgcttagtctaaagactgtagcaaagaaaataaataaaagagaaaaaattagattttatgtatcttttcaggtgtcatagcctggcagaagaaccaactgaaaaaaaaaaaaaaagttctcaggctttacagcaagcaatcttcactctgatttttagaattctgattctgtccctcggggggttcccgcttcgtttgaatggggttgattcggttgtacatatatcccgatgtgtctgtgtgcatctttgtcttttgcgggggagggccgaggggagggtttttttggtttcgttttgttttgttttgttttaattattttattttttaggtattgttcctaaaaaggaataaatgcatacacctgtttgtcaaaacatctttgctttttgtgcaactgcattttattaataatacatttaaaaaaagaagaggaagaaggaaaagaaaatagtatctttgagggaaaaaagtctactgtctgtaatggctttgcagttaatgctgcgtgtgtacttcattttattgcccttgatttacaaacattggatgacatgttgataatgggaagaaagaaaaactccacacacacatctctttctttccctccccctcctctctctctcaaattgtaacatagttgacaaaatgtgtttcctatcctcattatttggaacattttgtacaggtttgtggaatgggggtgagagagtgagtgtgtgagtgagtgtgtgttactgtttttgatacattcctagtccttgtgttttatcataccactgcctccttgactatcttaagttcctaagtttgaaaaaaggaaaaaaaaaaagttgtttaaaaaaaaaaatgttctctcttgctgcagtcaatatttttgcatgatgaaattccaggctcatactttcaaagttttatgagtaaagtagtgattaataatggggagtgttgaaatattgaactttttgtataaaaaaaatttttttacaaggtcccaaggtgtaaagacaatttgttacctttttctttctcttcttagtaaatattagcaaacgtgagggaggtcagcctgtgtcagacacacacagtcactgatgaggacccaccctccatgagcttaggggtctattcatgtgagaaggcttacagccatctcagggtgaacagcctaggcttacctgcatggtgacctagctgacaatcccctccccacatcaaaagacagttgagttcatattcctagcatctcatcagccataataagagaaagccttggtttaaaaaaataaaacaaaatgatagagccaaaagcattataaatcactagctctttttatttatttatttttttcagttacaatattgaagtctctcctctcttttccacattctagctgagctctctgtctccaagggcataaagcaaactctgctatttgaatcttgttggtacctgttggccagtgccataccctcctgacagtctggaagtgttttgcaacttggttttcctttattatcatgagaagcaaatggacaattctaacggcgtcttgtgggtccactgtaactcgaagcggtccctgctacccaggagcctgggtggagacggtgcggtctgggttgtgagtgtggtgctctttgtatctgtgctgccgctaacagggatcgttttgttttgttttgataagacataaaagaatctcattccttgatatcaactgtaattccaccattccatgtctgtggacacagacaataaaaaaaaatgttgtgtagtcagtagtaattactgacatgagcgcattcttaaatgcaataaaagtgctggtggttcacactggtagacaaagtt

SEQ ID NO: 6 - protein, 705 aa, Mus musculus
MMQESGSETKSNGSAIQNGSSGGNHLLECGALRDTRSNGEAPAVDLGAADLAHVQQQQQQALQVARQLLLQQQQQQQQQQQQQQQQQQQQQQQQQQQQQQQQQQQQQVSGLKSPKRNDKQPALQVPVSVAMMTPQVITPQQMQQILQQQVLSPQQLQVLLQQQQALMLQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTSSNHSSLDLTSTCVSSSAPSKSSLIMNPHASTNGQLSVHTPKRESLSHEEHPHSHPLYGHGVCKWPGCEAVCDDFPAFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSDIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGSLASAIREELNGAMEHTNSNESDSSPGRSPMQAVHPIHVKEEPLDPEEAEGPLSLVTTANHSPDFDHDRDYEDEPVNEDME

SEQ ID NO: 7 - RNA, 7177 nt, Mus musculus
  source 1..7177: mol_type = mRNA
cgtcccaggagcttctgctgactctcctggtttccgagggaaccctttcggccatggtgagggctgcgctcccttctctgccttgcggtcaaccatgagcggatgggtgtgatgcctgctagcctaaagggctccttctgtgtgctgcagtcttgtggcgttctgcaggccccctgccagtgaccccaggctttttatggctgtgagactcattaaaagctcaggggtgagacgtgacctttggaggtgactataactgaagattgaattacagaagccaaaaaattaaataaataaaaggtttttgagtcatgatgcaagaatctgggtctgagacaaaaagtaacggatcagccatccagaacgggtccagcggtggcaaccacttactagagtgcggggcacttcgtgacactcggtccaacggagaggcaccagcggtggacctgggggcagccgaccttgcccacgtccagcagcagcaacagcaggccctgcaggtggcaaggcagctcctccttcagcagcagcaacagcagcagcagcaacagcagcagcagcagcagcagcaacagcagcaacagcagcagcaacaacagcagcagcagcaacagcagcagcagcagcagcaagttagtggattaaagtctcccaagaggaatgacaagcaaccagctcttcaggttcccgtgtcagtggctatgatgacacctcaggttatcactcctcaacaaatgcagcagatcctccagcagcaggtgctgagcccccagcagctccaggttctcctccagcagcagcaggccctcatgcttcaacagcagcttcaagaattttataaaaaacaacaggaacagttgcagcttcaacttctccaacagcaacatgctggaaaacagccgaaagagcagcaggtggctacccagcagttggctttccagcagcagcttctccagatgcagcagctgcagcagcagcacctcttgtctctccagcgccaaggcctcctaacaattcagccggggcagcctgcccttcccctccaacccctcgctcaaggcatgattccaacggaactgcagcagctctggaaagaagtgacaagtgcccacactgcagaggaaaccacaagcagcaaccacagcagcctagacctgaccagcacatgtgtctcgtcctcggcaccttccaagtcctccctaatcatgaacccgcatgcctctaccaatggacagctctcggtccacactcccaaaagggaaagcttgtcccacgaggagcacccccacagccaccctctctatggacatggcgtatgcaagtggccaggctgtgaggcggtttgtgacgacttcccagcctttctaaaacatctcaacagtgagcatgcgctggacgatagaagcacagctcaatgtagagtacaaatgcaggttgtacagcagttagagctacagcttgcaaaagacaaagagcgcctgcaagccatgatgacccacctgcatgtgaagtctacagaacccaaagctgcccctcagcccctgaatctggtatcaagtgtcaccctctccaagtctgcctcagaggcttctccacagagcttacctcatactccaacaacccccaccgcccccctgactcctgtcacccaaggcccctccgtcatcaccaccaccagcatgcacacggtgggacctatccgcaggcggtactcagacaaatacaacgtgcccatttcttcagcagatattgcgcagaaccaagaattttataagaacgcggaagttagaccaccatttacatatgcatctttaatcaggcaggccattctcgaatctccagaaaagcagctaacactaaacgaaatctataactggttcacacgaatgtttgcttacttccgacgcaatgcagccacgtggaagaatgcagtgcgtcataatcttagtctccacaagtgttttgtgcgagtagagaacgttaaaggggcagtatggacagtggatgaagtagagttccaaaaacggaggccacaaaagatcagtggtaacccttcccttattaaaaacatgcagagcagccacgcctactgcacacctctcaatgcagctttacaggcttccatggctgagaatagtatacctctgtacactaccgcttccatgggaaatcccactctgggcagcctggccagtgccatccgggaggagctgaacggggccatggagcacaccaacagcaacgagagtgacagcagtccaggcagatcccctatgcaagctgtgcaccccatacacgtcaaagaagaacccctcgaccccgaggaagctgaaggccctctgtccttagtgacaacagccaaccacagtccagattttgaccatgacagagattacgaagacgaaccagtaaatgaggacatggagtgaacctctgggcgggccgacccccgagaccgaagattggggaaaaaaaagcaaaacaaatttaaaaaaaaaataaaaaaagaaagaaaggaaaagaaaagaaaccacgtcaaaagttagcagtgaaacccgccctccgtttcttgtacagtctggaggattttcgctacattttgacaactctgaaacgtgttaactcttagtgccatcaagaatcccatttgggagtatttttgatttttctactttttgttgacaaaagggatttgtactctgtgcattggatggacctgtttggtacttgggattttcctctttgagtcaacatcagtgttgtaaattcgataaacggattcacttttagcagcagactttgaactgcagcttcgcccggctgatgctgggcgggcgccgaggacacccgactgagctcacgtacctgcgctgcagaccaagccttcgcccgagttcaagactccagtggacgacctttttgcacagcgctgcatgttgataccactgcctttactcactttggtttgttttgtttttgtttttcgcttccagttgggatgggggaaggcctttgtgtgtgtattggggggaggggttaaaaataattatcccaaattttttaatgtattgcttttttttttttcttttactgtttttttttctttctttctttttttttttggcttctttaccattttaagttctgacctcaggcctccatttgggcccagggcctcttggaggcttcgcgttttctgtaccttgtgatgaatgttaataggcgtttttattatacagagctgaatgtcatttctcgtctgtagttttctggcactcattccattttcctatagacatcaccatgtttctctcaagtttaaaacaaaacaaaacaaaacattccgttttggtctttttccaaaaaaaaaaaaagagaaaagaaaagaaaaaaaaagatcccaaaggctgcaccttacacctgaaggtccccacacggggacatgacatcctgccagtaagagaatgaatgacagaaaaggaaagagagaaacgcacgcgcgcacactctagggacatggcagattcatttcacaaaaacagtattgggggtgggatgggggactgttggaggtttttcttttttttctttttcatagcacccaccattgtgagtaactgccaccacattctctcagcatcaggaaacacagccacgaaagaaacagaagagaataaacaatcctcaatagttagactgtcacagtggccatggaagatcttccctcctataatcaggtgaccaggaccacaggccctgtgccatcctcacccgcacaggttgctctgtttgcaaaatgaccaaaaagccagcatggtgacagggctaagtttgtacagtagttcagacctgctcggtggcattcatctgttttaatgcaaatgagacttcacattgaacttgttatgggagttaatgaggactgagctcagcagacgctaatgtgtcaggttccagtgtgctaagtcggtactgcagcttggtctgcaatgtgagcaccacacccaccaccccagtagcacagacttcacggaagaggacaccaggtagacagaccagggcccacagaatgagatggctctaggtcaatatgccctcggcccttcaaacccctccacatttccgtctgcacccgcttccctggcatgtatttatctggggctgtagcttttgtttgggtttagtttgagagcctttcggagcttaatttatatactttgtacctttttttatttctaaaattaccaaagatattacgcaaaggtaaattattttcttttatactttatctgatgaagccaaatatcctcttattgttgatcaaaggaggcgaaaggattcagaggcaaatgatgaggtccaggctatttgccaacctgagggaaattactgcccgtcagcagagttcatttagcagactatgtaggcaatggaaccaaagttttttactttttttttcctgttttttttttttttctctttccttcttttctctacctgtagagagaccaaaactaactcctataaggagaaaatccggggctactgcagagaaaagaaatcaagacaacagtattatagctcctgtggaaagaatgggctagcatctcaaagaaaaagaaatgaatgtatgatgctgaaaattagtatataatcgaggagacacttaggatgaattcagagagaacggagaagtgtctggccgctcgaggccacttctagaataaagagatttttagaacattcgtacgccacttttgtttaagggctgtgctctgatcactgcgttaatcttggtttatcctggcatttatcctctggtttccttctgctttttgttgatgttttgttttatttttttcctatttcagattatgatttggtcagtgtttttcatttacacacaaaaaaagctaacattcccacccactcataagcttagagtagaacacctttggcaatgacttctgaaggtttgctctgctttctatagaggggcggtctgtggttattcatgccccctccaccaccaccccacccatcttttccaggcagcttcacagtgtgcaggcagtagccaggccgggtcatgggaagggggccctgtgcttctaaactgagtggttgctggttagatatacaaaagaggataaaactctggtaggttagttcattctcagcatgtgtagctagacaagagtaaagataacagcatggggactgttagtacgcacacctcagttcaaacctttagggaatgagtttaaaaaaaaaattaaaaatatacctttcactcagtcgtacttagtcgtatgtcttgcatgcttagtctaaagactgtagcaaagaaaataaataaaagagaaaaaattagattttatgtatcttttcaggtgtcatagcctggcagaagaaccaactgaaaaaaaaaaaaaaagttctcaggctttacagcaagcaatcttcactctgatttttagaattctgattctgtccctcggggggttcccgcttcgtttgaatggggttgattcggttgtacatatatcccgatgtgtctgtgtgcatctttgtcttttgcgggggagggccgaggggagggtttttttggtttcgttttgttttgttttgttttaattattttattttttaggtattgttcctaaaaaggaataaatgcatacacctgtttgtcaaaacatctttgctttttgtgcaactgcattttattaataatacatttaaaaaaagaagaggaagaaggaaaagaaaatagtatctttgagggaaaaaagtctactgtctgtaatggctttgcagttaatgctgcgtgtgtacttcattttattgcccttgatttacaaacattggatgacatgttgataatgggaagaaagaaaaactccacacacacatctctttctttccctccccctcctctctctctcaaattgtaacatagttgacaaaatgtgtttcctatcctcattatttggaacattttgtacaggtttgtggaatgggggtgagagagtgagtgtgtgagtgagtgtgtgttactgtttttgatacattcctagtccttgtgttttatcataccactgcctccttgactatcttaagttcctaagtttgaaaaaaggaaaaaaaaaaagttgtttaaaaaaaaaaatgttctctcttgctgcagtcaatatttttgcatgatgaaattccaggctcatactttcaaagttttatgagtaaagtagtgattaataatggggagtgttgaaatattgaactttttgtataaaaaaaatttttttacaaggtcccaaggtgtaaagacaatttgttacctttttctttctcttcttagtaaatattagcaaacgtgagggaggtcagcctgtgtcagacacacacagtcactgatgaggacccaccctccatgagcttaggggtctattcatgtgagaaggcttacagccatctcagggtgaacagcctaggcttacctgcatggtgacctagctgacaatcccctccccacatcaaaagacagttgagttcatattcctagcatctcatcagccataataagagaaagccttggtttaaaaaaataaaacaaaatgatagagccaaaagcattataaatcactagctctttttatttatttatttttttcagttacaatattgaagtctctcctctcttttccacattctagctgagctctctgtctccaagggcataaagcaaactctgctatttgaatcttgttggtacctgttggccagtgccataccctcctgacagtctggaagtgttttgcaacttggttttcctttattatcatgagaagcaaatggacaattctaacggcgtcttgtgggtccactgtaactcgaagcggtccctgctacccaggagcctgggtggagacggtgcggtctgggttgtgagtgtggtgctctttgtatctgtgctgccgctaacagggatcgttttgttttgttttgataagacataaaagaatctcattccttgatatcaactgtaattccaccattccatgtctgtggacacagacaataaaaaaaaatgttgtgtagtcagtagtaattactgacatgagcgcattcttaaatgcaataaaagtgctggtggttcacactggtagacaaagtt

SEQ ID NO: 8 - protein, 705 aa, Mus musculus
MMQESGSETKSNGSAIQNGSSGGNHLLECGALRDTRSNGEAPAVDLGAADLAHVQQQQQQALQVARQLLLQQQQQQQQQQQQQQQQQQQQQQQQQQQQQQQQQQQQQVSGLKSPKRNDKQPALQVPVSVAMMTPQVITPQQMQQILQQQVLSPQQLQVLLQQQQALMLQQQLQEFYKKQQEQLQLQLLQQQHAGKQPKEQQVATQQLAFQQQLLQMQQLQQQHLLSLQRQGLLTIQPGQPALPLQPLAQGMIPTELQQLWKEVTSAHTAEETTSSNHSSLDLTSTCVSSSAPSKSSLIMNPHASTNGQLSVHTPKRESLSHEEHPHSHPLYGHGVCKWPGCEAVCDDFPAFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGSLASAIREELNGAMEHTNSNESDSSPGRSPMQAVHPIHVKEEPLDPEEAEGPLSLVTTANHSPDFDHDRDYEDEPVNEDME

SEQ ID NO: 9 - RNA, 6760 nt, Mus musculus
  source 1..6760: mol_type = mRNA
cacacacacacacatacaagcgcgcgcgcacacacatacacacagacacacacacacaccaacacacacatacatacagagatccgggttgtggggggagctgccagcgcgagacagcgcgagcctccgagaaagcgcgagacacgccggcgcgtgcagctccccggccgcagcttcgtcccggccggagcccggcgccaccagcagttcatccgtcagtgcccgcccagggcatttatgccgcggccgcgacttcccgccgcagcttcctcgggtcccccctcccctggtgtccccgaactcgcggccgcggctgccgggactcgagcgtgggtgtgttgttccggggcttctgcctgggtgcacagcgggtgccacctcctgggacgctgccctcggggtccccgatcgcgaccaaacaagtaagagaactctgttgaagctcgatcctcacctactctctaaataaaagaatattccagagaaggaaagagagaagcacaaatattaagacttctagacccctggagaggactaaagtctacccctccacatggcttcttctccaatgtgatgctggagttgaatgaacttaggactccactccaaggttgacttgaaaggtgtgtagaagattggaaagagaaaggtattaagtcatgatgcaggaatctgcgacagagacaataagcaacagttcaatgaatcaaaatggaatgagcactttaagcagccaattagatgctggcagcagggatgggagatcaagtggtgacaccagctctgaagtaagcacagtggagctgctgcatctgcaacaacagcaggctctccaggcagcaagacaacttttgttacagcagcaaaccagtggattgaaatctcctaagagcagtgagaagcagaggccactgcaggtgcccgtgtcggtggccatgatgactccccaagtgatcacccctcagcagatgcagcagattcttcagcagcaggtcctgtctcctcagcagctccaggccctcctccagcagcagcaagctgtgatgttgcagcagcaacaactacaagagttttacaagaaacagcaagaacagttacatcttcagcttttgcaacagcagcaacagcagcagcagcaacagcagcaacagcagcagcagcagcagcaacaacagcagcagcagcagcaacagcaacagcagcagcagcaacagcagcagcaacagcaacagcatcctggaaagcaagcaaaagagcagcagcagcaacagcagcagcagcagttggcggcgcagcagctcgtcttccagcagcagcttctccaaatgcagcagctacagcagcaacaacatctgctcagccttcagcgccagggcctcatctccatcccacccggccaagcagcccttcctgtccagtcgctgcctcaagctggcttaagtcctgctgagattcagcaactatggaaagaagtgactggagtccatagtatggaagacaacggcatcaagcatggagggctagacctcacgactaacaattcctcctcgactacctcctccaccacgtccaaagcatcaccacccatcacacatcattccatagtgaacggacagtcttcagttctgaatgcaaggcgggacagctcatcacatgaggagactggggcctcgcacactctctatggccatggagtctgcaagtggccaggctgtgaaagcatatgtgaagattttggacagtttttgaagcaccttaacaatgagcatgcattggatgaccgaagcactgcccaatgtcgagtgcaaatgcaggtggtacagcagttagaaatacagctttctaaggaacgcgaacgtcttcaagcgatgatgacccacttgcacatgcgaccctcagagcccaaaccatctcccaaacctctaaatctggtgtctagtgtcaccatgtcgaagaacatgctggagacatccccacagagcttacctcaaacccctaccacaccaacggccccagtcaccccgattacccagggaccctctgtaatcaccccagccagtgtgcccaatgtgggagccatacgaaggcgacattcagacaaatacaacatccccatgtcatcagagattgccccaaactacgaattttataaaaacgcagatgttagacctcccttcacttatgcaacactcataaggcaggctatcatggagtcatctgacaggcagttaacacttaatgaaatttacagctggtttacacggacatttgcttacttcaggcggaatgccgccacttggaagaatgcagtgcgtcataatcttagcctgcacaaatgttttgttcgagtagaaaatgttaaaggagcagtgtggactgtggacgaagtcgaataccagaagcgaaggtcacaaaagataacaggaagtccaactttagtaaaaaatatacctacaagtttgggctatggagcagctctaaatgccagtctgcaggctgccttggcagaaagcagcttgcctttgctaagtaaccctggactgatcaataacgcctccagtggcctcctgcaggccgtccacgaagacctcaatggttccctggaccacatcgacagcaatgggaacagcagtccgggttgctcacctcagccacacatacattcaatccatgtcaaggaagaacccgtgattgcagaggatgaagactgtccaatgtcattagtgacaacagccaatcacagtccggagttagaagatgacagagagattgaggaagagcctttatctgaggacctggaatgagaacgaacttgtgacacctcagtgtgaagggacatatcactgaccttcataaccactccacaaccatgaatagttgacaaatttttactgtgactatttattaagcatggataaaggagacagccctaaaggaacttactaagccagccctttgggattcagaaccaacaggcaaattgcttgttttcttctctttctgtccttattttgtttgtttgtttgtttgattttactttctttacttattttctttttttcatttttttaaaggcaaaaaaaaaaactgattttcttgggaaaagagagaaagaactgttctttctataatggctttgcccatttaaaaaatgtggctctcaagggttcatgaaatgactgagtatgaggatccatgtcctatagaaagcaactgcgcctcatatactgccaaaaatagtgttagtttcattaatgtgaattttccagcattcagtagtcgtaatgttagaaacaattgctggtcaagttcaacttgttgctattgtttttaatttgcacaggagtagtatcagaaattagtgtcactgcttgtatctagctgacttttaaacaacagaacattagttttttatgttggtgccaccaactgtaaatgacataagttagttattacaaaacacagtaattagactgttgcaaccatctaaaaccttaggcttccagtctgtgctgttagtgtaaagatgtaaagtgcaatcctaagctaacattatctgtgcaagcaccatagaaacatttgcatatctgcatagatcttacaactgtactctttacctccttgtgataaagctttgtctacctgcaaacacagtcaaaggctacagctgcaaaccaaagccaactctaaccatggccaagagctcaaggacagaagcagccacatgctttggtcagccttctgtaacttcaattagtacaaaggaaccttttccatgaactacctgctgttttctgatgacctctgggatcttttcatttagccctaaacaaagaaacgaatatgacaaaacaaaaacaaaaaaacaacccacaactaagagttcattcagtcacagagtaatcttctgaggccaaaagtccatctaaatgcaatgaagatttgctttcattaaagacagaggtgaggacaaaatctgcagtggaagttatgacactctagaaagcaacaaatgtggatcactgaccaaaacaattatgtacttgatgcaaatgcagattgcgtattgttatatatatagtacattgcgtttttgttttccccatctttttggtggtgagttcatgttgttagaagtacatgtcttgtatggtcttaatctctgttgtgtactatttttttatagtcttaagttataataaaaaaaaacaaaaaacaaaaaacaaaaagtaggaaccaaacataaaaggtctagtaaagccaaaaattaatttcctattgattttaaagtgatctagctgagtttttacactgaaagcaaagattatagcaagtgtagcccatggtatttattttcagtcaaaccaaagttacatataattctgcctctgcttatacgggatatgaacactaacacactcccttcaaagacttgtgcaggccaaattgttggaatgctgattttcttgacaactctaaaccccaaaactatgataagacgttatggtgtagttgaagatagcatagtcagatgtttgcttaaaacctagagactttatgtgttgcttttcatgtgctgtgccaagtcttgataatactttttcccccaaccaagggacctcataacctgattatggttattgctttacaaacagttttgacagaaggtggctgctagagcttaacaaatgtcccggttccatgtgatggagccggttcttgcaaactaagctcaatgtttatcctttgctgaagtcagcaaatagagttagagagatacccagtcatctatcacaccaaataaaaggacataacggctttcaaagggttttcccacttacccaaaaggctttctgaaagcttctacctctgcaaaaaagaaaaaagaaaaaaaaaaaaaaaaagaaaaaaaggaaaaaaaaacacaagaaaaaaaaagagaaaaaagttagaacaattacggcagattgcatgaaacatgagaacgtcacagtacctgctacttttcattatgtttgtctttgggtcatgatcaacgaaccggaagttattgatgtggtatcaaaggagagattggtattcggaaggatggttctctgatgtctaggatactgctgtagggataatctgaaagggaaacaagtctttcgggtgcatgttcaaaaagctctgatggactggaagtaacttggagagttgttccatcgggagggtggcctaagactacaatgctaaagtatgcatacctcagtttcaaaacttttgaaaggaagtctcagccacagaatgcatatacctgtagagttttgcatgggttttatatgaatacaattttaaaaaatagctgcttgcacattataccagaaaaacctccaaaactgcaattgctttgaaaatagattttaggttttttggagttttctgaaatgcttggtctgtattttgataattgtgcatattatgtaaaaatgttggtggacccataaatgaccagactttttctaagaaaaatgttgctttaatgcatttcatggatttttactcttctatcattgcttgctagtaatagcaaacctgcttttctgcatctgctttgcgtagctattgtaaggctttgaactaatgtatgtatttattgcttgaacttctgtgcataccttataaagcataatgtctgacaatttaaatggctcatgtattcttgcttctaccataagctgattatggggactatgatcttttgtatacagcaaattttaactgtagcacaaacatctgtttatgtattggtgggatatacctgttttatttatcttctttgaggtaaactaatttttgatacttttcattactgtgtactatgttcatactttgaattctctgacgttagaagtcatggttgagaattgtaacagctgttatttgttctgtattcatggctttcactgctgaataaaataaaggaccaaacctaggatttgaaagaaaactgcctacctctaacaccagggagttaccagattctagtttacatcattttagtctacaaagacacaattgcttaaacctagtgggcttaagacttatattctatgtgttggattcatggcaccgttgtattgtttgaaaatcaattaaaatttcatgtgactgttacaagtatttggtagaattaccactaactggcttttcttcagatgagtcagataaagagaatcgtcatcagcattctgtgtctacagctgcttgacttcataggaatgcattgctgtgtgattagggaatcgaagaacagccagctaggaatggagctacagaagttcacttaaaacaaatggtcctggactttaatgtccctgggcagattcagttgcaacatccaatagactattttgtaaagttttgaaattgggcctttacatttttttgagaatttgtaagacctcctactactcgtgttaatactaacacactcctggacagcaacatcactggcgtattagttattgacaaaaccttactcttgttgcttcattcattcacgttgcaatgaaaggggctttccccctctccgtctaagaacacttgattaatcttacttaacagaaaagaggggtgtacatttttctttagaattaatgtatgaacagtgtctcacttctgctggatgtaaaaaaaaatgtatatgaaaccatttcattcagttggttacatttctgaagtacaaataaaaaaagatctaagtctttttgacccattt

SEQ ID NO: 10 - protein, 714 aa, Mus musculus
MMQESATETISNSSMNQNGMSTLSSQLDAGSRDGRSSGDTSSEVSTVELLHLQQQQALQAARQLLLQQQTSGLKSPKSSEKQRPLQVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSTTSKASPPITHHSIVNGQSSVLNARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 11 - RNA, 6360 nt, Mus musculus
  source 1..6360: mol_type = mRNA
ctgattttgtgtacgagtgtccacggacgccaaaacaatcacagagctgcctgatttgttttaattaccagcacaaaatgccatcagtgtgggacgtgatcgggcagaggtgtactcacagtagtgtaaatactgctgtaaacagttgcctgatggtggctttgacagtgagctagcttcggagtttttccttctttttagactgtttttccccccccgcactggctttttgaatcttcttaatttttcatctctctaacaaactcctatgaagttgaaaccgggaagtttgctctaacatttccagagaaggtattaagtcatgatgcaggaatctgcgacagagacaataagcaacagttcaatgaatcaaaatggaatgagcactttaagcagccaattagatgctggcagcagggatgggagatcaagtggtgacaccagctctgaagtaagcacagtggagctgctgcatctgcaacaacagcaggctctccaggcagcaagacaacttttgttacagcagcaaaccagtggattgaaatctcctaagagcagtgagaagcagaggccactgcaggtgcccgtgtcggtggccatgatgactccccaagtgatcacccctcagcagatgcagcagattcttcagcagcaggtcctgtctcctcagcagctccaggccctcctccagcagcagcaagctgtgatgttgcagcagcagcaacagcagcagcagcaacagcagcaacagcagcagcagcagcagcaacaacagcagcagcagcagcaacagcaacagcagcagcagcaacagcagcagcaacagcaacagcatcctggaaagcaagcaaaagagcagcagcagcaacagcagcagcagcagttggcggcgcagcagctcgtcttccagcagcagcttctccaaatgcagcagctacagcagcaacaacatctgctcagccttcagcgccagggcctcatctccatcccacccggccaagcagcccttcctgtccagtcgctgcctcaagctggcttaagtcctgctgagattcagcaactatggaaagaagtgactggagtccatagtatggaagacaacggcatcaagcatggagggctagacctcacgactaacaattcctcctcgactacctcctccaccacgtccaaagcatcaccacccatcacacatcattccatagtgaacggacagtcttcagttctgaatgcaaggcgggacagctcatcacatgaggagactggggcctcgcacactctctatggccatggagtctgcaagtggccaggctgtgaaagcatatgtgaagattttggacagtttttgaagcaccttaacaatgagcatgcattggatgaccgaagcactgcccaatgtcgagtgcaaatgcaggtggtacagcagttagaaatacagctttctaaggaacgcgaacgtcttcaagcgatgatgacccacttgcacatgcgaccctcagagcccaaaccatctcccaaacctctaaatctggtgtctagtgtcaccatgtcgaagaacatgctggagacatccccacagagcttacctcaaacccctaccacaccaacggccccagtcaccccgattacccagggaccctctgtaatcaccccagccagtgtgcccaatgtgggagccatacgaaggcgacattcagacaaatacaacatccccatgtcatcagagattgccccaaactacgaattttataaaaacgcagatgttagacctcccttcacttatgcaacactcataaggcaggctatcatggagtcatctgacaggcagttaacacttaatgaaatttacagctggtttacacggacatttgcttacttcaggcggaatgccgccacttggaagaatgcagtgcgtcataatcttagcctgcacaaatgttttgttcgagtagaaaatgttaaaggagcagtgtggactgtggacgaagtcgaataccagaagcgaaggtcacaaaagataacaggaagtccaactttagtaaaaaatatacctacaagtttgggctatggagcagctctaaatgccagtctgcaggctgccttggcagaaagcagcttgcctttgctaagtaaccctggactgatcaataacgcctccagtggcctcctgcaggccgtccacgaagacctcaatggttccctggaccacatcgacagcaatgggaacagcagtccgggttgctcacctcagccacacatacattcaatccatgtcaaggaagaacccgtgattgcagaggatgaagactgtccaatgtcattagtgacaacagccaatcacagtccggagttagaagatgacagagagattgaggaagagcctttatctgaggacctggaatgagaacgaacttgtgacacctcagtgtgaagggacatatcactgaccttcataaccactccacaaccatgaatagttgacaaatttttactgtgactatttattaagcatggataaaggagacagccctaaaggaacttactaagccagccctttgggattcagaaccaacaggcaaattgcttgttttcttctctttctgtccttattttgtttgtttgtttgtttgattttactttctttacttattttctttttttcatttttttaaaggcaaaaaaaaaaactgattttcttgggaaaagagagaaagaactgttctttctataatggctttgcccatttaaaaaatgtggctctcaagggttcatgaaatgactgagtatgaggatccatgtcctatagaaagcaactgcgcctcatatactgccaaaaatagtgttagtttcattaatgtgaattttccagcattcagtagtcgtaatgttagaaacaattgctggtcaagttcaacttgttgctattgtttttaatttgcacaggagtagtatcagaaattagtgtcactgcttgtatctagctgacttttaaacaacagaacattagttttttatgttggtgccaccaactgtaaatgacataagttagttattacaaaacacagtaattagactgttgcaaccatctaaaaccttaggcttccagtctgtgctgttagtgtaaagatgtaaagtgcaatcctaagctaacattatctgtgcaagcaccatagaaacatttgcatatctgcatagatcttacaactgtactctttacctccttgtgataaagctttgtctacctgcaaacacagtcaaaggctacagctgcaaaccaaagccaactctaaccatggccaagagctcaaggacagaagcagccacatgctttggtcagccttctgtaacttcaattagtacaaaggaaccttttccatgaactacctgctgttttctgatgacctctgggatcttttcatttagccctaaacaaagaaacgaatatgacaaaacaaaaacaaaaaaacaacccacaactaagagttcattcagtcacagagtaatcttctgaggccaaaagtccatctaaatgcaatgaagatttgctttcattaaagacagaggtgaggacaaaatctgcagtggaagttatgacactctagaaagcaacaaatgtggatcactgaccaaaacaattatgtacttgatgcaaatgcagattgcgtattgttatatatatagtacattgcgtttttgttttccccatctttttggtggtgagttcatgttgttagaagtacatgtcttgtatggtcttaatctctgttgtgtactatttttttatagtcttaagttataataaaaaaaaacaaaaaacaaaaaacaaaaagtaggaaccaaacataaaaggtctagtaaagccaaaaattaatttcctattgattttaaagtgatctagctgagtttttacactgaaagcaaagattatagcaagtgtagcccatggtatttattttcagtcaaaccaaagttacatataattctgcctctgcttatacgggatatgaacactaacacactcccttcaaagacttgtgcaggccaaattgttggaatgctgattttcttgacaactctaaaccccaaaactatgataagacgttatggtgtagttgaagatagcatagtcagatgtttgcttaaaacctagagactttatgtgttgcttttcatgtgctgtgccaagtcttgataatactttttcccccaaccaagggacctcataacctgattatggttattgctttacaaacagttttgacagaaggtggctgctagagcttaacaaatgtcccggttccatgtgatggagccggttcttgcaaactaagctcaatgtttatcctttgctgaagtcagcaaatagagttagagagatacccagtcatctatcacaccaaataaaaggacataacggctttcaaagggttttcccacttacccaaaaggctttctgaaagcttctacctctgcaaaaaagaaaaaagaaaaaaaaaaaaaaaaagaaaaaaaggaaaaaaaaacacaagaaaaaaaaagagaaaaaagttagaacaattacggcagattgcatgaaacatgagaacgtcacagtacctgctacttttcattatgtttgtctttgggtcatgatcaacgaaccggaagttattgatgtggtatcaaaggagagattggtattcggaaggatggttctctgatgtctaggatactgctgtagggataatctgaaagggaaacaagtctttcgggtgcatgttcaaaaagctctgatggactggaagtaacttggagagttgttccatcgggagggtggcctaagactacaatgctaaagtatgcatacctcagtttcaaaacttttgaaaggaagtctcagccacagaatgcatatacctgtagagttttgcatgggttttatatgaatacaattttaaaaaatagctgcttgcacattataccagaaaaacctccaaaactgcaattgctttgaaaatagattttaggttttttggagttttctgaaatgcttggtctgtattttgataattgtgcatattatgtaaaaatgttggtggacccataaatgaccagactttttctaagaaaaatgttgctttaatgcatttcatggatttttactcttctatcattgcttgctagtaatagcaaacctgcttttctgcatctgctttgcgtagctattgtaaggctttgaactaatgtatgtatttattgcttgaacttctgtgcataccttataaagcataatgtctgacaatttaaatggctcatgtattcttgcttctaccataagctgattatggggactatgatcttttgtatacagcaaattttaactgtagcacaaacatctgtttatgtattggtgggatatacctgttttatttatcttctttgaggtaaactaatttttgatacttttcattactgtgtactatgttcatactttgaattctctgacgttagaagtcatggttgagaattgtaacagctgttatttgttctgtattcatggctttcactgctgaataaaataaaggaccaaacctaggatttgaaagaaaactgcctacctctaacaccagggagttaccagattctagtttacatcattttagtctacaaagacacaattgcttaaacctagtgggcttaagacttatattctatgtgttggattcatggcaccgttgtattgtttgaaaatcaattaaaatttcatgtgactgttacaagtatttggtagaattaccactaactggcttttcttcagatgagtcagataaagagaatcgtcatcagcattctgtgtctacagctgcttgacttcataggaatgcattgctgtgtgattagggaatcgaagaacagccagctaggaatggagctacagaagttcacttaaaacaaatggtcctggactttaatgtccctgggcagattcagttgcaacatccaatagactattttgtaaagttttgaaattgggcctttacatttttttgagaatttgtaagacctcctactactcgtgttaatactaacacactcctggacagcaacatcactggcgtattagttattgacaaaaccttactcttgttgcttcattcattcacgttgcaatgaaaggggctttccccctctccgtctaagaacacttgattaatcttacttaacagaaaagaggggtgtacatttttctttagaattaatgtatgaacagtgtctcacttctgctggatgtaaaaaaaaatgtatatgaaaccatttcattcagttggttacatttctgaagtacaaataaaaaaagatctaagtctttttgacccattt

SEQ ID NO: 12 - protein, 693 aa, Mus musculus
MMQESATETISNSSMNQNGMSTLSSQLDAGSRDGRSSGDTSSEVSTVELLHLQQQQALQAARQLLLQQQTSGLKSPKSSEKQRPLQVPVSVAMMTPQVITPQQMQQILQQQVLSPQQLQALLQQQQAVMLQQQQQQQQQQQQQQQQQQQQQQQQQQQQQQQQQQQQQQQQHPGKQAKEQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSTTSKASPPITHHSIVNGQSSVLNARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 13 - RNA, 6423 nt, Mus musculus
  source 1..6423: mol_type = mRNA
ctgattttgtgtacgagtgtccacggacgccaaaacaatcacagagctgcctgatttgttttaattaccagcacaaaatgccatcagtgtgggacgtgatcgggcagaggtgtactcacagtagtgtaaatactgctgtaaacagttgcctgatggtggctttgacagtgagctagcttcggagtttttccttctttttagactgtttttccccccccgcactggctttttgaatcttcttaatttttcatctctctaacaaactcctatgaagttgaaaccgggaagtttgctctaacatttccagagaaggtattaagtcatgatgcaggaatctgcgacagagacaataagcaacagttcaatgaatcaaaatggaatgagcactttaagcagccaattagatgctggcagcagggatgggagatcaagtggtgacaccagctctgaagtaagcacagtggagctgctgcatctgcaacaacagcaggctctccaggcagcaagacaacttttgttacagcagcaaaccagtggattgaaatctcctaagagcagtgagaagcagaggccactgcaggtgcccgtgtcggtggccatgatgactccccaagtgatcacccctcagcagatgcagcagattcttcagcagcaggtcctgtctcctcagcagctccaggccctcctccagcagcagcaagctgtgatgttgcagcagcaacaactacaagagttttacaagaaacagcaagaacagttacatcttcagcttttgcaacagcagcaacagcagcagcagcaacagcagcaacagcagcagcagcagcagcaacaacagcagcagcagcagcaacagcaacagcagcagcagcaacagcagcagcaacagcaacagcatcctggaaagcaagcaaaagagcagcagcagcaacagcagcagcagcagttggcggcgcagcagctcgtcttccagcagcagcttctccaaatgcagcagctacagcagcaacaacatctgctcagccttcagcgccagggcctcatctccatcccacccggccaagcagcccttcctgtccagtcgctgcctcaagctggcttaagtcctgctgagattcagcaactatggaaagaagtgactggagtccatagtatggaagacaacggcatcaagcatggagggctagacctcacgactaacaattcctcctcgactacctcctccaccacgtccaaagcatcaccacccatcacacatcattccatagtgaacggacagtcttcagttctgaatgcaaggcgggacagctcatcacatgaggagactggggcctcgcacactctctatggccatggagtctgcaagtggccaggctgtgaaagcatatgtgaagattttggacagtttttgaagcaccttaacaatgagcatgcattggatgaccgaagcactgcccaatgtcgagtgcaaatgcaggtggtacagcagttagaaatacagctttctaaggaacgcgaacgtcttcaagcgatgatgacccacttgcacatgcgaccctcagagcccaaaccatctcccaaacctctaaatctggtgtctagtgtcaccatgtcgaagaacatgctggagacatccccacagagcttacctcaaacccctaccacaccaacggccccagtcaccccgattacccagggaccctctgtaatcaccccagccagtgtgcccaatgtgggagccatacgaaggcgacattcagacaaatacaacatccccatgtcatcagagattgccccaaactacgaattttataaaaacgcagatgttagacctcccttcacttatgcaacactcataaggcaggctatcatggagtcatctgacaggcagttaacacttaatgaaatttacagctggtttacacggacatttgcttacttcaggcggaatgccgccacttggaagaatgcagtgcgtcataatcttagcctgcacaaatgttttgttcgagtagaaaatgttaaaggagcagtgtggactgtggacgaagtcgaataccagaagcgaaggtcacaaaagataacaggaagtccaactttagtaaaaaatatacctacaagtttgggctatggagcagctctaaatgccagtctgcaggctgccttggcagaaagcagcttgcctttgctaagtaaccctggactgatcaataacgcctccagtggcctcctgcaggccgtccacgaagacctcaatggttccctggaccacatcgacagcaatgggaacagcagtccgggttgctcacctcagccacacatacattcaatccatgtcaaggaagaacccgtgattgcagaggatgaagactgtccaatgtcattagtgacaacagccaatcacagtccggagttagaagatgacagagagattgaggaagagcctttatctgaggacctggaatgagaacgaacttgtgacacctcagtgtgaagggacatatcactgaccttcataaccactccacaaccatgaatagttgacaaatttttactgtgactatttattaagcatggataaaggagacagccctaaaggaacttactaagccagccctttgggattcagaaccaacaggcaaattgcttgttttcttctctttctgtccttattttgtttgtttgtttgtttgattttactttctttacttattttctttttttcatttttttaaaggcaaaaaaaaaaactgattttcttgggaaaagagagaaagaactgttctttctataatggctttgcccatttaaaaaatgtggctctcaagggttcatgaaatgactgagtatgaggatccatgtcctatagaaagcaactgcgcctcatatactgccaaaaatagtgttagtttcattaatgtgaattttccagcattcagtagtcgtaatgttagaaacaattgctggtcaagttcaacttgttgctattgtttttaatttgcacaggagtagtatcagaaattagtgtcactgcttgtatctagctgacttttaaacaacagaacattagttttttatgttggtgccaccaactgtaaatgacataagttagttattacaaaacacagtaattagactgttgcaaccatctaaaaccttaggcttccagtctgtgctgttagtgtaaagatgtaaagtgcaatcctaagctaacattatctgtgcaagcaccatagaaacatttgcatatctgcatagatcttacaactgtactctttacctccttgtgataaagctttgtctacctgcaaacacagtcaaaggctacagctgcaaaccaaagccaactctaaccatggccaagagctcaaggacagaagcagccacatgctttggtcagccttctgtaacttcaattagtacaaaggaaccttttccatgaactacctgctgttttctgatgacctctgggatcttttcatttagccctaaacaaagaaacgaatatgacaaaacaaaaacaaaaaaacaacccacaactaagagttcattcagtcacagagtaatcttctgaggccaaaagtccatctaaatgcaatgaagatttgctttcattaaagacagaggtgaggacaaaatctgcagtggaagttatgacactctagaaagcaacaaatgtggatcactgaccaaaacaattatgtacttgatgcaaatgcagattgcgtattgttatatatatagtacattgcgtttttgttttccccatctttttggtggtgagttcatgttgttagaagtacatgtcttgtatggtcttaatctctgttgtgtactatttttttatagtcttaagttataataaaaaaaaacaaaaaacaaaaaacaaaaagtaggaaccaaacataaaaggtctagtaaagccaaaaattaatttcctattgattttaaagtgatctagctgagtttttacactgaaagcaaagattatagcaagtgtagcccatggtatttattttcagtcaaaccaaagttacatataattctgcctctgcttatacgggatatgaacactaacacactcccttcaaagacttgtgcaggccaaattgttggaatgctgattttcttgacaactctaaaccccaaaactatgataagacgttatggtgtagttgaagatagcatagtcagatgtttgcttaaaacctagagactttatgtgttgcttttcatgtgctgtgccaagtcttgataatactttttcccccaaccaagggacctcataacctgattatggttattgctttacaaacagttttgacagaaggtggctgctagagcttaacaaatgtcccggttccatgtgatggagccggttcttgcaaactaagctcaatgtttatcctttgctgaagtcagcaaatagagttagagagatacccagtcatctatcacaccaaataaaaggacataacggctttcaaagggttttcccacttacccaaaaggctttctgaaagcttctacctctgcaaaaaagaaaaaagaaaaaaaaaaaaaaaaagaaaaaaaggaaaaaaaaacacaagaaaaaaaaagagaaaaaagttagaacaattacggcagattgcatgaaacatgagaacgtcacagtacctgctacttttcattatgtttgtctttgggtcatgatcaacgaaccggaagttattgatgtggtatcaaaggagagattggtattcggaaggatggttctctgatgtctaggatactgctgtagggataatctgaaagggaaacaagtctttcgggtgcatgttcaaaaagctctgatggactggaagtaacttggagagttgttccatcgggagggtggcctaagactacaatgctaaagtatgcatacctcagtttcaaaacttttgaaaggaagtctcagccacagaatgcatatacctgtagagttttgcatgggttttatatgaatacaattttaaaaaatagctgcttgcacattataccagaaaaacctccaaaactgcaattgctttgaaaatagattttaggttttttggagttttctgaaatgcttggtctgtattttgataattgtgcatattatgtaaaaatgttggtggacccataaatgaccagactttttctaagaaaaatgttgctttaatgcatttcatggatttttactcttctatcattgcttgctagtaatagcaaacctgcttttctgcatctgctttgcgtagctattgtaaggctttgaactaatgtatgtatttattgcttgaacttctgtgcataccttataaagcataatgtctgacaatttaaatggctcatgtattcttgcttctaccataagctgattatggggactatgatcttttgtatacagcaaattttaactgtagcacaaacatctgtttatgtattggtgggatatacctgttttatttatcttctttgaggtaaactaatttttgatacttttcattactgtgtactatgttcatactttgaattctctgacgttagaagtcatggttgagaattgtaacagctgttatttgttctgtattcatggctttcactgctgaataaaataaaggaccaaacctaggatttgaaagaaaactgcctacctctaacaccagggagttaccagattctagtttacatcattttagtctacaaagacacaattgcttaaacctagtgggcttaagacttatattctatgtgttggattcatggcaccgttgtattgtttgaaaatcaattaaaatttcatgtgactgttacaagtatttggtagaattaccactaactggcttttcttcagatgagtcagataaagagaatcgtcatcagcattctgtgtctacagctgcttgacttcataggaatgcattgctgtgtgattagggaatcgaagaacagccagctaggaatggagctacagaagttcacttaaaacaaatggtcctggactttaatgtccctgggcagattcagttgcaacatccaatagactattttgtaaagttttgaaattgggcctttacatttttttgagaatttgtaagacctcctactactcgtgttaatactaacacactcctggacagcaacatcactggcgtattagttattgacaaaaccttactcttgttgcttcattcattcacgttgcaatgaaaggggctttccccctctccgtctaagaacacttgattaatcttacttaacagaaaagaggggtgtacatttttctttagaattaatgtatgaacagtgtctcacttctgctggatgtaaaaaaaaatgtatatgaaaccatttcattcagttggttacatttctgaagtacaaataaaaaaagatctaagtctttttgacccattt

SEQ ID NO: 14 - protein, 714 aa, Mus musculus; identical to SEQ ID NO: 10

SEQ ID NO: 15 - RNA, 3836 nt, Mus musculus
  source 1..3836: mol_type = mRNA
aggagcgtcgccggagcccggggaaggacgatcggaagctggagagagccaagccatcctccaaagtgcgatgcccccaaggggctaaggcgcgcggcgatcggcgctagaaagatcgcttcggagcgcaggggcagccccggcgagtggcggcgggcccgggctgcgacccgagcgagccccatgccccgtgcgggctgacgggccggagcccgcactgagcggccgggccggacaggtcccgattgagcggcaatgatggtggagtctgcatcggagacaatcaggtcggctccgtctggtcagaacggcgtgggcagcctctctgcgcaggctgatggcggcggtggggccgggaccgcgggcacggccccggctgccggtagggatgcctcaggcagggaagcagccagcggaggagccgacagcaatggcgagatgagccccgcggagcttctgcacttccaacagcaacaggctctccaggtggcccggcagttcctgttgcagcaagcctccagcctgaactccccggggaacaatgacagtaaacagtcggcctctgcggtgcaggtgcctgtgtctgtggccatgatgtcccagcagatgcttaccccccagcagatgcagcagatcctgtcgccacctcagctgcaggccttgctgcagcagcaacaggctctcatgctccagcagctgcaggagtattacaagaagcagcaagagcagctccacctgcagctcctcacccagcagcaggctggaaaacagcagcccaaagaggccctggggaacaagcagctggccttccagcagcagctcctgcagatgcaacagttgcagcaacagcacttgctcaacctccagaggcaggggctggtcagcctgcagccaagccaagcctcagggcccctccaggccctcccgcaagcagccgtgtgcccgacagacctgccccagctgtggaaaggcgagggtgccccggggcagcccgctgaggacagcggcaggcaggaggggctggacctcgccagcacagctgttactgctacctcgtttgccagccctcccaaggtctccccgcccctctcccaccaccccttacccaacggacaacccactgtgctcacatctcggagagacagctcctcccatgaggagacccccagttcccaccctctttatggacatggggagtgcaagtggccaggctgtgagaccctgtgtgaagacctgggccagtttatcaaacacctcaacacagaacacgctttggatgaccggagcacggcccaatgccgcgtgcagatgcaggtggtccaacagctggagatccagctggccaaggagagtgagcggctgcaggccatgatggcacatctgcacatgcggccttcagagcccaagcccttcagccagccactgaacccagtccctggctcctcctcattctccaaggtgaccgtctccgcagacccgttcccggatggcctcgtgcaccccccaacttcagccgctgcccccgtcacacccctgcgtccccctggcctgggctctgcctctttgcacagtgggggccctgcccgcaggagaagtaatgacaaattctgctcccccatctcctcagagcttgcccagaatcatgagttctacaagaacgctgacgtcaggccccccttcacctacgcttcccttatccgccaggccattctggaaactccagatcggcagctgacgctaaatgagatttataactggttcaccaggatgttcgcctacttccgaagaaacacggccacctggaagaatgccgtgcgccacaacctcagcctgcacaagtgtttcgtccgtgtggagaacgtcaagggggccgtgtggactgtggatgagcgggaatatcagaaacggagaccgccaaagatgacagggagtcccactctggtgaagaacatgatctcgggcctcagttacggggcacttaacgccagctaccaggctgccctggcagagagcagcttccctcttctcagcaaccccggcatgctgaaccctggctcagccagcagccttctgcccctcagccaggaggacctgggtgtccctggggaaccactacccagcaatgggagcagcagcccgcctcgtctttcccctcctcagtacagccaccagatacaagtcaaagaggagcctgcagaggcagaggaagaccgacggccggggcctcccttgggcgcccccaaccccagcactgtggggcctccagaagacagggacctggaggaggacctgggaggagaggacatgtcctaagggcagggtgggcgggcagggcaggccaagaccctgccgcccagtggaccccggcctacccaccccactctacagcccctccaacctcaaggcacttcggggactcagacgggggagatccgggccagtagctcccatgccacctgactgacagacgcctggggacaggcatatccctgctccccgcagagaacacagaacccttggactgggacccgtagaggacatggagggcccagaccctctcagcccctccctgtattcccaaccaccaccagcagcagcagcagggctctccttccccaccagtgctcccctccaagaccccttagcacaaggggagacccgcaggcggggctgagccacccccacaaccctaccttcttcatcccctctgtcccgtctgtggcagaggtttctggccagaggacccttcccggttcatctcccctcatcttcttttcccaactgtgaaaattagttctccggtcccagcttcctgctcaaaggggcaagcaggctgggggcggggcacctgcagaccagccccctcagctgcgggagcggagcaagttgtgccctgcccttccctccctgacctgaggcagaacccagattaagcaggtctccaccccaccctgtttgctgggcactcctgcggcaccccgagagggacccccatgctggacaccttcagagcgtgtgcataggtggtctctgacctgaacatcacagtgcggagaaagcccggctggaaacaccctcgcccccagcctacgggtccccgaaatggggtcctctcaccccgccccctcacctcctcctgtccttccctctcccagttaaaacggatgaccaaagacctttcttatgccggaagcaaaaaccaaaactttttgttggctttttcttttgccgcctcccgagcaccctgccctcccaaccctgccccctgatcccgggctggaagccttccctccttcacttaagttattgtgttaaaccaaagtttacagtgtctgttggtagcaagaccccctcctcccctctaccaccccccccaagggtccaggggtcctactcgcttcccccctgctcataccttgcctatggagacatgagagggcagaatcctgcccccagccctgggctgctttcgggggtcgggggagggctcttcagatgcctccataggaccaagtccccatcattgccgggagtgtggattctagcagcagagctggaaacgatgagattccacagaccccctatgggggacccacaacccaagaccgcagactgggtgtaagggatgcccggaacaccctgaccaggcccctcccctaagacttcttggatatttcccaggagaatcccacattcaccgccaaacccctatccctacccaacacaagctgccctcctgaaaggcaagggcgccccgcccccggccttcccccatgtacccccacctgtgttccgtttgaccagcacagaaatattaaagtcctctatttaccggg

SEQ ID NO: 16 - protein, 685 aa, Mus musculus
MMVESASETIRSAPSGQNGVGSLSAQADGGGGAGTAGTAPAAGRDASGREAASGGADSNGEMSPAELLHFQQQQALQVARQFLLQQASSLNSPGNNDSKQSASAVQVPVSVAMMSQQMLTPQQMQQILSPPQLQALLQQQQALMLQQLQEYYKKQQEQLHLQLLTQQQAGKQQPKEALGNKQLAFQQQLLQMQQLQQQHLLNLQRQGLVSLQPSQASGPLQALPQAAVCPTDLPQLWKGEGAPGQPAEDSGRQEGLDLASTAVTATSFASPPKVSPPLSHHPLPNGQPTVLTSRRDSSSHEETPSSHPLYGHGECKWPGCETLCEDLGQFIKHLNTEHALDDRSTAQCRVQMQVVQQLEIQLAKESERLQAMMAHLHMRPSEPKPFSQPLNPVPGSSSFSKVTVSADPFPDGLVHPPTSAAAPVTPLRPPGLGSASLHSGGPARRRSNDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLSNPGMLNPGSASSLLPLSQEDLGVPGEPLPSNGSSSPPRLSPPQYSHQIQVKEEPAEAEEDRRPGPPLGAPNPSTVGPPEDRDLEEDLGGEDMS

SEQ ID NO: 17 - RNA, 3800 nt, Mus musculus
  source 1..3800: mol_type = mRNA
aggagcgtcgccggagcccggggaaggacgatcggaagctggagagagccaagccatcctccaaagtgcgatgcccccaaggggctaaggcgcgcggcgatcggcgctagaaagatcgcttcggagcgcaggggcagccccggcgagtggcggcgggcccgggctgcgacccgagcgagccccatgccccgtgcgggctgacgggccggagcccgcactgagcggccgggccggacaggtcccgattgagcggcaatgatggtggagtctgcatcggagacaatcaggtcggctccgtctggtcagaacggcgtgggcagcctctctgcgcaggctgatggcggcggtggggccgggaccgcgggcacggccccggctgccggtagggatgcctcaggcagggaagcagccagcggaggagccgacagcaatggcgagatgagccccgcggagcttctgcacttccaacagcaacaggctctccaggtggcccggcagttcctgttgcagcaagcctccagcctgaactccccggggaacaatgacagtaaacagtcggcctctgcggtgcaggtgcctgtgtctgtggccatgatgtcccagcagatgcttaccccccagcagatgcagcagatcctgtcgccacctcagctgcaggccttgctgcagcagcaacaggctctcatgctccagcagctgcaggagtattacaagaagcagcaagagcagctccacctgcagctcctcacccagcagcaggctggaaaacagcagcccaaagaggccctggggaacaagcagctggccttccagcagcagctcctgcagatgcaacagttgcagcaacagcacttgctcaacctccagaggcaggggctggtcagcctgcagccaagccaagcctcagggcccctccaggccctcccgcaagcagccgtgtgcccgacagacctgccccagctgtggaaaggcgagggtgccccggggcagcccgctgaggacagcggcaggcaggaggggctggacctcgccagcacagctgttactgctacctcgtttgccagccctcccaaggtctccccgcccctctcccaccaccccttacccaacggacaacccactgtgctcacatctcggagagacagctcctcccatgaggagacccccagttcccaccctctttatggacatggggagtgcaagtggccaggctgtgagaccctgtgtgaagacctgggccagtttatcaaacacctcaacacagaacacgctttggatgaccggagcacggcccaatgccgcgtgcagatgcaggtggtccaacagctggagatccagctggccaaggagagtgagcggctgcaggccatgatggcacatctgcacatgcggccttcagagcccaagcccttcagccagccagtgaccgtctccgcagacccgttcccggatggcctcgtgcaccccccaacttcagccgctgcccccgtcacacccctgcgtccccctggcctgggctctgcctctttgcacagtgggggccctgcccgcaggagaagtaatgacaaattctgctcccccatctcctcagagcttgcccagaatcatgagttctacaagaacgctgacgtcaggccccccttcacctacgcttcccttatccgccaggccattctggaaactccagatcggcagctgacgctaaatgagatttataactggttcaccaggatgttcgcctacttccgaagaaacacggccacctggaagaatgccgtgcgccacaacctcagcctgcacaagtgtttcgtccgtgtggagaacgtcaagggggccgtgtggactgtggatgagcgggaatatcagaaacggagaccgccaaagatgacagggagtcccactctggtgaagaacatgatctcgggcctcagttacggggcacttaacgccagctaccaggctgccctggcagagagcagcttccctcttctcagcaaccccggcatgctgaaccctggctcagccagcagccttctgcccctcagccaggaggacctgggtgtccctggggaaccactacccagcaatgggagcagcagcccgcctcgtctttcccctcctcagtacagccaccagatacaagtcaaagaggagcctgcagaggcagaggaagaccgacggccggggcctcccttgggcgcccccaaccccagcactgtggggcctccagaagacagggacctggaggaggacctgggaggagaggacatgtcctaagggcagggtgggcgggcagggcaggccaagaccctgccgcccagtggaccccggcctacccaccccactctacagcccctccaacctcaaggcacttcggggactcagacgggggagatccgggccagtagctcccatgccacctgactgacagacgcctggggacaggcatatccctgctccccgcagagaacacagaacccttggactgggacccgtagaggacatggagggcccagaccctctcagcccctccctgtattcccaaccaccaccagcagcagcagcagggctctccttccccaccagtgctcccctccaagaccccttagcacaaggggagacccgcaggcggggctgagccacccccacaaccctaccttcttcatcccctctgtcccgtctgtggcagaggtttctggccagaggacccttcccggttcatctcccctcatcttcttttcccaactgtgaaaattagttctccggtcccagcttcctgctcaaaggggcaagcaggctgggggcggggcacctgcagaccagccccctcagctgcgggagcggagcaagttgtgccctgcccttccctccctgacctgaggcagaacccagattaagcaggtctccaccccaccctgtttgctgggcactcctgcggcaccccgagagggacccccatgctggacaccttcagagcgtgtgcataggtggtctctgacctgaacatcacagtgcggagaaagcccggctggaaacaccctcgcccccagcctacgggtccccgaaatggggtcctctcaccccgccccctcacctcctcctgtccttccctctcccagttaaaacggatgaccaaagacctttcttatgccggaagcaaaaaccaaaactttttgttggctttttcttttgccgcctcccgagcaccctgccctcccaaccctgccccctgatcccgggctggaagccttccctccttcacttaagttattgtgttaaaccaaagtttacagtgtctgttggtagcaagaccccctcctcccctctaccaccccccccaagggtccaggggtcctactcgcttcccccctgctcataccttgcctatggagacatgagagggcagaatcctgcccccagccctgggctgctttcgggggtcgggggagggctcttcagatgcctccataggaccaagtccccatcattgccgggagtgtggattctagcagcagagctggaaacgatgagattccacagaccccctatgggggacccacaacccaagaccgcagactgggtgtaagggatgcccggaacaccctgaccaggcccctcccctaagacttcttggatatttcccaggagaatcccacattcaccgccaaacccctatccctacccaacacaagctgccctcctgaaaggcaagggcgccccgcccccggccttcccccatgtacccccacctgtgttccgtttgaccagcacagaaatattaaagtcctctatttaccggg

SEQ ID NO: 18 - protein, 673 aa, Mus musculus
MMVESASETIRSAPSGQNGVGSLSAQADGGGGAGTAGTAPAAGRDASGREAASGGADSNGEMSPAELLHFQQQQALQVARQFLLQQASSLNSPGNNDSKQSASAVQVPVSVAMMSQQMLTPQQMQQILSPPQLQALLQQQQALMLQQLQEYYKKQQEQLHLQLLTQQQAGKQQPKEALGNKQLAFQQQLLQMQQLQQQHLLNLQRQGLVSLQPSQASGPLQALPQAAVCPTDLPQLWKGEGAPGQPAEDSGRQEGLDLASTAVTATSFASPPKVSPPLSHHPLPNGQPTVLTSRRDSSSHEETPSSHPLYGHGECKWPGCETLCEDLGQFIKHLNTEHALDDRSTAQCRVQMQVVQQLEIQLAKESERLQAMMAHLHMRPSEPKPFSQPVTVSADPFPDGLVHPPTSAAAPVTPLRPPGLGSASLHSGGPARRRSNDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLSNPGMLNPGSASSLLPLSQEDLGVPGEPLPSNGSSSPPRLSPPQYSHQIQVKEEPAEAEEDRRPGPPLGAPNPSTVGPPEDRDLEEDLGGEDMS

SEQ ID NO: 19 - RNA, 3854 nt, Mus musculus
  source 1..3854: mol_type = mRNA
aggagcgtcgccggagcccggggaaggacgatcggaagctggagagagccaagccatcctccaaagtgcgatgcccccaaggggctaaggcgcgcggcgatcggcgctagaaagatcgcttcggagcgcaggggcagccccggcgagtggcggcgggcccgggctgcgacccgagcgagccccatgccccgtgcgggctgacgggccggagcccgcactgagcggccgggccggacaggtcccgattgagcggcaatgatggtggagtctgcatcggagacaatcaggtcggctccgtctggtcagaacggcgtgggcagcctctctgcgcaggctgatggcggcggtggggccgggaccgcgggcacggccccggctgccggtagggatgcctcaggcagggaagcagccagcggaggagccgacagcaatggcgagatgagccccgcggagcttctgcacttccaacagcaacaggctctccaggtggcccggcagttcctgttgcagcaagcctccagcctgaactccccggggaacaatgacagtaaacagtcggcctctgcggtgcaggtgcctgtgtctgtggccatgatgtcccagcagatgcttaccccccagcagatgcagcagatcctgtcgccacctcagctgcaggccttgctgcagcagcaacaggctctcatgctccagcagctgcaggagtattacaagaagcagcaagagcagctccacctgcagctcctcacccagcagcaggctggaaaacagcagcccaaagaggccctggggaacaagcagctggccttccagcagcagctcctgcagatgcaacagttgcagcaacagcacttgctcaacctccagaggcaggggctggtcagcctgcagccaagccaagcctcagggcccctccaggccctcccgcaagccgtgtgcccgacagacctgccccagctgtggaaaggcgagggtgccccggggcagcccgctgaggacagcggcaggcaggaggggctggacctcgccagcacagctgttactgctacctcgtttgccagccctcccaaggtctccccgcccctctcccaccaccccttacccaacggacaacccactgtgctcacatctcggagagacagctcctcccatgaggagacccccagttcccaccctctttatggacatggggagtgcaagtggccaggctgtgagaccctgtgtgaagacctgggccagtttatcaaacacctcaacacagaacacgctttggatgaccggagcacggcccaatgccgcgtgcagatgcaggtggtccaacagctggagatccagctggccaaggagagtgagcggctgcaggccatgatggcacatctgcacatgcggccttcagagcccaagcccttcagccagccacacctaccccattgctcacagctgaacccagtccctggctcctcctcattctccaaggtgaccgtctccgcagacccgttcccggatggcctcgtgcaccccccaacttcagccgctgcccccgtcacacccctgcgtccccctggcctgggctctgcctctttgcacagtgggggccctgcccgcaggagaagtaatgacaaattctgctcccccatctcctcagagcttgcccagaatcatgagttctacaagaacgctgacgtcaggccccccttcacctacgcttcccttatccgccaggccattctggaaactccagatcggcagctgacgctaaatgagatttataactggttcaccaggatgttcgcctacttccgaagaaacacggccacctggaagaatgccgtgcgccacaacctcagcctgcacaagtgtttcgtccgtgtggagaacgtcaagggggccgtgtggactgtggatgagcgggaatatcagaaacggagaccgccaaagatgacagggagtcccactctggtgaagaacatgatctcgggcctcagttacggggcacttaacgccagctaccaggctgccctggcagagagcagcttccctcttctcagcaaccccggcatgctgaaccctggctcagccagcagccttctgcccctcagccaggaggacctgggtgtccctggggaaccactacccagcaatgggagcagcagcccgcctcgtctttcccctcctcagtacagccaccagatacaagtcaaagaggagcctgcagaggcagaggaagaccgacggccggggcctcccttgggcgcccccaaccccagcactgtggggcctccagaagacagggacctggaggaggacctgggaggagaggacatgtcctaagggcagggtgggcgggcagggcaggccaagaccctgccgcccagtggaccccggcctacccaccccactctacagcccctccaacctcaaggcacttcggggactcagacgggggagatccgggccagtagctcccatgccacctgactgacagacgcctggggacaggcatatccctgctccccgcagagaacacagaacccttggactgggacccgtagaggacatggagggcccagaccctctcagcccctccctgtattcccaaccaccaccagcagcagcagcagggctctccttccccaccagtgctcccctccaagaccccttagcacaaggggagacccgcaggcggggctgagccacccccacaaccctaccttcttcatcccctctgtcccgtctgtggcagaggtttctggccagaggacccttcccggttcatctcccctcatcttcttttcccaactgtgaaaattagttctccggtcccagcttcctgctcaaaggggcaagcaggctgggggcggggcacctgcagaccagccccctcagctgcgggagcggagcaagttgtgccctgcccttccctccctgacctgaggcagaacccagattaagcaggtctccaccccaccctgtttgctgggcactcctgcggcaccccgagagggacccccatgctggacaccttcagagcgtgtgcataggtggtctctgacctgaacatcacagtgcggagaaagcccggctggaaacaccctcgcccccagcctacgggtccccgaaatggggtcctctcaccccgccccctcacctcctcctgtccttccctctcccagttaaaacggatgaccaaagacctttcttatgccggaagcaaaaaccaaaactttttgttggctttttcttttgccgcctcccgagcaccctgccctcccaaccctgccccctgatcccgggctggaagccttccctccttcacttaagttattgtgttaaaccaaagtttacagtgtctgttggtagcaagaccccctcctcccctctaccaccccccccaagggtccaggggtcctactcgcttcccccctgctcataccttgcctatggagacatgagagggcagaatcctgcccccagccctgggctgctttcgggggtcgggggagggctcttcagatgcctccataggaccaagtccccatcattgccgggagtgtggattctagcagcagagctggaaacgatgagattccacagaccccctatgggggacccacaacccaagaccgcagactgggtgtaagggatgcccggaacaccctgaccaggcccctcccctaagacttcttggatatttcccaggagaatcccacattcaccgccaaacccctatccctacccaacacaagctgccctcctgaaaggcaagggcgccccgcccccggccttcccccatgtacccccacctgtgttccgtttgaccagcacagaaatattaaagtcctctatttaccggg

SEQ ID NO: 20 - protein, 691 aa, Mus musculus
MMVESASETIRSAPSGQNGVGSLSAQADGGGGAGTAGTAPAAGRDASGREAASGGADSNGEMSPAELLHFQQQQALQVARQFLLQQASSLNSPGNNDSKQSASAVQVPVSVAMMSQQMLTPQQMQQILSPPQLQALLQQQQALMLQQLQEYYKKQQEQLHLQLLTQQQAGKQQPKEALGNKQLAFQQQLLQMQQLQQQHLLNLQRQGLVSLQPSQASGPLQALPQAVCPTDLPQLWKGEGAPGQPAEDSGRQEGLDLASTAVTATSFASPPKVSPPLSHHPLPNGQPTVLTSRRDSSSHEETPSSHPLYGHGECKWPGCETLCEDLGQFIKHLNTEHALDDRSTAQCRVQMQVVQQLEIQLAKESERLQAMMAHLHMRPSEPKPFSQPHLPHCSQLNPVPGSSSFSKVTVSADPFPDGLVHPPTSAAAPVTPLRPPGLGSASLHSGGPARRRSNDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLSNPGMLNPGSASSLLPLSQEDLGVPGEPLPSNGSSSPPRLSPPQYSHQIQVKEEPAEAEEDRRPGPPLGAPNPSTVGPPEDRDLEEDLGGEDMS

SEQ ID NO: 21 - RNA, 3833 nt, Mus musculus
  source 1..3833: mol_type = mRNA
aggagcgtcgccggagcccggggaaggacgatcggaagctggagagagccaagccatcctccaaagtgcgatgcccccaaggggctaaggcgcgcggcgatcggcgctagaaagatcgcttcggagcgcaggggcagccccggcgagtggcggcgggcccgggctgcgacccgagcgagccccatgccccgtgcgggctgacgggccggagcccgcactgagcggccgggccggacaggtcccgattgagcggcaatgatggtggagtctgcatcggagacaatcaggtcggctccgtctggtcagaacggcgtgggcagcctctctgcgcaggctgatggcggcggtggggccgggaccgcgggcacggccccggctgccggtagggatgcctcaggcagggaagcagccagcggaggagccgacagcaatggcgagatgagccccgcggagcttctgcacttccaacagcaacaggctctccaggtggcccggcagttcctgttgcagcaagcctccagcctgaactccccggggaacaatgacagtaaacagtcggcctctgcggtgcaggtgcctgtgtctgtggccatgatgtcccagcagatgcttaccccccagcagatgcagcagatcctgtcgccacctcagctgcaggccttgctgcagcagcaacaggctctcatgctccagcagctgcaggagtattacaagaagcagcaagagcagctccacctgcagctcctcacccagcagcaggctggaaaacagcagcccaaagaggccctggggaacaagcagctggccttccagcagcagctcctgcagatgcaacagttgcagcaacagcacttgctcaacctccagaggcaggggctggtcagcctgcagccaagccaagcctcagggcccctccaggccctcccgcaagccgtgtgcccgacagacctgccccagctgtggaaaggcgagggtgccccggggcagcccgctgaggacagcggcaggcaggaggggctggacctcgccagcacagctgttactgctacctcgtttgccagccctcccaaggtctccccgcccctctcccaccaccccttacccaacggacaacccactgtgctcacatctcggagagacagctcctcccatgaggagacccccagttcccaccctctttatggacatggggagtgcaagtggccaggctgtgagaccctgtgtgaagacctgggccagtttatcaaacacctcaacacagaacacgctttggatgaccggagcacggcccaatgccgcgtgcagatgcaggtggtccaacagctggagatccagctggccaaggagagtgagcggctgcaggccatgatggcacatctgcacatgcggccttcagagcccaagcccttcagccagccactgaacccagtccctggctcctcctcattctccaaggtgaccgtctccgcagacccgttcccggatggcctcgtgcaccccccaacttcagccgctgcccccgtcacacccctgcgtccccctggcctgggctctgcctctttgcacagtgggggccctgcccgcaggagaagtaatgacaaattctgctcccccatctcctcagagcttgcccagaatcatgagttctacaagaacgctgacgtcaggccccccttcacctacgcttcccttatccgccaggccattctggaaactccagatcggcagctgacgctaaatgagatttataactggttcaccaggatgttcgcctacttccgaagaaacacggccacctggaagaatgccgtgcgccacaacctcagcctgcacaagtgtttcgtccgtgtggagaacgtcaagggggccgtgtggactgtggatgagcgggaatatcagaaacggagaccgccaaagatgacagggagtcccactctggtgaagaacatgatctcgggcctcagttacggggcacttaacgccagctaccaggctgccctggcagagagcagcttccctcttctcagcaaccccggcatgctgaaccctggctcagccagcagccttctgcccctcagccaggaggacctgggtgtccctggggaaccactacccagcaatgggagcagcagcccgcctcgtctttcccctcctcagtacagccaccagatacaagtcaaagaggagcctgcagaggcagaggaagaccgacggccggggcctcccttgggcgcccccaaccccagcactgtggggcctccagaagacagggacctggaggaggacctgggaggagaggacatgtcctaagggcagggtgggcgggcagggcaggccaagaccctgccgcccagtggaccccggcctacccaccccactctacagcccctccaacctcaaggcacttcggggactcagacgggggagatccgggccagtagctcccatgccacctgactgacagacgcctggggacaggcatatccctgctccccgcagagaacacagaacccttggactgggacccgtagaggacatggagggcccagaccctctcagcccctccctgtattcccaaccaccaccagcagcagcagcagggctctccttccccaccagtgctcccctccaagaccccttagcacaaggggagacccgcaggcggggctgagccacccccacaaccctaccttcttcatcccctctgtcccgtctgtggcagaggtttctggccagaggacccttcccggttcatctcccctcatcttcttttcccaactgtgaaaattagttctccggtcccagcttcctgctcaaaggggcaagcaggctgggggcggggcacctgcagaccagccccctcagctgcgggagcggagcaagttgtgccctgcccttccctccctgacctgaggcagaacccagattaagcaggtctccaccccaccctgtttgctgggcactcctgcggcaccccgagagggacccccatgctggacaccttcagagcgtgtgcataggtggtctctgacctgaacatcacagtgcggagaaagcccggctggaaacaccctcgcccccagcctacgggtccccgaaatggggtcctctcaccccgccccctcacctcctcctgtccttccctctcccagttaaaacggatgaccaaagacctttcttatgccggaagcaaaaaccaaaactttttgttggctttttcttttgccgcctcccgagcaccctgccctcccaaccctgccccctgatcccgggctggaagccttccctccttcacttaagttattgtgttaaaccaaagtttacagtgtctgttggtagcaagaccccctcctcccctctaccaccccccccaagggtccaggggtcctactcgcttcccccctgctcataccttgcctatggagacatgagagggcagaatcctgcccccagccctgggctgctttcgggggtcgggggagggctcttcagatgcctccataggaccaagtccccatcattgccgggagtgtggattctagcagcagagctggaaacgatgagattccacagaccccctatgggggacccacaacccaagaccgcagactgggtgtaagggatgcccggaacaccctgaccaggcccctcccctaagacttcttggatatttcccaggagaatcccacattcaccgccaaacccctatccctacccaacacaagctgccctcctgaaaggcaagggcgccccgcccccggccttcccccatgtacccccacctgtgttccgtttgaccagcacagaaatattaaagtcctctatttaccggg

SEQ ID NO: 22 - protein, 684 aa, Mus musculus
MMVESASETIRSAPSGQNGVGSLSAQADGGGGAGTAGTAPAAGRDASGREAASGGADSNGEMSPAELLHFQQQQALQVARQFLLQQASSLNSPGNNDSKQSASAVQVPVSVAMMSQQMLTPQQMQQILSPPQLQALLQQQQALMLQQLQEYYKKQQEQLHLQLLTQQQAGKQQPKEALGNKQLAFQQQLLQMQQLQQQHLLNLQRQGLVSLQPSQASGPLQALPQAVCPTDLPQLWKGEGAPGQPAEDSGRQEGLDLASTAVTATSFASPPKVSPPLSHHPLPNGQPTVLTSRRDSSSHEETPSSHPLYGHGECKWPGCETLCEDLGQFIKHLNTEHALDDRSTAQCRVQMQVVQQLEIQLAKESERLQAMMAHLHMRPSEPKPFSQPLNPVPGSSSFSKVTVSADPFPDGLVHPPTSAAAPVTPLRPPGLGSASLHSGGPARRRSNDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLSNPGMLNPGSASSLLPLSQEDLGVPGEPLPSNGSSSPPRLSPPQYSHQIQVKEEPAEAEEDRRPGPPLGAPNPSTVGPPEDRDLEEDLGGEDMS

SEQ ID NO: 23 - RNA, 3797 nt, Mus musculus
  source 1..3797: mol_type = mRNA
aggagcgtcgccggagcccggggaaggacgatcggaagctggagagagccaagccatcctccaaagtgcgatgcccccaaggggctaaggcgcgcggcgatcggcgctagaaagatcgcttcggagcgcaggggcagccccggcgagtggcggcgggcccgggctgcgacccgagcgagccccatgccccgtgcgggctgacgggccggagcccgcactgagcggccgggccggacaggtcccgattgagcggcaatgatggtggagtctgcatcggagacaatcaggtcggctccgtctggtcagaacggcgtgggcagcctctctgcgcaggctgatggcggcggtggggccgggaccgcgggcacggccccggctgccggtagggatgcctcaggcagggaagcagccagcggaggagccgacagcaatggcgagatgagccccgcggagcttctgcacttccaacagcaacaggctctccaggtggcccggcagttcctgttgcagcaagcctccagcctgaactccccggggaacaatgacagtaaacagtcggcctctgcggtgcaggtgcctgtgtctgtggccatgatgtcccagcagatgcttaccccccagcagatgcagcagatcctgtcgccacctcagctgcaggccttgctgcagcagcaacaggctctcatgctccagcagctgcaggagtattacaagaagcagcaagagcagctccacctgcagctcctcacccagcagcaggctggaaaacagcagcccaaagaggccctggggaacaagcagctggccttccagcagcagctcctgcagatgcaacagttgcagcaacagcacttgctcaacctccagaggcaggggctggtcagcctgcagccaagccaagcctcagggcccctccaggccctcccgcaagccgtgtgcccgacagacctgccccagctgtggaaaggcgagggtgccccggggcagcccgctgaggacagcggcaggcaggaggggctggacctcgccagcacagctgttactgctacctcgtttgccagccctcccaaggtctccccgcccctctcccaccaccccttacccaacggacaacccactgtgctcacatctcggagagacagctcctcccatgaggagacccccagttcccaccctctttatggacatggggagtgcaagtggccaggctgtgagaccctgtgtgaagacctgggccagtttatcaaacacctcaacacagaacacgctttggatgaccggagcacggcccaatgccgcgtgcagatgcaggtggtccaacagctggagatccagctggccaaggagagtgagcggctgcaggccatgatggcacatctgcacatgcggccttcagagcccaagcccttcagccagccagtgaccgtctccgcagacccgttcccggatggcctcgtgcaccccccaacttcagccgctgcccccgtcacacccctgcgtccccctggcctgggctctgcctctttgcacagtgggggccctgcccgcaggagaagtaatgacaaattctgctcccccatctcctcagagcttgcccagaatcatgagttctacaagaacgctgacgtcaggccccccttcacctacgcttcccttatccgccaggccattctggaaactccagatcggcagctgacgctaaatgagatttataactggttcaccaggatgttcgcctacttccgaagaaacacggccacctggaagaatgccgtgcgccacaacctcagcctgcacaagtgtttcgtccgtgtggagaacgtcaagggggccgtgtggactgtggatgagcgggaatatcagaaacggagaccgccaaagatgacagggagtcccactctggtgaagaacatgatctcgggcctcagttacggggcacttaacgccagctaccaggctgccctggcagagagcagcttccctcttctcagcaaccccggcatgctgaaccctggctcagccagcagccttctgcccctcagccaggaggacctgggtgtccctggggaaccactacccagcaatgggagcagcagcccgcctcgtctttcccctcctcagtacagccaccagatacaagtcaaagaggagcctgcagaggcagaggaagaccgacggccggggcctcccttgggcgcccccaaccccagcactgtggggcctccagaagacagggacctggaggaggacctgggaggagaggacatgtcctaagggcagggtgggcgggcagggcaggccaagaccctgccgcccagtggaccccggcctacccaccccactctacagcccctccaacctcaaggcacttcggggactcagacgggggagatccgggccagtagctcccatgccacctgactgacagacgcctggggacaggcatatccctgctccccgcagagaacacagaacccttggactgggacccgtagaggacatggagggcccagaccctctcagcccctccctgtattcccaaccaccaccagcagcagcagcagggctctccttccccaccagtgctcccctccaagaccccttagcacaaggggagacccgcaggcggggctgagccacccccacaaccctaccttcttcatcccctctgtcccgtctgtggcagaggtttctggccagaggacccttcccggttcatctcccctcatcttcttttcccaactgtgaaaattagttctccggtcccagcttcctgctcaaaggggcaagcaggctgggggcggggcacctgcagaccagccccctcagctgcgggagcggagcaagttgtgccctgcccttccctccctgacctgaggcagaacccagattaagcaggtctccaccccaccctgtttgctgggcactcctgcggcaccccgagagggacccccatgctggacaccttcagagcgtgtgcataggtggtctctgacctgaacatcacagtgcggagaaagcccggctggaaacaccctcgcccccagcctacgggtccccgaaatggggtcctctcaccccgccccctcacctcctcctgtccttccctctcccagttaaaacggatgaccaaagacctttcttatgccggaagcaaaaaccaaaactttttgttggctttttcttttgccgcctcccgagcaccctgccctcccaaccctgccccctgatcccgggctggaagccttccctccttcacttaagttattgtgttaaaccaaagtttacagtgtctgttggtagcaagaccccctcctcccctctaccaccccccccaagggtccaggggtcctactcgcttcccccctgctcataccttgcctatggagacatgagagggcagaatcctgcccccagccctgggctgctttcgggggtcgggggagggctcttcagatgcctccataggaccaagtccccatcattgccgggagtgtggattctagcagcagagctggaaacgatgagattccacagaccccctatgggggacccacaacccaagaccgcagactgggtgtaagggatgcccggaacaccctgaccaggcccctcccctaagacttcttggatatttcccaggagaatcccacattcaccgccaaacccctatccctacccaacacaagctgccctcctgaaaggcaagggcgccccgcccccggccttcccccatgtacccccacctgtgttccgtttgaccagcacagaaatattaaagtcctctatttaccggg

SEQ ID NO: 24 - protein, 672 aa, Mus musculus
MMVESASETIRSAPSGQNGVGSLSAQADGGGGAGTAGTAPAAGRDASGREAASGGADSNGEMSPAELLHFQQQQALQVARQFLLQQASSLNSPGNNDSKQSASAVQVPVSVAMMSQQMLTPQQMQQILSPPQLQALLQQQQALMLQQLQEYYKKQQEQLHLQLLTQQQAGKQQPKEALGNKQLAFQQQLLQMQQLQQQHLLNLQRQGLVSLQPSQASGPLQALPQAVCPTDLPQLWKGEGAPGQPAEDSGRQEGLDLASTAVTATSFASPPKVSPPLSHHPLPNGQPTVLTSRRDSSSHEETPSSHPLYGHGECKWPGCETLCEDLGQFIKHLNTEHALDDRSTAQCRVQMQVVQQLEIQLAKESERLQAMMAHLHMRPSEPKPFSQPVTVSADPFPDGLVHPPTSAAAPVTPLRPPGLGSASLHSGGPARRRSNDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLSNPGMLNPGSASSLLPLSQEDLGVPGEPLPSNGSSSPPRLSPPQYSHQIQVKEEPAEAEEDRRPGPPLGAPNPSTVGPPEDRDLEEDLGGEDMS

SEQ ID NO: 25 - RNA, 6510 nt, Homo sapiens
  source 1..6510: mol_type = mRNA
gggtacaaatcagtctagagacttagaagcatttattaacagtgaggctgctgaaggtttttaataacaaaattacaaatactctatctaattggtaagacgtgaccttttgaggtgactataactgaagattgctttacagaagccaaaaaaggtttttgagtcatgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 26 - protein, 601 aa, Homo sapiens
MMQESGTETKSNGSAIQNGSGGSNHLLECGGLREGRSNGETPAVDIGAADLAHAQQQQQQALQVARQLLLQQQQQQQVSGLKSPKRNDKQPALQQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 27 - RNA, 7004 nt, Homo sapiens
  source 1..7004: mol_type = mRNA
atcacagcttggtgctggggctcagcatggccaggtggctaaccagggcttggtgtcaactacatttgggatgatgacacttggaggacacagagccactgtgcagcaggagtcgccactgctaacagccagagtgtcccagaagcttttgttgacaattccagtttccgaacaaaacatttcggcaatggtgagggcttcgatcccttctctgatttgctgtcagccatgaacggatggatgtgatgcctgctagccaaaaggcttccctctgtgtgttgcagtcctgtggcattatgcatgccccctcccagtgaccccaggctttttatggctgtgagacacgttaaaatttcaggggtaagacgtgaccttttgaggtgactataactgaagattgctttacagaagccaaaaaaggtttttgagtcatgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 28 - protein, 677 aa, Homo sapiens
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 29 - RNA, 7177 nt, Homo sapiens
  source 1..7177: mol_type = mRNA
gtactcgcggcggcccggccctcgcccgggagtgcgtgtgtgcgcgcgtgtgtgcgcgagtgagtgtgtgcgcggggtgcgcgcgcgggcgggagtgggcgcgcgcggggaaaggcccgaaaacaccttagtttgcaccgagagaccatttgcagcggaacccgaaagtttgtaagaggaagagagcgcgcggcgagcgagcgagcgggccgggggcagcggcagcggcgccggggaccatggtgctgccggcgcctcctccgcgggcgtgaaggcggcgctcctactccctccccggactccgcggtgtcccagaagcttttgttgacaattccagtttccgaacaaaacatttcggcaatggtgagggcttcgatcccttctctgatttgctgtcagccatgaacggatggatgtgatgcctgctagccaaaaggcttccctctgtgtgttgcagtcctgtggcattatgcatgccccctcccagtgaccccaggctttttatggctgtgagacacgttaaaatttcaggggtaagacgtgaccttttgaggtgactataactgaagattgctttacagaagccaaaaaaggtttttgagtcatgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 30 - protein, 677 aa, Homo sapiens; identical to SEQ ID NO: 28

SEQ ID NO: 31 - RNA, 7174 nt, Homo sapiens
  source 1..7174: mol_type = mRNA
gtactcgcggcggcccggccctcgcccgggagtgcgtgtgtgcgcgcgtgtgtgcgcgagtgagtgtgtgcgcggggtgcgcgcgcgggcgggagtgggcgcgcgcggggaaaggcccgaaaacaccttagtttgcaccgagagaccatttgcagcggaacccgaaagtttgtaagaggaagagagcgcgcggcgagcgagcgagcgggccgggggcagcggcagcggcgccggggaccatggtgctgccggcgcctcctccgcgggcgtgaaggcggcgctcctactccctccccggactccgcggtgtcccagaagcttttgttgacaattccagtttccgaacaaaacatttcggcaatggtgagggcttcgatcccttctctgatttgctgtcagccatgaacggatggatgtgatgcctgctagccaaaaggcttccctctgtgtgttgcagtcctgtggcattatgcatgccccctcccagtgaccccaggctttttatggctgtgagacacgttaaaatttcaggggtaagacgtgaccttttgaggtgactataactgaagattgctttacagaagccaaaaaaggtttttgagtcatgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 32 - protein, 676 aa, Homo sapiens
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSDIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 33 - RNA, 7174 nt, Homo sapiens
  source 1..7174: mol_type = mRNA
gtactcgcggcggcccggccctcgcccgggagtgcgtgtgtgcgcgcgtgtgtgcgcgagtgagtgtgtgcgcggggtgcgcgcgcgggcgggagtgggcgcgcgcggggaaaggcccgaaaacaccttagtttgcaccgagagaccatttgcagcggaacccgaaagtttgtaagaggaagagagcgcgcggcgagcgagcgagcgggccgggggcagcggcagcggcgccggggaccatggtgctgccggcgcctcctccgcgggcgtgaaggcggcgctcctactccctccccggactccgcggtgtcccagaagcttttgttgacaattccagtttccgaacaaaacatttcggcaatggtgagggcttcgatcccttctctgatttgctgtcagccatgaacggatggatgtgatgcctgctagccaaaaggcttccctctgtgtgttgcagtcctgtggcattatgcatgccccctcccagtgaccccaggctttttatggctgtgagacacgttaaaatttcaggggtaagacgtgaccttttgaggtgactataactgaagattgctttacagaagccaaaaaaggtttttgagtcatgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 34 - protein, 676 aa, Homo sapiens
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 35 - RNA, 7082 nt, Homo sapiens
  source 1..7082: mol_type = mRNA
gtactcgcggcggcccggccctcgcccgggagtgcgtgtgtgcgcgcgtgtgtgcgcgagtgagtgtgtgcgcggggtgcgcgcgcgggcgggagtgggcgcgcgcggggaaaggcccgaaaacaccttagtttgcaccgagagaccatttgcagcggaacccgaaagtttgtaagaggaagagagcgcgcggcgagcgagcgagcgggccgggggcagcggcagcggcgccggggaccatggtgctgccggcgcctcctccgcgggcgtgaaggcggcgctcctactccctccccggactccgcggtgtcccagaagcttttgttgacaattccagtttccgaacaaaacatttcggcaatggtgagggcttcgatcccttctctgatttgctgtcagccatgaacggatggatgtgatgcctgctagccaaaaggggtaagacgtgaccttttgaggtgactataactgaagattgctttacagaagccaaaaaaggtttttgagtcatgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 36 - protein, 677 aa, Homo sapiens; identical to SEQ ID NO: 28

SEQ ID NO: 37 - RNA, 7088 nt, Homo sapiens
  source 1..7088: mol_type = mRNA
gccagcgccccggcgaacggcaaagagggagccgctcccgctcggggggccgctggagtgcccagcgggaacccgaaagtttgtaagaggaagagagcgcgcggcgagcgagcgagcgggccgggggcagcggcagcggcgccggggaccatggtgctgccggcgcctcctccgcgggcgtgaaggcggcgctcctactccctccccggactccgcggtgtcccagaagcttttgttgacaattccagtttccgaacaaaacatttcggcaatggtgagggcttcgatcccttctctgatttgctgtcagccatgaacggatggatgtgatgcctgctagccaaaaggcttccctctgtgtgttgcagtcctgtggcattatgcatgccccctcccagtgaccccaggctttttatggctgtgagacacgttaaaatttcaggggtaagacgtgaccttttgaggtgactataactgaagattgctttacagaagccaaaaaaggtttttgagtcatgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 38 - protein, 677 aa, Homo sapiens; identical to SEQ ID NO: 28

SEQ ID NO: 39 - RNA, 7136 nt, Homo sapiens
  source 1..7136: mol_type = mRNA
gccagcgccccggcgaacggcaaagagggagccgctcccgctcggggggccgctggagtgcccagcgggaacccgaaagtttgtaagaggaagagagcgcgcggcgagcgagcgagcgggccgggggcagcggcagcggcgccggggaccatggtgctgccggcgcctcctccgcgggcgtgaaggcggcgctcctactccctccccggactccgcggtgtcccagaagcttttgttgacaattccagtttccgaacaaaacatttcggcaatggtgagggcttcgatcccttctctgatttgctgtcagccatgaacggatggatgtgatgcctgctagccaaaaggcttccctctgtgtgttgcagtcctgtggcattatgcatgccccctcccagtgaccccaggctttttatggctgtgagacacgttaaaatttcaggggtaagacgtgaccttttgaggtgactataactgaagattgctttacagaagccaaaaaaggtttttgagtcatgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagggtgccattcgcaccaacctctcactgcataagtgctttatcagagtagaggatgagtttgggtccttttggacagttgatgatgaagagtttaaacgtggccgccatattcaacgaggccgtcctcgcaaatactgccccgatgaaaactttgacgagcttgtcgcacataacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 40 - protein, 693 aa, Homo sapiens
MMQESGTETKSNGSAIQNGSGGSNHLLECGGLREGRSNGETPAVDIGAADLAHAQQQQQQALQVARQLLLQQQQQQQVSGLKSPKRNDKQPALQVPVSVAMMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKGAIRTNLSLHKCFIRVEDEFGSFWTVDDEEFKRGRHIQRGRPRKYCPDENFDELVAHNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 41 - RNA, 6392 nt, Homo sapiens
  source 1..6392: mol_type = mRNA
gcagtccgcctgcacgccgaggccgccggaccccacgcccggcccgcgcgggcgccccgagcccggctccgcgcgcctggcggctacatggagagtgtcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 42 - protein, 577 aa, Homo sapiens
MMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 43 - RNA, 6389 nt, Homo sapiens
  source 1..6389: mol_type = mRNA
gcagtccgcctgcacgccgaggccgccggaccccacgcccggcccgcgcgggcgccccgagcccggctccgcgcgcctggcggctacatggagagtgtcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 44 - protein, 576 aa, Homo sapiens
MMTPQVITPQQMQQILQQQVLSPQQLQVLLQQQQALMLQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSADIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 45 - RNA, 6482 nt, Homo sapiens
  source 1..6482: mol_type = mRNA
agaaacagccagaacccatctacagcaagaagacggaaatccaaaggcagacagtacgggctcccttcgccaaactcttcattttctctgcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 46 - protein, 577 aa, Homo sapiens; identical to SEQ ID NO: 42

SEQ ID NO: 47 - RNA, 6479 nt, Homo sapiens
  source 1..6479: mol_type = mRNA
agaaacagccagaacccatctacagcaagaagacggaaatccaaaggcagacagtacgggctcccttcgccaaactcttcattttctctgcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 48 - protein, 576 aa, Homo sapiens
MMTPQVITPQQMQQILQQQVLSPQQLQVLLQQQQALMLQQQQLQEFYKKQQEQLQLQLLQQQHAGKQPKEQQQVATQQLAFQQQLLQMQQLQQQHLLSLQRQGLLTIQPGQPALPLQPLAQGMIPTELQQLWKEVTSAHTAEETTGNNHSSLDLTTTCVSSSAPSKTSLIMNPHASTNGQLSVHTPKRESLSHEEHPHSHPLYGHGVCKWPGCEAVCEDFQSFLKHLNSEHALDDRSTAQCRVQMQVVQQLELQLAKDKERLQAMMTHLHVKSTEPKAAPQPLNLVSSVTLSKSASEASPQSLPHTPTTPTAPLTPVTQGPSVITTTSMHTVGPIRRRYSDKYNVPISSDIAQNQEFYKNAEVRPPFTYASLIRQAILESPEKQLTLNEIYNWFTRMFAYFRRNAATWKNAVRHNLSLHKCFVRVENVKGAVWTVDEVEFQKRRPQKISGNPSLIKNMQSSHAYCTPLNAALQASMAENSIPLYTTASMGNPTLGNLASAIREELNGAMEHTNSNESDSSPGRSPMQAVHPVHVKEEPLDPEEAEGPLSLVTTANHSPDFDHDRDYEDEPVNEDME

SEQ ID NO: 49 - RNA, 6479 nt, Homo sapiens
  source 1..6479: mol_type = mRNA
agaaacagccagaacccatctacagcaagaagacggaaatccaaaggcagacagtacgggctcccttcgccaaactcttcattttctctgcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 50 - protein, 576 aa, Homo sapiens; identical to SEQ ID NO: 44

SEQ ID NO: 51 - RNA, 6500 nt, Homo sapiens
  source 1..6500: mol_type = mRNA
ctgaccgcccactttcatggcgcccaggctgtgtgccttttgatactgcaacgcagagcagctgcaggggatgctaataaaatgcaggattttggacactgagcaaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 52 - protein, 577 aa, Homo sapiens; identical to SEQ ID NO: 42

SEQ ID NO: 53 - RNA, 6497 nt, Homo sapiens
  source 1..6497: mol_type = mRNA
ctgaccgcccactttcatggcgcccaggctgtgtgccttttgatactgcaacgcagagcagctgcaggggatgctaataaaatgcaggattttggacactgagcaaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 54 - protein, 576 aa, Homo sapiens; identical to SEQ ID NO: 44

SEQ ID NO: 55 - RNA, 6808 nt, Homo sapiens
  source 1..6808: mol_type = mRNA
actgagctctgtatatgcaaacctgtttttatgccatcgcttccccaggctgagctgcgtttgtttggaaatgtaagccactgccttttctttttttcttttttccttttttttttttcccccctcacttcatctgagggctctggattaattccttgagacatatctttgcatacatatctcatgagttatgtttcctggagccttttactaggaacattaaggtttttgagtcatgatgcaagaatctgggactgagacaaaaagtaacggttcagccatccagaatgggtcgggcggcagcaaccacttactagagtgcggcggtcttcgggaggggcggtccaacggagagacgccggccgtggacatcggggcagctgacctcgcccacgcccagcagcagcagcaacaggcacttcaggtggcaagacagctccttcttcagcagcaacagcagcagcaagttagtggattaaaatctcccaagaggaatgacaaacaaccagctcttcaggttcccgtgtcagtggctatgatgacacctcaagttatcactccccagcaaatgcagcagatcctccagcaacaagtgctgagccctcagcagctccaggttctcctccagcagcagcaggccctcatgcttcaacagcagcagcttcaagagttttataaaaaacaacaggaacagttgcagcttcaacttttacaacaacaacatgctggaaaacagcctaaagagcaacagcaggtggctacccagcagttggcttttcagcagcagcttttacagatgcagcagttacagcagcagcacctcctgtctttgcagcgccaaggccttctgacaattcagcccgggcagcctgcccttccccttcaacctcttgctcaaggcatgattccaacagaactgcagcagctctggaaagaagtgacaagtgctcatactgcagaagaaaccacaggcaacaatcacagcagtttggatctgaccacgacatgtgtctcctcctctgcaccttccaagacctccttaataatgaacccacatgcctctaccaatggacagctctcagtccacactcccaaaagggaaagtttgtcccatgaggagcacccccatagccatcctctctatggacatggtgtatgcaagtggccaggctgtgaagcagtgtgcgaagatttccaatcatttctaaaacatctcaacagtgagcatgcgctggacgatagaagtacagcccaatgtagagtacaaatgcaggttgtacagcagttagagctacagcttgcaaaagacaaagaacgcctgcaagccatgatgacccacctgcatgtgaagtctacagaacccaaagccgcccctcagcccttgaatctggtatcaagtgtcactctctccaagtccgcatcggaggcttctccacagagcttacctcatactccaacgaccccaaccgcccccctgactcccgtcacccaaggcccctctgtcatcacaaccaccagcatgcacacggtgggacccatccgcaggcggtactcagacaaatacaacgtgcccatttcgtcagcagatattgcgcagaaccaagaattttataagaacgcagaagttagaccaccatttacatatgcatctttaattaggcaggccattctcgaatctccagaaaagcagctaacactaaatgagatctataactggttcacacgaatgtttgcttacttccgacgcaacgcggccacgtggaagaatgcagtgcgtcataatcttagtcttcacaagtgttttgtgcgagtagaaaacgttaaaggggcagtatggacagtggatgaagtagaattccaaaaacgaaggccacaaaagatcagtggtaacccttcccttattaaaaacatgcagagcagccacgcctactgcacacctctcaatgcagctttacaggcttcaatggctgagaatagtatacctctatacactaccgcttccatgggaaatcccactctgggcaacttagccagcgcaatacgggaagagctgaacggggcaatggagcataccaacagcaacgagagtgacagcagtccaggcagatctcctatgcaagccgtgcatcctgtacacgtcaaagaagagcccctcgatccagaggaagctgaagggcccctgtccttagtgacaacagccaaccacagtccagattttgaccatgacagagattacgaagatgaaccagtaaacgaggacatggagtgactatcggggcgggccaaccccgagaatgaagattggaaaaaggaaaaaaaaaaaaacacgtcaaaagttagcagtgaaattgttctccatttgttgtacagtctggaggattttcactacgttttgacaactctgaaatgtgttaactcttagtgccatcaagaaccccatttgggagtatttttgatttttctactttttgttgaaaaaaggaatttgtactctgtgcattggatggacttgtttggtacttgggattttcctctcttaaccgtcaacatcagtgttgtaaatttgctaaactgattcacttttagcagcagactttgaactgcagtcctgccaacgttggacactgaggacgcccgacagagcttgtgcacctaagctgcagaccaagcctttgcccagaatttaaggattccaatggacgacctatttgcacagtactgcatgttgattatcactgcctttactccttttttttttttttttttttttttttttttgcttccagttgggatggggaaggcctttgtgtgtgtattggggggaggggttaaaaaataattatcccaaactttttaatgtattgctttttttttttttttttttccttctactataccattttaagttctgacctcaggcctccatttgggccgatggcctcttggaggcttaaagttttctgtaccttgtgatgaatgttaataggtgtttttattatacaaagctgaatgtcatttctcgtttgtagctttctgtcactcattccatcttccttcagacatcaccacgtttctctaaagtcagaaaacattccgttttggtctttttcaaaaaggtcccaaatgctgcactctacacatgaaggccctctcacacagacgtgacgtcctgccagaaagagaatgaatgacagaaaaaaaaaagagagacaaactctaggaacaatgccgattcattccacgcagcagtattgggggtggttcgggggaggggtgtttcggattttctttttttcttttcttttctttttttttttttgcagcaaccattaataaatgccaccacattctaccagcacaaggaaacataggcagcactgaaaaaaaaaaaaaagctcatattaattagactgacaatatggccttggaaggctctcccttgtggaaccaagttgccatgggccttgggtgctctgcgataacgggtgtgggttggttttgtttgcaaaatggccaaaaaaaaaaaccggcttccccgagcagctgccctgaaagtaggggtggcggcggcggcgctgagtttatacattagttcagacctacttggtggcattaaactgtttgaatgcaaattcgatttcagattgaacttgttaagggagttaacgagggctgagttcagcaaatgctaaagtgttaatttcaaatatgcaaatttggtactgcagtttgttatgcaatattatatcaccaacccagtatcacaaaaactcatagaagatatcatgtaggccctgggctttgggggggtcccaaacatggtatgcagaaatgtgatggttacaggtcagtacaacctcagtccttagaacccctccacacttcagctctgcacccactttcctgtcatttatttatataggactgtagttttttttagttcgagagcctttcgaagcttaatttatattctttctttgtaccttttttctaaaattaccaaagatattacacaaaggtaaattatgttctctgttttatgctttatctgatgaagccaaatatcctcttattgttgatcaaaggaggcaaaagaatttagaggcaaatgacaagcgataggctattgcaacctgagaaagagaactgctccttcatcgtaaatttagaagaccaagtagataatggaaccaaagttgttacttttttctagtagttatttttcctttttctttttgtgtacctctacagagaccaaaactcattctcttaaagagattttatggggctactgcagataaaaataggacacaatattaaaggagctacagaaggaagggagtcccatctcaaaaaaaaaatgaatgtatgccactgcaattagagtatccaataaaggagacagtttagagtcaggacagaaaagcttccataattgaactagattacataatagtatttctagaaaaagagatatttttagattgtatgccacttttgtttaagaactgtgctgtgatcactgtattaattttggtttatcttggcatatatccttcagtttgtttttatttttaatttttcctttttttccgattaggctttggtcagcatttttcatttaaagaaaagtaacactcccatccactcataagcttggtacaaaaacttctctggcagttacttttgaagcttcactctgctttctgtataaagggcagtctgtggtcacgcaagactttaaaaaaaaaaaaaaaaaaaaaaaaaaaaaaaaaaaaaaaaacttttccaggcagcttcatgatgtgcaggcagtagccagacagggtcatgggaagggggccctgtgcttctaaactgagtggttgctggttagtttggtattcaaaagaggataaaaatctggtagattagttcattctcagcatgtgtagctagacatgagtaaagataacagcatgagaaactgttagtacgcatacctcagttcaaacctttagggaatgattaaaatttaaaaaaaaaacatttcactcagttgcacttagtcgtatgtcttgcatgcttagtctaaagactgtagcaaaaaaaaaaaaaaaagaaaaattagattttacatatctttgcaggtatcacagccttgcagaagaaccaactgaaaaaaaaattctcaggctttacagcaagcaaacttcactatgatttttacaattctgattctgtatcccctgggggttatcccagttgcttctttaggatggggtttattacgttgtacatatatcccgatgtgtctgtgtgaatctttgtcttttttgggggagggcagagggcggttctttttttagatattgttcctaaaaaggaataaatgcatacacctgtttgtcaaaacacctttgctttttgtgcaactgctttatattaacgatactaaaaaaaaatagctttggaaaaaaaactactgtatgtaacggaattgcagaatatgctgcacatgtattttatttagttatccttgctttaagaatattggatgacatttcctgacatgtgggagggagaaactccctaactttttttttctgcttttaaactgtaacatagttgaagatttcttttttctgttctcattgattggagcattttgtacaggttttgtgtgtgtgtgtgtgtgtgtgtgcgcgcgtgcgtgtgtgttaatctgttttttgatacattcctatcccttgtgtttatcctaccactgccttcctggctatcttaaacaagttcatacatttgaaaagaaaaaaaaatgttgtttaaaaaatgttttctcctgctgcagtaaatattttgcatgatgaaattccagggtcacacttttccaagtttatcagtgaagtagtgattaacaatggggagtgtcaaaactattgaacttttgtataaaaaaaaaaaaactttacaaggtgccaagatgtaaagaaaatctgttactttttttttctcaaagaaaagcatacattagggaggtagtcccgtgtgtcagacaaatgcactgtcaggaatgaggatccaacctacctgtccctagagtccgtcttgtaagatgagttaggctgccccttggaccagccacaaaatggaatatcaaggcttatgtacatacgtgaagagttaccaccagtcctgccacctttggacagctctaacaccatccccagcatccagtcagacctagtaaagaaaaccttggattcttaacccaagataggctgtaaatcactagcttttttttcctcatgaaaaaaaatagagttaaaaaatatttcctctcttttccatattccagctgaactccgtttccaaaggcacaaagaagagtgtgcttattcagattttgaatctttttggtaccttttggttaatgacatagcctcctgaaattctggatgtcttcaaagtcagttttgcttctttatcctgaaaatcagatttacaatgctgaaggcatttcttgggcccagtgtagctcacgcaatctctgctacccataagccttgatgaagatgatacagtccggactgtgagcatggtgcttcatgtatatgtgctgccagtaacaagaatttttttgttttgttttgttttgttttgataaggcataaaagaaactcattccttgacatcaactgtaattccatcattccatgtctgcggatacagacaataaaaaaaatgttgtgtagtcagtactaattactgacattataagcattctcaaatgcaataaaaatgctggttgttcacgctggtagtaaaa

SEQ ID NO: 56 - protein, 677 aa, Homo sapiens; identical to SEQ ID NO: 28

SEQ ID NO: 57 - RNA, 6618 nt, Homo sapiens
  source 1..6618: mol_type = mRNA
acagacatgaaagctaaccgaggacttgagagactcaaactggtgcttttgtctctctctctctgtctttctctctctcacacacacactcacacactcacacacatgcacacacacacatacacacacacaaaaatgaagcacttactttagaaagattatggtaagcatgctggctcagtcttgaacctttgtcacccctcacgttgcacaccaaagacataccctagtgattaaatgctgattttgtgtacgattgtccacggacgccaaaacaatcacagagctgcttgatttgttttaattaccagcacaaaatgccatcagtctgggacgtgatcgggcagaggtgtactcacagtagtgtaaatactgctgtaaatagttgtctgatggtggctttgacagtgagctagcttctgagttttcccttctttttatactgttttctgtgctggcttttttgaatcttcctaatttttcatctctttaacaaactcctatgaagttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggaattgcttccagaaacaaaattatgtatctgtggccactcttctggtgatgggcatcctcacaacacatttgcagtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatgagaactgacttgtgaaacctcagcgtgaagggacatatcactgaccttcataaccactccacaaccatgaatatttgacaaatttttactgtgactatttattaagcatggataaaggagacagccctaaaggaacttactaagccagccctttgggattcagtaccaacaggcaaattgcttgttttcttcttcttcttcttctttttttttttttttttagaaaaaaagacaaaaactgattttcttgaaaaaaaaaaatgaactgttctttctataatggctttgcccatttaaaaaatgtggctcttaagggttcatgaaatgactgaatatgaggatacatgtcctgtagaaagcaaatgcgcctcatatactgccaaaaatagtgttagtttcattaatgtgaattttccagcattcagtagttgtaatgttagaaacaattgctggtcaagttcaacttgttgctattgtttttaatttgcacaggagtagtatcagaaattagtgtcactgcttgtatctagctgaattttaaacaacagaacattagttttttatgttggtgccaccaactgtaaatgacataagttagttattacaaaacacagtaattagactgttgcaaccatctaaaaccttaggcttccagtctgtgctgttagtgttaagatgtaaagtgcaatcctaagctaacattatctgtgcaagcaccatagaaacatttgcatatctgcatagatcttacaactgtactctttacctccttgtgataaagctttgtctacctgcaaacacagtcaaaggctacagctgcaaaccaaagccaactctaaccatggccaagagctcaaggacagaagcagccacatgctttggtcagccttctgtaacttcaattagtacaaaggaaccttttccatgaactacctgctgttttctgatgacctctgggatcttttcatttagccctaaacaaagaaacaaatatgacaaaaaccacaactaaaaaatgttaattcagtcacagagtaatcttctgaggccaaaagtccatctaaatgcaatgaagatttgctttcattaaagacagaggtgaggacaaaatccgcagtggaagttatgatatgctagaaagcaacaaatgtggatcactgaccaaaacgattatgtacttgatgcaaatgcagattgcatattgttatatatatagtactttgtgtttttgttttccctcattcagtcagttattttcagtggtgaatacatgttgttagaagatgtcttgtatggtcttaatctttgttgtgtactatttttttatagtcttaagttataatgaaaaaacaaaaagtaggaaccaaacataaaaggtctagtaaagccaaaaattaatttcatattgattttaaagtgatctagctgagtttttacactgaaagcaaagattatagcaattgtagtccatggtatttattttcagtcaaaccaaagttacatataattctgcctctgcttatacgggatattaacactaacaatacactcccttcaaagacttgcacaggccaaattgttggaatgctggttttcttgacaattccaaaccccaaaactatgataatgagttatgatgtagttgaaaatagcatagtcagatgtttgcttaaaacctagaaacttaacatgttgcttttcatgtgctgtgccaagtcttgataatactttttcccccaaccaagggacctcataacctgattatggttattgctttacaaacagttttgacagaaggtggctgctagagcttaacatacgttcccgttccatgtgatggaaccggttcttgcaaactaagctcatcattgattctttgctgaagtcagcaaatagagttagagagatacccagtcatctatcacaccaaataaaaggacataacggctttcaaaagggttttcccacttacccaaaaggctttctgaaagcttctacctctgcaaaaaaaaaaaaagaaaaaaaaaaaaagaaaaacattagaacaattatggcagattgcatgaaacgtgagaacgtcacagtaactgctacttttcattatgtttgtctttgggtcatgatcaacgaaccggaagtttacaatatggtattaaaagaaagatgggtatggtgaaagatggttttcagtcatctaggatcctactgtaaggattatctgaaaggaaaaatgggtctttcaggtgcatgttcaaaaggctttgagggactggaagtaactgcgagagttgtaccatcagaagggtggcctaagactacaatgctaaagtatgcatacctcagttagaaaacttttgaaaggaagtctcagccacagaatgcatatacctgtagagttttgcatgggttttatatgaatacaattttaaaaaatagctgcttgcacattataccagaaaaacctccaaaactgcaattgctttgaaaatagattttaggttttttggagtttcctgaaatgcttggtctgtattttgataattgtgcatattatgtaaaaatgttggtggacccataaatgaccagactttttctaagaaaaatgttgctttaatgcatttcatgaatttttactcttatatcattgcttgctagtaatagcaaatctgcttttctgcatctgctttgcgtagctattgtaaggctttgaactaatgtatgtatttattgcttgaacttctgtgcataccttataaagcataatgtctgacaatttaaatggctcatgtattcttgcttctatcataagctgattatggggactatgatcttttgtatacagcaaattttaaactgtagcacaaacatctgtttatgtattggtggaatatacctgttttatttatcttttttgaggtaaactaatttttgatacttttcattactgtgtactatgttcatactttgaattctctgacgttagaagtcatggttgagaattgtaacagctgttattcgttctgtattcatggctttcactgctgaataaaataaaggaccaaacctaggatttgaaagaaaactgtctacctctaacaccagggagttatcagattttattttacatagttttagtctacaaagacacaattgcttaaacctagtgggcttaaggcttatattctatgtggttggattcgtggcacagttgtactatttgaaaatcaattaaaattttatgtgaatgttacaagtatttggtagaattaccactaactgggttttctttagataactcagatatggagaaaatgtcatcagcattctgtgtctacagctgcttaacttcataagaatgcatttctttgtgattagggaatcgaagaatagtcagctaggaatagagctacagaagtacacttacataaaccatcctggactttaatgtccctgggcagattcagtcgcaaaatccaatatgatattttgtaaagttttcaagttggacatttacatttttgagaattttgagacttcatcttacacatgccagtattaacacacatttggacaatagctttattaagtctataaagctattgaaaggaacatggcttacccttgttatttcactagttcaggttgcaacgaaaggtttttttgtccatgaacacttggcatatcttacttagcaaaaaagaaggatgtacattttactatagaattaatgtatgaacagtgtgtcactgctgttggatgtaaaaatgtatatgaaaccatttcattcacttgattacatttctgaagtataaataaaaaaatctaattctttttgaccca

SEQ ID NO: 58 - protein, 740 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQELLPETKLCICGHSSGDGHPHNTFAVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 59 - RNA, 6540 nt, Homo sapiens
  source 1..6540: mol_type = mRNA
acagacatgaaagctaaccgaggacttgagagactcaaactggtgcttttgtctctctctctctgtctttctctctctcacacacacactcacacactcacacacatgcacacacacacatacacacacacaaaaatgaagcacttactttagaaagattatggtaagcatgctggctcagtcttgaacctttgtcacccctcacgttgcacaccaaagacataccctagtgattaaatgctgattttgtgtacgattgtccacggacgccaaaacaatcacagagctgcttgatttgttttaattaccagcacaaaatgccatcagtctgggacgtgatcgggcagaggtgtactcacagtagtgtaaatactgctgtaaatagttgtctgatggtggctttgacagtgagctagcttctgagttttcccttctttttatactgttttctgtgctggcttttttgaatcttcctaatttttcatctctttaacaaactcctatgaagttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatgagaactgacttgtgaaacctcagcgtgaagggacatatcactgaccttcataaccactccacaaccatgaatatttgacaaatttttactgtgactatttattaagcatggataaaggagacagccctaaaggaacttactaagccagccctttgggattcagtaccaacaggcaaattgcttgttttcttcttcttcttcttctttttttttttttttttagaaaaaaagacaaaaactgattttcttgaaaaaaaaaaatgaactgttctttctataatggctttgcccatttaaaaaatgtggctcttaagggttcatgaaatgactgaatatgaggatacatgtcctgtagaaagcaaatgcgcctcatatactgccaaaaatagtgttagtttcattaatgtgaattttccagcattcagtagttgtaatgttagaaacaattgctggtcaagttcaacttgttgctattgtttttaatttgcacaggagtagtatcagaaattagtgtcactgcttgtatctagctgaattttaaacaacagaacattagttttttatgttggtgccaccaactgtaaatgacataagttagttattacaaaacacagtaattagactgttgcaaccatctaaaaccttaggcttccagtctgtgctgttagtgttaagatgtaaagtgcaatcctaagctaacattatctgtgcaagcaccatagaaacatttgcatatctgcatagatcttacaactgtactctttacctccttgtgataaagctttgtctacctgcaaacacagtcaaaggctacagctgcaaaccaaagccaactctaaccatggccaagagctcaaggacagaagcagccacatgctttggtcagccttctgtaacttcaattagtacaaaggaaccttttccatgaactacctgctgttttctgatgacctctgggatcttttcatttagccctaaacaaagaaacaaatatgacaaaaaccacaactaaaaaatgttaattcagtcacagagtaatcttctgaggccaaaagtccatctaaatgcaatgaagatttgctttcattaaagacagaggtgaggacaaaatccgcagtggaagttatgatatgctagaaagcaacaaatgtggatcactgaccaaaacgattatgtacttgatgcaaatgcagattgcatattgttatatatatagtactttgtgtttttgttttccctcattcagtcagttattttcagtggtgaatacatgttgttagaagatgtcttgtatggtcttaatctttgttgtgtactatttttttatagtcttaagttataatgaaaaaacaaaaagtaggaaccaaacataaaaggtctagtaaagccaaaaattaatttcatattgattttaaagtgatctagctgagtttttacactgaaagcaaagattatagcaattgtagtccatggtatttattttcagtcaaaccaaagttacatataattctgcctctgcttatacgggatattaacactaacaatacactcccttcaaagacttgcacaggccaaattgttggaatgctggttttcttgacaattccaaaccccaaaactatgataatgagttatgatgtagttgaaaatagcatagtcagatgtttgcttaaaacctagaaacttaacatgttgcttttcatgtgctgtgccaagtcttgataatactttttcccccaaccaagggacctcataacctgattatggttattgctttacaaacagttttgacagaaggtggctgctagagcttaacatacgttcccgttccatgtgatggaaccggttcttgcaaactaagctcatcattgattctttgctgaagtcagcaaatagagttagagagatacccagtcatctatcacaccaaataaaaggacataacggctttcaaaagggttttcccacttacccaaaaggctttctgaaagcttctacctctgcaaaaaaaaaaaaagaaaaaaaaaaaaagaaaaacattagaacaattatggcagattgcatgaaacgtgagaacgtcacagtaactgctacttttcattatgtttgtctttgggtcatgatcaacgaaccggaagtttacaatatggtattaaaagaaagatgggtatggtgaaagatggttttcagtcatctaggatcctactgtaaggattatctgaaaggaaaaatgggtctttcaggtgcatgttcaaaaggctttgagggactggaagtaactgcgagagttgtaccatcagaagggtggcctaagactacaatgctaaagtatgcatacctcagttagaaaacttttgaaaggaagtctcagccacagaatgcatatacctgtagagttttgcatgggttttatatgaatacaattttaaaaaatagctgcttgcacattataccagaaaaacctccaaaactgcaattgctttgaaaatagattttaggttttttggagtttcctgaaatgcttggtctgtattttgataattgtgcatattatgtaaaaatgttggtggacccataaatgaccagactttttctaagaaaaatgttgctttaatgcatttcatgaatttttactcttatatcattgcttgctagtaatagcaaatctgcttttctgcatctgctttgcgtagctattgtaaggctttgaactaatgtatgtatttattgcttgaacttctgtgcataccttataaagcataatgtctgacaatttaaatggctcatgtattcttgcttctatcataagctgattatggggactatgatcttttgtatacagcaaattttaaactgtagcacaaacatctgtttatgtattggtggaatatacctgttttatttatcttttttgaggtaaactaatttttgatacttttcattactgtgtactatgttcatactttgaattctctgacgttagaagtcatggttgagaattgtaacagctgttattcgttctgtattcatggctttcactgctgaataaaataaaggaccaaacctaggatttgaaagaaaactgtctacctctaacaccagggagttatcagattttattttacatagttttagtctacaaagacacaattgcttaaacctagtgggcttaaggcttatattctatgtggttggattcgtggcacagttgtactatttgaaaatcaattaaaattttatgtgaatgttacaagtatttggtagaattaccactaactgggttttctttagataactcagatatggagaaaatgtcatcagcattctgtgtctacagctgcttaacttcataagaatgcatttctttgtgattagggaatcgaagaatagtcagctaggaatagagctacagaagtacacttacataaaccatcctggactttaatgtccctgggcagattcagtcgcaaaatccaatatgatattttgtaaagttttcaagttggacatttacatttttgagaattttgagacttcatcttacacatgccagtattaacacacatttggacaatagctttattaagtctataaagctattgaaaggaacatggcttacccttgttatttcactagttcaggttgcaacgaaaggtttttttgtccatgaacacttggcatatcttacttagcaaaaaagaaggatgtacattttactatagaattaatgtatgaacagtgtgtcactgctgttggatgtaaaaatgtatatgaaaccatttcattcacttgattacatttctgaagtataaataaaaaaatctaattctttttgaccca

SEQ ID NO: 60 - protein, 714 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 61 - RNA, 6543 nt, Homo sapiens
  source 1..6543: mol_type = mRNA
acagacatgaaagctaaccgaggacttgagagactcaaactggtgcttttgtctctctctctctgtctttctctctctcacacacacactcacacactcacacacatgcacacacacacatacacacacacaaaaatgaagcacttactttagaaagattatggtaagcatgctggctcagtcttgaacctttgtcacccctcacgttgcacaccaaagacataccctagtgattaaatgctgattttgtgtacgattgtccacggacgccaaaacaatcacagagctgcttgatttgttttaattaccagcacaaaatgccatcagtctgggacgtgatcgggcagaggtgtactcacagtagtgtaaatactgctgtaaatagttgtctgatggtggctttgacagtgagctagcttctgagttttcccttctttttatactgttttctgtgctggcttttttgaatcttcctaatttttcatctctttaacaaactcctatgaagttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatgagaactgacttgtgaaacctcagcgtgaagggacatatcactgaccttcataaccactccacaaccatgaatatttgacaaatttttactgtgactatttattaagcatggataaaggagacagccctaaaggaacttactaagccagccctttgggattcagtaccaacaggcaaattgcttgttttcttcttcttcttcttctttttttttttttttttagaaaaaaagacaaaaactgattttcttgaaaaaaaaaaatgaactgttctttctataatggctttgcccatttaaaaaatgtggctcttaagggttcatgaaatgactgaatatgaggatacatgtcctgtagaaagcaaatgcgcctcatatactgccaaaaatagtgttagtttcattaatgtgaattttccagcattcagtagttgtaatgttagaaacaattgctggtcaagttcaacttgttgctattgtttttaatttgcacaggagtagtatcagaaattagtgtcactgcttgtatctagctgaattttaaacaacagaacattagttttttatgttggtgccaccaactgtaaatgacataagttagttattacaaaacacagtaattagactgttgcaaccatctaaaaccttaggcttccagtctgtgctgttagtgttaagatgtaaagtgcaatcctaagctaacattatctgtgcaagcaccatagaaacatttgcatatctgcatagatcttacaactgtactctttacctccttgtgataaagctttgtctacctgcaaacacagtcaaaggctacagctgcaaaccaaagccaactctaaccatggccaagagctcaaggacagaagcagccacatgctttggtcagccttctgtaacttcaattagtacaaaggaaccttttccatgaactacctgctgttttctgatgacctctgggatcttttcatttagccctaaacaaagaaacaaatatgacaaaaaccacaactaaaaaatgttaattcagtcacagagtaatcttctgaggccaaaagtccatctaaatgcaatgaagatttgctttcattaaagacagaggtgaggacaaaatccgcagtggaagttatgatatgctagaaagcaacaaatgtggatcactgaccaaaacgattatgtacttgatgcaaatgcagattgcatattgttatatatatagtactttgtgtttttgttttccctcattcagtcagttattttcagtggtgaatacatgttgttagaagatgtcttgtatggtcttaatctttgttgtgtactatttttttatagtcttaagttataatgaaaaaacaaaaagtaggaaccaaacataaaaggtctagtaaagccaaaaattaatttcatattgattttaaagtgatctagctgagtttttacactgaaagcaaagattatagcaattgtagtccatggtatttattttcagtcaaaccaaagttacatataattctgcctctgcttatacgggatattaacactaacaatacactcccttcaaagacttgcacaggccaaattgttggaatgctggttttcttgacaattccaaaccccaaaactatgataatgagttatgatgtagttgaaaatagcatagtcagatgtttgcttaaaacctagaaacttaacatgttgcttttcatgtgctgtgccaagtcttgataatactttttcccccaaccaagggacctcataacctgattatggttattgctttacaaacagttttgacagaaggtggctgctagagcttaacatacgttcccgttccatgtgatggaaccggttcttgcaaactaagctcatcattgattctttgctgaagtcagcaaatagagttagagagatacccagtcatctatcacaccaaataaaaggacataacggctttcaaaagggttttcccacttacccaaaaggctttctgaaagcttctacctctgcaaaaaaaaaaaaagaaaaaaaaaaaaagaaaaacattagaacaattatggcagattgcatgaaacgtgagaacgtcacagtaactgctacttttcattatgtttgtctttgggtcatgatcaacgaaccggaagtttacaatatggtattaaaagaaagatgggtatggtgaaagatggttttcagtcatctaggatcctactgtaaggattatctgaaaggaaaaatgggtctttcaggtgcatgttcaaaaggctttgagggactggaagtaactgcgagagttgtaccatcagaagggtggcctaagactacaatgctaaagtatgcatacctcagttagaaaacttttgaaaggaagtctcagccacagaatgcatatacctgtagagttttgcatgggttttatatgaatacaattttaaaaaatagctgcttgcacattataccagaaaaacctccaaaactgcaattgctttgaaaatagattttaggttttttggagtttcctgaaatgcttggtctgtattttgataattgtgcatattatgtaaaaatgttggtggacccataaatgaccagactttttctaagaaaaatgttgctttaatgcatttcatgaatttttactcttatatcattgcttgctagtaatagcaaatctgcttttctgcatctgctttgcgtagctattgtaaggctttgaactaatgtatgtatttattgcttgaacttctgtgcataccttataaagcataatgtctgacaatttaaatggctcatgtattcttgcttctatcataagctgattatggggactatgatcttttgtatacagcaaattttaaactgtagcacaaacatctgtttatgtattggtggaatatacctgttttatttatcttttttgaggtaaactaatttttgatacttttcattactgtgtactatgttcatactttgaattctctgacgttagaagtcatggttgagaattgtaacagctgttattcgttctgtattcatggctttcactgctgaataaaataaaggaccaaacctaggatttgaaagaaaactgtctacctctaacaccagggagttatcagattttattttacatagttttagtctacaaagacacaattgcttaaacctagtgggcttaaggcttatattctatgtggttggattcgtggcacagttgtactatttgaaaatcaattaaaattttatgtgaatgttacaagtatttggtagaattaccactaactgggttttctttagataactcagatatggagaaaatgtcatcagcattctgtgtctacagctgcttaacttcataagaatgcatttctttgtgattagggaatcgaagaatagtcagctaggaatagagctacagaagtacacttacataaaccatcctggactttaatgtccctgggcagattcagtcgcaaaatccaatatgatattttgtaaagttttcaagttggacatttacatttttgagaattttgagacttcatcttacacatgccagtattaacacacatttggacaatagctttattaagtctataaagctattgaaaggaacatggcttacccttgttatttcactagttcaggttgcaacgaaaggtttttttgtccatgaacacttggcatatcttacttagcaaaaaagaaggatgtacattttactatagaattaatgtatgaacagtgtgtcactgctgttggatgtaaaaatgtatatgaaaccatttcattcacttgattacatttctgaagtataaataaaaaaatctaattctttttgaccca

SEQ ID NO: 62 - protein, 715 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 63 - RNA, 1410 nt, Homo sapiens
  source 1..1410: mol_type = mRNA
gtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctgtaagtgcatattgctttataaacagtaaatagctctaccaatgtaacagactaagaaaatgaacaatttagtgacagttagaaaacaatgagtgtgatgaaaaatacggcaataaaatgaaagtaaaatgtaa

SEQ ID NO: 64 - protein, 432 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPVSAYCFINSK

SEQ ID NO: 65 - RNA, 1485 nt, Homo sapiens
  source 1..1485: mol_type = mRNA
gtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggaattgcttccagaaacaaaattatgtatctgtggccactcttctggtgatgggcatcctcacaacacatttgcagtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcagcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctgtaagtgcatattgctttataaacagtaaatagctctaccaatgtaacagactaagaaaatgaacaatttagtgacagttagaaaacaatgagtgtgatgaaaaatacggcaataaaatgaaagtaaaatgtaa

SEQ ID NO: 66 - protein, 457 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQELLPETKLCICGHSSGDGHPHNTFAVPVSVAMMTPQVITPQQMQQILQQQVLSPQQLQALLQQQQAVMLQQQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPVSAYCFINSK

SEQ ID NO: 67 - RNA, 6594 nt, Homo sapiens
  source 1..6594: mol_type = mRNA
acagacatgaaagctaaccgaggacttgagagactcaaactggtgcttttgtctctctctctctgtctttctctctctcacacacacactcacacactcacacacatgcacacacacacatacacacacacaaaaatgaagcacttactttagaaagattatggtaagcatgctggctcagtcttgaacctttgtcacccctcacgttgcacaccaaagacataccctagtgattaaatgctgattttgtgtacgattgtccacggacgccaaaacaatcacagagctgcttgatttgttttaattaccagcacaaaatgccatcagtctgggacgtgatcgggcagaggtgtactcacagtagtgtaaatactgctgtaaatagttgtctgatggtggctttgacagtgagctagcttctgagttttcccttctttttatactgttttctgtgctggcttttttgaatcttcctaatttttcatctctttaacaaactcctatgaagttgaaaccgggaagtttgctctaacatttccagagaaggtattaagtcatgatgcaggaatctgcgacagagacaataagcaacagttcaatgaatcaaaatggaatgagcactctaagcagccaattagatgctggcagcagagatggaagatcaagtggtgacaccagctctgaagtaagcacagtagaactgctgcatctgcaacaacagcaggctctccaggcagcaagacaacttcttttacagcagcaaacaagtggattgaaatctcctaagagcagtgataaacagagaccactgcaggtgcctgtgtcagtggccatgatgactccccaggtgatcacccctcagcaaatgcagcagatccttcagcaacaagtcctgtctcctcagcagctacaagcccttctccaacaacagcaggctgtcatgctgcagcaggattttttggattctggattggaaaatttcagagctgccttggaaaaaaatcaacaactacaagagttttacaagaaacagcaagagcagttacatcttcagcttttgcagcagcagcagcaacagcagcagcagcaacaacagcagcaacaacagcagcagcaacaacaacaacaacagcagcaacaacagcagcagcagcagcaacagcagcagcagcagcaacagcatcctggaaagcaagcgaaagagcagcagcagcagcagcagcagcaacagcaattggcagcccagcagcttgtcttccagcagcagcttctccagatgcaacaactccagcagcagcagcatctgctcagccttcagcgtcagggactcatctccattccacctggccaggcagcacttcctgtccaatcgctgcctcaagctggcttaagtcctgctgagattcagcagttatggaaagaagtgactggagttcacagtatggaagacaatggcattaaacatggagggctagacctcactactaacaattcctcctcgactacctcctccaacacttccaaagcatcaccaccaataactcatcattccatagtgaatggacagtcttcagttctaagtgcaagacgagacagctcgtcacatgaggagactggggcctctcacactctctatggccatggagtttgcaaatggccaggctgtgaaagcatttgtgaagattttggacagtttttaaagcaccttaacaatgaacacgcattggatgaccgaagcactgctcagtgtcgagtgcaaatgcaggtggtgcaacagttagaaatacagctttctaaagaacgcgaacgtcttcaagcaatgatgacccacttgcacatgcgaccctcagagcccaaaccatctcccaaacctctaaatctggtgtctagtgtcaccatgtcgaagaatatgttggagacatccccacagagcttacctcaaacccctaccacaccaacggccccagtcaccccgattacccagggaccctcagtaatcaccccagccagtgtgcccaatgtgggagccatacgaaggcgacattcagacaaatacaacattcccatgtcatcagaaattgccccaaactatgaattttataaaaatgcagatgtcagacctccatttacttatgcaactctcataaggcaggctatcatggagtcatctgacaggcagttaacacttaatgaaatttacagctggtttacacggacatttgcttacttcaggcgtaatgcagcaacttggaagaatgcagtacgtcataatcttagcctgcacaagtgttttgttcgagtagaaaatgttaaaggagcagtatggactgtggatgaagtagaataccagaagcgaaggtcacaaaagataacaggaagtccaaccttagtaaaaaatatacctaccagtttaggctatggagcagctcttaatgccagtttgcaggctgccttggcagagagcagtttacctttgctaagtaatcctggactgataaataatgcatccagtggcctactgcaggccgtccacgaagacctcaatggttctctggatcacattgacagcaatggaaacagtagtccgggctgctcacctcagccgcacatacattcaatccacgtcaaggaagagccagtgattgcagaggatgaagactgcccaatgtccttagtgacaacagctaatcacagtccagaattagaagacgacagagagattgaagaagagcctttatctgaagatctggaatgagaactgacttgtgaaacctcagcgtgaagggacatatcactgaccttcataaccactccacaaccatgaatatttgacaaatttttactgtgactatttattaagcatggataaaggagacagccctaaaggaacttactaagccagccctttgggattcagtaccaacaggcaaattgcttgttttcttcttcttcttcttctttttttttttttttttagaaaaaaagacaaaaactgattttcttgaaaaaaaaaaatgaactgttctttctataatggctttgcccatttaaaaaatgtggctcttaagggttcatgaaatgactgaatatgaggatacatgtcctgtagaaagcaaatgcgcctcatatactgccaaaaatagtgttagtttcattaatgtgaattttccagcattcagtagttgtaatgttagaaacaattgctggtcaagttcaacttgttgctattgtttttaatttgcacaggagtagtatcagaaattagtgtcactgcttgtatctagctgaattttaaacaacagaacattagttttttatgttggtgccaccaactgtaaatgacataagttagttattacaaaacacagtaattagactgttgcaaccatctaaaaccttaggcttccagtctgtgctgttagtgttaagatgtaaagtgcaatcctaagctaacattatctgtgcaagcaccatagaaacatttgcatatctgcatagatcttacaactgtactctttacctccttgtgataaagctttgtctacctgcaaacacagtcaaaggctacagctgcaaaccaaagccaactctaaccatggccaagagctcaaggacagaagcagccacatgctttggtcagccttctgtaacttcaattagtacaaaggaaccttttccatgaactacctgctgttttctgatgacctctgggatcttttcatttagccctaaacaaagaaacaaatatgacaaaaaccacaactaaaaaatgttaattcagtcacagagtaatcttctgaggccaaaagtccatctaaatgcaatgaagatttgctttcattaaagacagaggtgaggacaaaatccgcagtggaagttatgatatgctagaaagcaacaaatgtggatcactgaccaaaacgattatgtacttgatgcaaatgcagattgcatattgttatatatatagtactttgtgtttttgttttccctcattcagtcagttattttcagtggtgaatacatgttgttagaagatgtcttgtatggtcttaatctttgttgtgtactatttttttatagtcttaagttataatgaaaaaacaaaaagtaggaaccaaacataaaaggtctagtaaagccaaaaattaatttcatattgattttaaagtgatctagctgagtttttacactgaaagcaaagattatagcaattgtagtccatggtatttattttcagtcaaaccaaagttacatataattctgcctctgcttatacgggatattaacactaacaatacactcccttcaaagacttgcacaggccaaattgttggaatgctggttttcttgacaattccaaaccccaaaactatgataatgagttatgatgtagttgaaaatagcatagtcagatgtttgcttaaaacctagaaacttaacatgttgcttttcatgtgctgtgccaagtcttgataatactttttcccccaaccaagggacctcataacctgattatggttattgctttacaaacagttttgacagaaggtggctgctagagcttaacatacgttcccgttccatgtgatggaaccggttcttgcaaactaagctcatcattgattctttgctgaagtcagcaaatagagttagagagatacccagtcatctatcacaccaaataaaaggacataacggctttcaaaagggttttcccacttacccaaaaggctttctgaaagcttctacctctgcaaaaaaaaaaaaagaaaaaaaaaaaaagaaaaacattagaacaattatggcagattgcatgaaacgtgagaacgtcacagtaactgctacttttcattatgtttgtctttgggtcatgatcaacgaaccggaagtttacaatatggtattaaaagaaagatgggtatggtgaaagatggttttcagtcatctaggatcctactgtaaggattatctgaaaggaaaaatgggtctttcaggtgcatgttcaaaaggctttgagggactggaagtaactgcgagagttgtaccatcagaagggtggcctaagactacaatgctaaagtatgcatacctcagttagaaaacttttgaaaggaagtctcagccacagaatgcatatacctgtagagttttgcatgggttttatatgaatacaattttaaaaaatagctgcttgcacattataccagaaaaacctccaaaactgcaattgctttgaaaatagattttaggttttttggagtttcctgaaatgcttggtctgtattttgataattgtgcatattatgtaaaaatgttggtggacccataaatgaccagactttttctaagaaaaatgttgctttaatgcatttcatgaatttttactcttatatcattgcttgctagtaatagcaaatctgcttttctgcatctgctttgcgtagctattgtaaggctttgaactaatgtatgtatttattgcttgaacttctgtgcataccttataaagcataatgtctgacaatttaaatggctcatgtattcttgcttctatcataagctgattatggggactatgatcttttgtatacagcaaattttaaactgtagcacaaacatctgtttatgtattggtggaatatacctgttttatttatcttttttgaggtaaactaatttttgatacttttcattactgtgtactatgttcatactttgaattctctgacgttagaagtcatggttgagaattgtaacagctgttattcgttctgtattcatggctttcactgctgaataaaataaaggaccaaacctaggatttgaaagaaaactgtctacctctaacaccagggagttatcagattttattttacatagttttagtctacaaagacacaattgcttaaacctagtgggcttaaggcttatattctatgtggttggattcgtggcacagttgtactatttgaaaatcaattaaaattttatgtgaatgttacaagtatttggtagaattaccactaactgggttttctttagataactcagatatggagaaaatgtcatcagcattctgtgtctacagctgcttaacttcataagaatgcatttctttgtgattagggaatcgaagaatagtcagctaggaatagagctacagaagtacacttacataaaccatcctggactttaatgtccctgggcagattcagtcgcaaaatccaatatgatattttgtaaagttttcaagttggacatttacatttttgagaattttgagacttcatcttacacatgccagtattaacacacatttggacaatagctttattaagtctataaagctattgaaaggaacatggcttacccttgttatttcactagttcaggttgcaacgaaaggtttttttgtccatgaacacttggcatatcttacttagcaaaaaagaaggatgtacattttactatagaattaatgtatgaacagtgtgtcactgctgttggatgtaaaaatgtatatgaaaccatttcattcacttgattacatttctgaagtataaataaaaaaatctaattctttttgaccca

SEQ ID NO: 68 - protein, 732 aa, Homo sapiens
MMQESATETISNSSMNQNGMSTLSSQLDAGSRDGRSSGDTSSEVSTVELLHLQQQQALQAARQLLLQQQTSGLKSPKSSDKQRPLQVPVSVAMMTPQVITPQQMQQILQQQVLSPQQLQALLQQQQAVMLQQDFLDSGLENFRAALEKNQQLQEFYKKQQEQLHLQLLQQQQQQQQQQQQQQQQQQQQQQQQQQQQQQQQQQQQQQQQHPGKQAKEQQQQQQQQQQLAAQQLVFQQQLLQMQQLQQQQHLLSLQRQGLISIPPGQAALPVQSLPQAGLSPAEIQQLWKEVTGVHSMEDNGIKHGGLDLTTNNSSSTTSSNTSKASPPITHHSIVNGQSSVLSARRDSSSHEETGASHTLYGHGVCKWPGCESICEDFGQFLKHLNNEHALDDRSTAQCRVQMQVVQQLEIQLSKERERLQAMMTHLHMRPSEPKPSPKPLNLVSSVTMSKNMLETSPQSLPQTPTTPTAPVTPITQGPSVITPASVPNVGAIRRRHSDKYNIPMSSEIAPNYEFYKNADVRPPFTYATLIRQAIMESSDRQLTLNEIYSWFTRTFAYFRRNAATWKNAVRHNLSLHKCFVRVENVKGAVWTVDEVEYQKRRSQKITGSPTLVKNIPTSLGYGAALNASLQAALAESSLPLLSNPGLINNASSGLLQAVHEDLNGSLDHIDSNGNSSPGCSPQPHIHSIHVKEEPVIAEDEDCPMSLVTTANHSPELEDDREIEEEPLSEDLE

SEQ ID NO: 69 - RNA, 5862 nt, Homo sapiens
  source 1..5862: mol_type = mRNA
actcgcagcccaggctcagtcgcagcccctcggagtccggagcccgccgtccctgcggaccggacagtgcggccggcggccggcggccgggacggagcccccagcccgcgaggagggcgcggcgcaggcggcggcggcgcggcggggaggagcccgggccggaaccagggctgggccgggggcggggacgccggggtccgggagcccgggagccggagcgagctgacgagcgtcgcggaaggaccgggaaggaagagcggagccggagcgagccaagcagcccacaaagtgccatgccccggcggggttgaggcgcgcggcggtcggcccggcgcgctgggaggacgcccgggagctgcgcgacgcggggcggcgcggaagggcagccccggcgggcggcggcgggcccgggctgcgaccggagcgagccccatgccccgcgcgggctgacgggccggagcccgcacggagcggccgggcgggacaggtaccgctagagcgacatgatggtggaatctgcctcggagacaatcaggtcggctccatctggtcagaatggcgtgggcagcctctctgggcaagccgatggcagcagcggcggggccacagggacaactgcaagtggcacgggcagggaagtgaccacgggtgcagacagcaatggtgagatgagtcccgcagagctgctgcacttccagcagcaacaggtgcctgtgtcggtggccatgatgtcgccgcagatgcttaccccgcaacagatgcagcagatcctgtcgcccccgcagctgcaggccttgctccagcagcagcaagccctcatgctccagcagctacaggagtactacaagaagcagcaggagcagctccacctgcagctcctcacccagcagcaggctgggaaaccgcagcccaaagaggcactggggaacaagcagctggccttccagcagcagctcctgcaaatgcaacagttgcagcagcagcacctgctcaacctgcagaggcaggggctggtcagcctgcagcccaaccaagcctcggggcccctccagacccttccgcaagcagctgtttgcccaacagacctgccccagctgtggaagggcgagggtgcccccgggcagcctgccgaggacagcgtcaagcaggaggggctggacctcactggcacggccgccaccgctacctcgtttgccgctccccccaaggtctcaccccccctctcccaccataccctgcccaacggacagcctactgtgctcacatctcggagagacagctcttcccacgaggagacccccggctcccaccccctgtacggacacggagagtgcaagtggccaggctgtgagaccctgtgtgaagacctgggccagtttatcaaacacctcaacacagagcacgccctggatgaccggagtacagcccagtgccgggtacagatgcaggtggtgcagcagctggagatccagctcgccaaggagagcgagcggctgcaggccatgatggcccacctgcacatgcggccctcggagcccaagcccttcagccagccagtgaccgtctctgcagcagactcattcccagatggtctcgtgcaccccccgacctcggccgcagcccctgtcacccctctacggccccctggcctgggctctgcctccctgcatggtgggggcccagcccgtcggagaagcagtgacaagttctgctcccccatctcctcagagctggcccagaatcatgagttctacaagaacgccgacgtccggccccccttcacctacgcctccctcatccgccaggccatcctggaaacccctgacaggcagctgaccctgaatgagatctataactggttcaccaggatgttcgcctatttccgcagaaacactgccacctggaagaacgccgtgcgccacaacctcagcctgcacaagtgcttcgtccgcgtggagaacgtcaagggtgccgtgtggactgtggacgagcgggagtatcagaagcggagaccgccaaagatgacagggagccccaccctggtgaagaacatgatctctggcctcagctatggagcacttaatgccagctaccaggccgccctggccgagagcagcttccccctcctcaacagccctggcatgctgaaccctggctccgccagcagcctgctgcccctcagccacgatgacgtgggtgcccccgtggagccgctgcccagcaacggcagcagcagccctcctcgcctctccccgccccagtacagccaccaggtgcaggtgaaggaggagccagcagaggcagaggaagacaggcagcccgggcctcccctgggcgcccctaaccccagcgcctcggggcctccggaagacagggacctggaggaggagctgccgggagaagaactgtcctaagggcctgtagtgaccggcagggctggggtgagacccctcccttccagaatccaggccccatctcccccaactccacagcccctcccgagcctcaaggcaagtccaggactcagaccggggaggcccgggccagcagctcccagtgtgacctgacaaaaacacgtaggggcagggacggtccccacccccagggacacaacccctggtcttggaccagtagaggacacggagggttcagacccctcctcagaccctccccacatctgaaactgcctccccccaaccaccagcagcagcagggccctcctcccccaccagctctccccacagggcccctcagcatcatggagacccgcaggcggggcttagccacccctcaaacccagggccccctggcacctggctctggccgtgttttctggccagaggcccccactttcctaactcgtgctcccttccgccttcttttccgtactgtgaagaaagaactctccaccccagctcccaccctgccctggcctgggtggaggaactgtgcctccatccccagaagaaacagccccctctgctgctggggtgggactgtctgtgtgccctgtgggggtccgtgtgagcaggcccacctggctccagacccgcccccaacctgagacagaaccaggctgagccaggcctccacccccacccccgtttgctgggggctcctccagccgcccccatgggaagaggcctggtaccgcctcacccacagaggtctgtgccaggtgcgcttctgcaggtggagccaagctctccctgaggccagaggcggggcctgggccgggagcccaggggaaggccaggctggaccccggctccacacccacatccagcctgcaggcctctctgcagtcctctcaccctccctcagctccccttcctctgcagtcaccctcagctccccttccttgcccgcctctccccccgccgccccaccagttaaacggatgaccaaagacctttcttatgccggaagcaaaaaccaaaactttttgttggctttttcctttgtcgcctccccagcacctgccctcccagtctcccaccccggccccaggctggaagccctccctccacttaagttattgttttaaaccaaagtttacagtgtctgttggtggccaagaccttctctctccacccctcctccatccaccctgaggaccctggggctcagtggaggcagggccctgcccccctcccttccgctcctgcccagcctgggggaaggagaaaggaggggagaaagcgggctctcaccccctcaggagtgggcacgggagcccttctccctgaccctgggctgcttcctgggggctctccagacccctctctaggaccaagtcacccgtcgtgctgggagtgtggattctagcaaaagagctggaaaaaagtcagactctccacagaccccctatgggggacccccaactcaaggccaaggactgggcgtatcggatgctcataacacccctggcctggcccctttactgagaagactccttggatatttcccaagaaccccccacatacacccctcacaagccacccctcctgagaggcagggggccctccgccccctccccatgtattccccacctgtgttccgtttgaccagcacagaaatattaaacgtcctctattcaccgggccctgtgtgtgtcaccgaggtgcgggaggggaggagcattaaagctgaaagatcgctctgctcggggagcctgggcagagcagcagcaacgtgagggtcgctgtggtggtggtttctgtgagtggatggaatgagcagccctgcaggggcgctgggcatgtgccctcactgtggacagggcccacccacctccggttcccctgtgcctcctgtcccttccacgcttaaacagggttctctgtcattttcctgttttcttccagagtcccaatcctttgccctagttctttcactagtttgaaatccaagttcttgccagagtgttggagcaaggcagctgatttgctgcagggatggagaggaccaccgccgcagggttcttttacctgtgccaccagctctggagacatccacacccattcccaggtgtccttccaggcccattgtccacgtcttcaagggggctgcctggataggcgtgtgtgtgtgtagtgcccaggtgtggtggtggcatcctgaagcagtaggaccattagtgtgcgtgcacacccacgtggcacactgtgtggtgaccatggtcatcatagtgggctcacggcggaaacgggatcattcaacctatacaaaggggaccctggataggctgcaaggaaagaggccaagggccaagctgctaagccaaagatggcccctgacacctcccccagccccagcacaccagcctgcacctcttatcccgtgttaagtcctggttccccacctgctgcccctcctcaacacagagcccctcccgccgcctcagacccctgtgcacatccccagggcctcagccgtctcattggtctttattttttattttttttaagatggagtttcgctcttattgcccagactggagtgcagtgatgctatctcggctcactgcaacctttgcctcccaggttcaagtgattctcctgcctcagcctcccaagtagctgggattacaggcgtgcaccaccacgccgggctaaatttttttgtatttttagtagagacggggtttctctatgttggtcaggctgatctcgaactcccgacctcaggtgatccgccagcctcagcctcccaaagtgctgggattacaggcgtgagccactgcacccggctctcactggtcttacgccaccttctggacactccctccttgagggcagaaaggagtcccaggcctgtccctagggacaaggcccagggaagagtgtatttggggagcaggggaggggagggtgttgagaaagctgaactggagtcaatcacccttcccacaaatcaccaaactgctggaactctccagccaaatgctgggagaaggacctggagggtgagtctttgctgacctctctctactctcaggcatgtcttttgtccttttcgtccatctatttctgtctgtcgctcactcgccccgctttctctgtctcaccttcatccactctgcaggcctgctccaccacagccctaatcctctggacgcttgtgtagggcctggggtgaattccctgtcccccatggtacctcgagaggggctggggagctcagcttggtctcagagtctccccaccagatactgtttaaaaaagtagcactgatgtgttttgtaatctgcccctcccagccctccgtggaggctgccagggccttgtacggtaaacctagctgcatgtaatctgtggacaatggcattctctacaatgcaataaaaacaattacccatga

SEQ ID NO: 70 - protein, 636 aa, Homo sapiens
MMVESASETIRSAPSGQNGVGSLSGQADGSSGGATGTTASGTGREVTTGADSNGEMSPAELLHFQQQQVPVSVAMMSPQMLTPQQMQQILSPPQLQALLQQQQALMLQQLQEYYKKQQEQLHLQLLTQQQAGKPQPKEALGNKQLAFQQQLLQMQQLQQQHLLNLQRQGLVSLQPNQASGPLQTLPQAAVCPTDLPQLWKGEGAPGQPAEDSVKQEGLDLTGTAATATSFAAPPKVSPPLSHHTLPNGQPTVLTSRRDSSSHEETPGSHPLYGHGECKWPGCETLCEDLGQFIKHLNTEHALDDRSTAQCRVQMQVVQQLEIQLAKESERLQAMMAHLHMRPSEPKPFSQPVTVSAADSFPDGLVHPPTSAAAPVTPLRPPGLGSASLHGGGPARRRSSDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLNSPGMLNPGSASSLLPLSHDDVGAPVEPLPSNGSSSPPRLSPPQYSHQVQVKEEPAEAEEDRQPGPPLGAPNPSASGPPEDRDLEEELPGEELS

SEQ ID NO: 71 - RNA, 5898 nt, Homo sapiens
  source 1..5898: mol_type = mRNA
actcgcagcccaggctcagtcgcagcccctcggagtccggagcccgccgtccctgcggaccggacagtgcggccggcggccggcggccgggacggagcccccagcccgcgaggagggcgcggcgcaggcggcggcggcgcggcggggaggagcccgggccggaaccagggctgggccgggggcggggacgccggggtccgggagcccgggagccggagcgagctgacgagcgtcgcggaaggaccgggaaggaagagcggagccggagcgagccaagcagcccacaaagtgccatgccccggcggggttgaggcgcgcggcggtcggcccggcgcgctgggaggacgcccgggagctgcgcgacgcggggcggcgcggaagggcagccccggcgggcggcggcgggcccgggctgcgaccggagcgagccccatgccccgcgcgggctgacgggccggagcccgcacggagcggccgggcgggacaggtaccgctagagcgacatgatggtggaatctgcctcggagacaatcaggtcggctccatctggtcagaatggcgtgggcagcctctctgggcaagccgatggcagcagcggcggggccacagggacaactgcaagtggcacgggcagggaagtgaccacgggtgcagacagcaatggtgagatgagtcccgcagagctgctgcacttccagcagcaacaggtgcctgtgtcggtggccatgatgtcgccgcagatgcttaccccgcaacagatgcagcagatcctgtcgcccccgcagctgcaggccttgctccagcagcagcaagccctcatgctccagcagctacaggagtactacaagaagcagcaggagcagctccacctgcagctcctcacccagcagcaggctgggaaaccgcagcccaaagaggcactggggaacaagcagctggccttccagcagcagctcctgcaaatgcaacagttgcagcagcagcacctgctcaacctgcagaggcaggggctggtcagcctgcagcccaaccaagcctcggggcccctccagacccttccgcaagcagctgtttgcccaacagacctgccccagctgtggaagggcgagggtgcccccgggcagcctgccgaggacagcgtcaagcaggaggggctggacctcactggcacggccgccaccgctacctcgtttgccgctccccccaaggtctcaccccccctctcccaccataccctgcccaacggacagcctactgtgctcacatctcggagagacagctcttcccacgaggagacccccggctcccaccccctgtacggacacggagagtgcaagtggccaggctgtgagaccctgtgtgaagacctgggccagtttatcaaacacctcaacacagagcacgccctggatgaccggagtacagcccagtgccgggtacagatgcaggtggtgcagcagctggagatccagctcgccaaggagagcgagcggctgcaggccatgatggcccacctgcacatgcggccctcggagcccaagcccttcagccagccactgaacccggtccccggctcctcctcattctccaaggtgaccgtctctgcagcagactcattcccagatggtctcgtgcaccccccgacctcggccgcagcccctgtcacccctctacggccccctggcctgggctctgcctccctgcatggtgggggcccagcccgtcggagaagcagtgacaagttctgctcccccatctcctcagagctggcccagaatcatgagttctacaagaacgccgacgtccggccccccttcacctacgcctccctcatccgccaggccatcctggaaacccctgacaggcagctgaccctgaatgagatctataactggttcaccaggatgttcgcctatttccgcagaaacactgccacctggaagaacgccgtgcgccacaacctcagcctgcacaagtgcttcgtccgcgtggagaacgtcaagggtgccgtgtggactgtggacgagcgggagtatcagaagcggagaccgccaaagatgacagggagccccaccctggtgaagaacatgatctctggcctcagctatggagcacttaatgccagctaccaggccgccctggccgagagcagcttccccctcctcaacagccctggcatgctgaaccctggctccgccagcagcctgctgcccctcagccacgatgacgtgggtgcccccgtggagccgctgcccagcaacggcagcagcagccctcctcgcctctccccgccccagtacagccaccaggtgcaggtgaaggaggagccagcagaggcagaggaagacaggcagcccgggcctcccctgggcgcccctaaccccagcgcctcggggcctccggaagacagggacctggaggaggagctgccgggagaagaactgtcctaagggcctgtagtgaccggcagggctggggtgagacccctcccttccagaatccaggccccatctcccccaactccacagcccctcccgagcctcaaggcaagtccaggactcagaccggggaggcccgggccagcagctcccagtgtgacctgacaaaaacacgtaggggcagggacggtccccacccccagggacacaacccctggtcttggaccagtagaggacacggagggttcagacccctcctcagaccctccccacatctgaaactgcctccccccaaccaccagcagcagcagggccctcctcccccaccagctctccccacagggcccctcagcatcatggagacccgcaggcggggcttagccacccctcaaacccagggccccctggcacctggctctggccgtgttttctggccagaggcccccactttcctaactcgtgctcccttccgccttcttttccgtactgtgaagaaagaactctccaccccagctcccaccctgccctggcctgggtggaggaactgtgcctccatccccagaagaaacagccccctctgctgctggggtgggactgtctgtgtgccctgtgggggtccgtgtgagcaggcccacctggctccagacccgcccccaacctgagacagaaccaggctgagccaggcctccacccccacccccgtttgctgggggctcctccagccgcccccatgggaagaggcctggtaccgcctcacccacagaggtctgtgccaggtgcgcttctgcaggtggagccaagctctccctgaggccagaggcggggcctgggccgggagcccaggggaaggccaggctggaccccggctccacacccacatccagcctgcaggcctctctgcagtcctctcaccctccctcagctccccttcctctgcagtcaccctcagctccccttccttgcccgcctctccccccgccgccccaccagttaaacggatgaccaaagacctttcttatgccggaagcaaaaaccaaaactttttgttggctttttcctttgtcgcctccccagcacctgccctcccagtctcccaccccggccccaggctggaagccctccctccacttaagttattgttttaaaccaaagtttacagtgtctgttggtggccaagaccttctctctccacccctcctccatccaccctgaggaccctggggctcagtggaggcagggccctgcccccctcccttccgctcctgcccagcctgggggaaggagaaaggaggggagaaagcgggctctcaccccctcaggagtgggcacgggagcccttctccctgaccctgggctgcttcctgggggctctccagacccctctctaggaccaagtcacccgtcgtgctgggagtgtggattctagcaaaagagctggaaaaaagtcagactctccacagaccccctatgggggacccccaactcaaggccaaggactgggcgtatcggatgctcataacacccctggcctggcccctttactgagaagactccttggatatttcccaagaaccccccacatacacccctcacaagccacccctcctgagaggcagggggccctccgccccctccccatgtattccccacctgtgttccgtttgaccagcacagaaatattaaacgtcctctattcaccgggccctgtgtgtgtcaccgaggtgcgggaggggaggagcattaaagctgaaagatcgctctgctcggggagcctgggcagagcagcagcaacgtgagggtcgctgtggtggtggtttctgtgagtggatggaatgagcagccctgcaggggcgctgggcatgtgccctcactgtggacagggcccacccacctccggttcccctgtgcctcctgtcccttccacgcttaaacagggttctctgtcattttcctgttttcttccagagtcccaatcctttgccctagttctttcactagtttgaaatccaagttcttgccagagtgttggagcaaggcagctgatttgctgcagggatggagaggaccaccgccgcagggttcttttacctgtgccaccagctctggagacatccacacccattcccaggtgtccttccaggcccattgtccacgtcttcaagggggctgcctggataggcgtgtgtgtgtgtagtgcccaggtgtggtggtggcatcctgaagcagtaggaccattagtgtgcgtgcacacccacgtggcacactgtgtggtgaccatggtcatcatagtgggctcacggcggaaacgggatcattcaacctatacaaaggggaccctggataggctgcaaggaaagaggccaagggccaagctgctaagccaaagatggcccctgacacctcccccagccccagcacaccagcctgcacctcttatcccgtgttaagtcctggttccccacctgctgcccctcctcaacacagagcccctcccgccgcctcagacccctgtgcacatccccagggcctcagccgtctcattggtctttattttttattttttttaagatggagtttcgctcttattgcccagactggagtgcagtgatgctatctcggctcactgcaacctttgcctcccaggttcaagtgattctcctgcctcagcctcccaagtagctgggattacaggcgtgcaccaccacgccgggctaaatttttttgtatttttagtagagacggggtttctctatgttggtcaggctgatctcgaactcccgacctcaggtgatccgccagcctcagcctcccaaagtgctgggattacaggcgtgagccactgcacccggctctcactggtcttacgccaccttctggacactccctccttgagggcagaaaggagtcccaggcctgtccctagggacaaggcccagggaagagtgtatttggggagcaggggaggggagggtgttgagaaagctgaactggagtcaatcacccttcccacaaatcaccaaactgctggaactctccagccaaatgctgggagaaggacctggagggtgagtctttgctgacctctctctactctcaggcatgtcttttgtccttttcgtccatctatttctgtctgtcgctcactcgccccgctttctctgtctcaccttcatccactctgcaggcctgctccaccacagccctaatcctctggacgcttgtgtagggcctggggtgaattccctgtcccccatggtacctcgagaggggctggggagctcagcttggtctcagagtctccccaccagatactgtttaaaaaagtagcactgatgtgttttgtaatctgcccctcccagccctccgtggaggctgccagggccttgtacggtaaacctagctgcatgtaatctgtggacaatggcattctctacaatgcaataaaaacaattacccatga

SEQ ID NO: 72 - protein, 648 aa, Homo sapiens
MMVESASETIRSAPSGQNGVGSLSGQADGSSGGATGTTASGTGREVTTGADSNGEMSPAELLHFQQQQVPVSVAMMSPQMLTPQQMQQILSPPQLQALLQQQQALMLQQLQEYYKKQQEQLHLQLLTQQQAGKPQPKEALGNKQLAFQQQLLQMQQLQQQHLLNLQRQGLVSLQPNQASGPLQTLPQAAVCPTDLPQLWKGEGAPGQPAEDSVKQEGLDLTGTAATATSFAAPPKVSPPLSHHTLPNGQPTVLTSRRDSSSHEETPGSHPLYGHGECKWPGCETLCEDLGQFIKHLNTEHALDDRSTAQCRVQMQVVQQLEIQLAKESERLQAMMAHLHMRPSEPKPFSQPLNPVPGSSSFSKVTVSAADSFPDGLVHPPTSAAAPVTPLRPPGLGSASLHGGGPARRRSSDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLNSPGMLNPGSASSLLPLSHDDVGAPVEPLPSNGSSSPPRLSPPQYSHQVQVKEEPAEAEEDRQPGPPLGAPNPSASGPPEDRDLEEELPGEELS

SEQ ID NO: 73 - RNA, 5988 nt, Homo sapiens
  source 1..5988: mol_type = mRNA
actcgcagcccaggctcagtcgcagcccctcggagtccggagcccgccgtccctgcggaccggacagtgcggccggcggccggcggccgggacggagcccccagcccgcgaggagggcgcggcgcaggcggcggcggcgcggcggggaggagcccgggccggaaccagggctgggccgggggcggggacgccggggtccgggagcccgggagccggagcgagctgacgagcgtcgcggaaggaccgggaaggaagagcggagccggagcgagccaagcagcccacaaagtgccatgccccggcggggttgaggcgcgcggcggtcggcccggcgcgctgggaggacgcccgggagctgcgcgacgcggggcggcgcggaagggcagccccggcgggcggcggcgggcccgggctgcgaccggagcgagccccatgccccgcgcgggctgacgggccggagcccgcacggagcggccgggcgggacaggtaccgctagagcgacatgatggtggaatctgcctcggagacaatcaggtcggctccatctggtcagaatggcgtgggcagcctctctgggcaagccgatggcagcagcggcggggccacagggacaactgcaagtggcacgggcagggaagtgaccacgggtgcagacagcaatggtgagatgagtcccgcagagctgctgcacttccagcagcaacaggctctccaagtggcccggcagttcctgctgcagcaggcctcaggcctgagctccccagggaacaatgacagcaaacagtctgcctctgctgtgcaggtgcctgtgtcggtggccatgatgtcgccgcagatgcttaccccgcaacagatgcagcagatcctgtcgcccccgcagctgcaggccttgctccagcagcagcaagccctcatgctccagcaggagtactacaagaagcagcaggagcagctccacctgcagctcctcacccagcagcaggctgggaaaccgcagcccaaagaggcactggggaacaagcagctggccttccagcagcagctcctgcaaatgcaacagttgcagcagcagcacctgctcaacctgcagaggcaggggctggtcagcctgcagcccaaccaagcctcggggcccctccagacccttccgcaagcagctgtttgcccaacagacctgccccagctgtggaagggcgagggtgcccccgggcagcctgccgaggacagcgtcaagcaggaggggctggacctcactggcacggccgccaccgctacctcgtttgccgctccccccaaggtctcaccccccctctcccaccataccctgcccaacggacagcctactgtgctcacatctcggagagacagctcttcccacgaggagacccccggctcccaccccctgtacggacacggagagtgcaagtggccaggctgtgagaccctgtgtgaagacctgggccagtttatcaaacacctcaacacagagcacgccctggatgaccggagtacagcccagtgccgggtacagatgcaggtggtgcagcagctggagatccagctcgccaaggagagcgagcggctgcaggccatgatggcccacctgcacatgcggccctcggagcccaagcccttcagccagccactgaacccggtccccggctcctcctcattctccaaggtgaccgtctctgcagcagactcattcccagatggtctcgtgcaccccccgacctcggccgcagcccctgtcacccctctacggccccctggcctgggctctgcctccctgcatggtgggggcccagcccgtcggagaagcagtgacaagttctgctcccccatctcctcagagctggcccagaatcatgagttctacaagaacgccgacgtccggccccccttcacctacgcctccctcatccgccaggccatcctggaaacccctgacaggcagctgaccctgaatgagatctataactggttcaccaggatgttcgcctatttccgcagaaacactgccacctggaagaacgccgtgcgccacaacctcagcctgcacaagtgcttcgtccgcgtggagaacgtcaagggtgccgtgtggactgtggacgagcgggagtatcagaagcggagaccgccaaagatgacagggagccccaccctggtgaagaacatgatctctggcctcagctatggagcacttaatgccagctaccaggccgccctggccgagagcagcttccccctcctcaacagccctggcatgctgaaccctggctccgccagcagcctgctgcccctcagccacgatgacgtgggtgcccccgtggagccgctgcccagcaacggcagcagcagccctcctcgcctctccccgccccagtacagccaccaggtgcaggtgaaggaggagccagcagaggcagaggaagacaggcagcccgggcctcccctgggcgcccctaaccccagcgcctcggggcctccggaagacagggacctggaggaggagctgccgggagaagaactgtcctaagggcctgtagtgaccggcagggctggggtgagacccctcccttccagaatccaggccccatctcccccaactccacagcccctcccgagcctcaaggcaagtccaggactcagaccggggaggcccgggccagcagctcccagtgtgacctgacaaaaacacgtaggggcagggacggtccccacccccagggacacaacccctggtcttggaccagtagaggacacggagggttcagacccctcctcagaccctccccacatctgaaactgcctccccccaaccaccagcagcagcagggccctcctcccccaccagctctccccacagggcccctcagcatcatggagacccgcaggcggggcttagccacccctcaaacccagggccccctggcacctggctctggccgtgttttctggccagaggcccccactttcctaactcgtgctcccttccgccttcttttccgtactgtgaagaaagaactctccaccccagctcccaccctgccctggcctgggtggaggaactgtgcctccatccccagaagaaacagccccctctgctgctggggtgggactgtctgtgtgccctgtgggggtccgtgtgagcaggcccacctggctccagacccgcccccaacctgagacagaaccaggctgagccaggcctccacccccacccccgtttgctgggggctcctccagccgcccccatgggaagaggcctggtaccgcctcacccacagaggtctgtgccaggtgcgcttctgcaggtggagccaagctctccctgaggccagaggcggggcctgggccgggagcccaggggaaggccaggctggaccccggctccacacccacatccagcctgcaggcctctctgcagtcctctcaccctccctcagctccccttcctctgcagtcaccctcagctccccttccttgcccgcctctccccccgccgccccaccagttaaacggatgaccaaagacctttcttatgccggaagcaaaaaccaaaactttttgttggctttttcctttgtcgcctccccagcacctgccctcccagtctcccaccccggccccaggctggaagccctccctccacttaagttattgttttaaaccaaagtttacagtgtctgttggtggccaagaccttctctctccacccctcctccatccaccctgaggaccctggggctcagtggaggcagggccctgcccccctcccttccgctcctgcccagcctgggggaaggagaaaggaggggagaaagcgggctctcaccccctcaggagtgggcacgggagcccttctccctgaccctgggctgcttcctgggggctctccagacccctctctaggaccaagtcacccgtcgtgctgggagtgtggattctagcaaaagagctggaaaaaagtcagactctccacagaccccctatgggggacccccaactcaaggccaaggactgggcgtatcggatgctcataacacccctggcctggcccctttactgagaagactccttggatatttcccaagaaccccccacatacacccctcacaagccacccctcctgagaggcagggggccctccgccccctccccatgtattccccacctgtgttccgtttgaccagcacagaaatattaaacgtcctctattcaccgggccctgtgtgtgtcaccgaggtgcgggaggggaggagcattaaagctgaaagatcgctctgctcggggagcctgggcagagcagcagcaacgtgagggtcgctgtggtggtggtttctgtgagtggatggaatgagcagccctgcaggggcgctgggcatgtgccctcactgtggacagggcccacccacctccggttcccctgtgcctcctgtcccttccacgcttaaacagggttctctgtcattttcctgttttcttccagagtcccaatcctttgccctagttctttcactagtttgaaatccaagttcttgccagagtgttggagcaaggcagctgatttgctgcagggatggagaggaccaccgccgcagggttcttttacctgtgccaccagctctggagacatccacacccattcccaggtgtccttccaggcccattgtccacgtcttcaagggggctgcctggataggcgtgtgtgtgtgtagtgcccaggtgtggtggtggcatcctgaagcagtaggaccattagtgtgcgtgcacacccacgtggcacactgtgtggtgaccatggtcatcatagtgggctcacggcggaaacgggatcattcaacctatacaaaggggaccctggataggctgcaaggaaagaggccaagggccaagctgctaagccaaagatggcccctgacacctcccccagccccagcacaccagcctgcacctcttatcccgtgttaagtcctggttccccacctgctgcccctcctcaacacagagcccctcccgccgcctcagacccctgtgcacatccccagggcctcagccgtctcattggtctttattttttattttttttaagatggagtttcgctcttattgcccagactggagtgcagtgatgctatctcggctcactgcaacctttgcctcccaggttcaagtgattctcctgcctcagcctcccaagtagctgggattacaggcgtgcaccaccacgccgggctaaatttttttgtatttttagtagagacggggtttctctatgttggtcaggctgatctcgaactcccgacctcaggtgatccgccagcctcagcctcccaaagtgctgggattacaggcgtgagccactgcacccggctctcactggtcttacgccaccttctggacactccctccttgagggcagaaaggagtcccaggcctgtccctagggacaaggcccagggaagagtgtatttggggagcaggggaggggagggtgttgagaaagctgaactggagtcaatcacccttcccacaaatcaccaaactgctggaactctccagccaaatgctgggagaaggacctggagggtgagtctttgctgacctctctctactctcaggcatgtcttttgtccttttcgtccatctatttctgtctgtcgctcactcgccccgctttctctgtctcaccttcatccactctgcaggcctgctccaccacagccctaatcctctggacgcttgtgtagggcctggggtgaattccctgtcccccatggtacctcgagaggggctggggagctcagcttggtctcagagtctccccaccagatactgtttaaaaaagtagcactgatgtgttttgtaatctgcccctcccagccctccgtggaggctgccagggccttgtacggtaaacctagctgcatgtaatctgtggacaatggcattctctacaatgcaataaaaacaattacccatga

SEQ ID NO: 74 - protein, 678 aa, Homo sapiens
MMVESASETIRSAPSGQNGVGSLSGQADGSSGGATGTTASGTGREVTTGADSNGEMSPAELLHFQQQQALQVARQFLLQQASGLSSPGNNDSKQSASAVQVPVSVAMMSPQMLTPQQMQQILSPPQLQALLQQQQALMLQQEYYKKQQEQLHLQLLTQQQAGKPQPKEALGNKQLAFQQQLLQMQQLQQQHLLNLQRQGLVSLQPNQASGPLQTLPQAAVCPTDLPQLWKGEGAPGQPAEDSVKQEGLDLTGTAATATSFAAPPKVSPPLSHHTLPNGQPTVLTSRRDSSSHEETPGSHPLYGHGECKWPGCETLCEDLGQFIKHLNTEHALDDRSTAQCRVQMQVVQQLEIQLAKESERLQAMMAHLHMRPSEPKPFSQPLNPVPGSSSFSKVTVSAADSFPDGLVHPPTSAAAPVTPLRPPGLGSASLHGGGPARRRSSDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLNSPGMLNPGSASSLLPLSHDDVGAPVEPLPSNGSSSPPRLSPPQYSHQVQVKEEPAEAEEDRQPGPPLGAPNPSASGPPEDRDLEEELPGEELS

SEQ ID NO: 75 - RNA, 5955 nt, Homo sapiens
  source 1..5955: mol_type = mRNA
actcgcagcccaggctcagtcgcagcccctcggagtccggagcccgccgtccctgcggaccggacagtgcggccggcggccggcggccgggacggagcccccagcccgcgaggagggcgcggcgcaggcggcggcggcgcggcggggaggagcccgggccggaaccagggctgggccgggggcggggacgccggggtccgggagcccgggagccggagcgagctgacgagcgtcgcggaaggaccgggaaggaagagcggagccggagcgagccaagcagcccacaaagtgccatgccccggcggggttgaggcgcgcggcggtcggcccggcgcgctgggaggacgcccgggagctgcgcgacgcggggcggcgcggaagggcagccccggcgggcggcggcgggcccgggctgcgaccggagcgagccccatgccccgcgcgggctgacgggccggagcccgcacggagcggccgggcgggacaggtaccgctagagcgacatgatggtggaatctgcctcggagacaatcaggtcggctccatctggtcagaatggcgtgggcagcctctctgggcaagccgatggcagcagcggcggggccacagggacaactgcaagtggcacgggcagggaagtgaccacgggtgcagacagcaatggtgagatgagtcccgcagagctgctgcacttccagcagcaacaggctctccaagtggcccggcagttcctgctgcagcaggcctcaggcctgagctccccagggaacaatgacagcaaacagtctgcctctgctgtgcaggtgcctgtgtcggtggccatgatgtcgccgcagatgcttaccccgcaacagatgcagcagatcctgtcgcccccgcagctgcaggccttgctccagcagcagcaagccctcatgctccagcagctacaggagtactacaagaagcagcaggagcagctccacctgcagctcctcacccagcagcaggctgggaaaccgcagcccaaagaggcactggggaacaagcagctggccttccagcagcagctcctgcaaatgcaacagttgcagcagcagcacctgctcaacctgcagaggcaggggctggtcagcctgcagcccaaccaagcctcggggcccctccagacccttccgcaagctgtttgcccaacagacctgccccagctgtggaagggcgagggtgcccccgggcagcctgccgaggacagcgtcaagcaggaggggctggacctcactggcacggccgccaccgctacctcgtttgccgctccccccaaggtctcaccccccctctcccaccataccctgcccaacggacagcctactgtgctcacatctcggagagacagctcttcccacgaggagacccccggctcccaccccctgtacggacacggagagtgcaagtggccaggctgtgagaccctgtgtgaagacctgggccagtttatcaaacacctcaacacagagcacgccctggatgaccggagtacagcccagtgccgggtacagatgcaggtggtgcagcagctggagatccagctcgccaaggagagcgagcggctgcaggccatgatggcccacctgcacatgcggccctcggagcccaagcccttcagccagccagtgaccgtctctgcagcagactcattcccagatggtctcgtgcaccccccgacctcggccgcagcccctgtcacccctctacggccccctggcctgggctctgcctccctgcatggtgggggcccagcccgtcggagaagcagtgacaagttctgctcccccatctcctcagagctggcccagaatcatgagttctacaagaacgccgacgtccggccccccttcacctacgcctccctcatccgccaggccatcctggaaacccctgacaggcagctgaccctgaatgagatctataactggttcaccaggatgttcgcctatttccgcagaaacactgccacctggaagaacgccgtgcgccacaacctcagcctgcacaagtgcttcgtccgcgtggagaacgtcaagggtgccgtgtggactgtggacgagcgggagtatcagaagcggagaccgccaaagatgacagggagccccaccctggtgaagaacatgatctctggcctcagctatggagcacttaatgccagctaccaggccgccctggccgagagcagcttccccctcctcaacagccctggcatgctgaaccctggctccgccagcagcctgctgcccctcagccacgatgacgtgggtgcccccgtggagccgctgcccagcaacggcagcagcagccctcctcgcctctccccgccccagtacagccaccaggtgcaggtgaaggaggagccagcagaggcagaggaagacaggcagcccgggcctcccctgggcgcccctaaccccagcgcctcggggcctccggaagacagggacctggaggaggagctgccgggagaagaactgtcctaagggcctgtagtgaccggcagggctggggtgagacccctcccttccagaatccaggccccatctcccccaactccacagcccctcccgagcctcaaggcaagtccaggactcagaccggggaggcccgggccagcagctcccagtgtgacctgacaaaaacacgtaggggcagggacggtccccacccccagggacacaacccctggtcttggaccagtagaggacacggagggttcagacccctcctcagaccctccccacatctgaaactgcctccccccaaccaccagcagcagcagggccctcctcccccaccagctctccccacagggcccctcagcatcatggagacccgcaggcggggcttagccacccctcaaacccagggccccctggcacctggctctggccgtgttttctggccagaggcccccactttcctaactcgtgctcccttccgccttcttttccgtactgtgaagaaagaactctccaccccagctcccaccctgccctggcctgggtggaggaactgtgcctccatccccagaagaaacagccccctctgctgctggggtgggactgtctgtgtgccctgtgggggtccgtgtgagcaggcccacctggctccagacccgcccccaacctgagacagaaccaggctgagccaggcctccacccccacccccgtttgctgggggctcctccagccgcccccatgggaagaggcctggtaccgcctcacccacagaggtctgtgccaggtgcgcttctgcaggtggagccaagctctccctgaggccagaggcggggcctgggccgggagcccaggggaaggccaggctggaccccggctccacacccacatccagcctgcaggcctctctgcagtcctctcaccctccctcagctccccttcctctgcagtcaccctcagctccccttccttgcccgcctctccccccgccgccccaccagttaaacggatgaccaaagacctttcttatgccggaagcaaaaaccaaaactttttgttggctttttcctttgtcgcctccccagcacctgccctcccagtctcccaccccggccccaggctggaagccctccctccacttaagttattgttttaaaccaaagtttacagtgtctgttggtggccaagaccttctctctccacccctcctccatccaccctgaggaccctggggctcagtggaggcagggccctgcccccctcccttccgctcctgcccagcctgggggaaggagaaaggaggggagaaagcgggctctcaccccctcaggagtgggcacgggagcccttctccctgaccctgggctgcttcctgggggctctccagacccctctctaggaccaagtcacccgtcgtgctgggagtgtggattctagcaaaagagctggaaaaaagtcagactctccacagaccccctatgggggacccccaactcaaggccaaggactgggcgtatcggatgctcataacacccctggcctggcccctttactgagaagactccttggatatttcccaagaaccccccacatacacccctcacaagccacccctcctgagaggcagggggccctccgccccctccccatgtattccccacctgtgttccgtttgaccagcacagaaatattaaacgtcctctattcaccgggccctgtgtgtgtcaccgaggtgcgggaggggaggagcattaaagctgaaagatcgctctgctcggggagcctgggcagagcagcagcaacgtgagggtcgctgtggtggtggtttctgtgagtggatggaatgagcagccctgcaggggcgctgggcatgtgccctcactgtggacagggcccacccacctccggttcccctgtgcctcctgtcccttccacgcttaaacagggttctctgtcattttcctgttttcttccagagtcccaatcctttgccctagttctttcactagtttgaaatccaagttcttgccagagtgttggagcaaggcagctgatttgctgcagggatggagaggaccaccgccgcagggttcttttacctgtgccaccagctctggagacatccacacccattcccaggtgtccttccaggcccattgtccacgtcttcaagggggctgcctggataggcgtgtgtgtgtgtagtgcccaggtgtggtggtggcatcctgaagcagtaggaccattagtgtgcgtgcacacccacgtggcacactgtgtggtgaccatggtcatcatagtgggctcacggcggaaacgggatcattcaacctatacaaaggggaccctggataggctgcaaggaaagaggccaagggccaagctgctaagccaaagatggcccctgacacctcccccagccccagcacaccagcctgcacctcttatcccgtgttaagtcctggttccccacctgctgcccctcctcaacacagagcccctcccgccgcctcagacccctgtgcacatccccagggcctcagccgtctcattggtctttattttttattttttttaagatggagtttcgctcttattgcccagactggagtgcagtgatgctatctcggctcactgcaacctttgcctcccaggttcaagtgattctcctgcctcagcctcccaagtagctgggattacaggcgtgcaccaccacgccgggctaaatttttttgtatttttagtagagacggggtttctctatgttggtcaggctgatctcgaactcccgacctcaggtgatccgccagcctcagcctcccaaagtgctgggattacaggcgtgagccactgcacccggctctcactggtcttacgccaccttctggacactccctccttgagggcagaaaggagtcccaggcctgtccctagggacaaggcccagggaagagtgtatttggggagcaggggaggggagggtgttgagaaagctgaactggagtcaatcacccttcccacaaatcaccaaactgctggaactctccagccaaatgctgggagaaggacctggagggtgagtctttgctgacctctctctactctcaggcatgtcttttgtccttttcgtccatctatttctgtctgtcgctcactcgccccgctttctctgtctcaccttcatccactctgcaggcctgctccaccacagccctaatcctctggacgcttgtgtagggcctggggtgaattccctgtcccccatggtacctcgagaggggctggggagctcagcttggtctcagagtctccccaccagatactgtttaaaaaagtagcactgatgtgttttgtaatctgcccctcccagccctccgtggaggctgccagggccttgtacggtaaacctagctgcatgtaatctgtggacaatggcattctctacaatgcaataaaaacaattacccatga

SEQ ID NO: 76 - protein, 667 aa, Homo sapiens
MMVESASETIRSAPSGQNGVGSLSGQADGSSGGATGTTASGTGREVTTGADSNGEMSPAELLHFQQQQALQVARQFLLQQASGLSSPGNNDSKQSASAVQVPVSVAMMSPQMLTPQQMQQILSPPQLQALLQQQQALMLQQLQEYYKKQQEQLHLQLLTQQQAGKPQPKEALGNKQLAFQQQLLQMQQLQQQHLLNLQRQGLVSLQPNQASGPLQTLPQAVCPTDLPQLWKGEGAPGQPAEDSVKQEGLDLTGTAATATSFAAPPKVSPPLSHHTLPNGQPTVLTSRRDSSSHEETPGSHPLYGHGECKWPGCETLCEDLGQFIKHLNTEHALDDRSTAQCRVQMQVVQQLEIQLAKESERLQAMMAHLHMRPSEPKPFSQPVTVSAADSFPDGLVHPPTSAAAPVTPLRPPGLGSASLHGGGPARRRSSDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLNSPGMLNPGSASSLLPLSHDDVGAPVEPLPSNGSSSPPRLSPPQYSHQVQVKEEPAEAEEDRQPGPPLGAPNPSASGPPEDRDLEEELPGEELS

SEQ ID NO: 77 - RNA, 5958 nt, Homo sapiens
  source 1..5958: mol_type = mRNA
actcgcagcccaggctcagtcgcagcccctcggagtccggagcccgccgtccctgcggaccggacagtgcggccggcggccggcggccgggacggagcccccagcccgcgaggagggcgcggcgcaggcggcggcggcgcggcggggaggagcccgggccggaaccagggctgggccgggggcggggacgccggggtccgggagcccgggagccggagcgagctgacgagcgtcgcggaaggaccgggaaggaagagcggagccggagcgagccaagcagcccacaaagtgccatgccccggcggggttgaggcgcgcggcggtcggcccggcgcgctgggaggacgcccgggagctgcgcgacgcggggcggcgcggaagggcagccccggcgggcggcggcgggcccgggctgcgaccggagcgagccccatgccccgcgcgggctgacgggccggagcccgcacggagcggccgggcgggacaggtaccgctagagcgacatgatggtggaatctgcctcggagacaatcaggtcggctccatctggtcagaatggcgtgggcagcctctctgggcaagccgatggcagcagcggcggggccacagggacaactgcaagtggcacgggcagggaagtgaccacgggtgcagacagcaatggtgagatgagtcccgcagagctgctgcacttccagcagcaacaggctctccaagtggcccggcagttcctgctgcagcaggcctcaggcctgagctccccagggaacaatgacagcaaacagtctgcctctgctgtgcaggtgcctgtgtcggtggccatgatgtcgccgcagatgcttaccccgcaacagatgcagcagatcctgtcgcccccgcagctgcaggccttgctccagcagcagcaagccctcatgctccagcagctacaggagtactacaagaagcagcaggagcagctccacctgcagctcctcacccagcagcaggctgggaaaccgcagcccaaagaggcactggggaacaagcagctggccttccagcagcagctcctgcaaatgcaacagttgcagcagcagcacctgctcaacctgcagaggcaggggctggtcagcctgcagcccaaccaagcctcggggcccctccagacccttccgcaagcagctgtttgcccaacagacctgccccagctgtggaagggcgagggtgcccccgggcagcctgccgaggacagcgtcaagcaggaggggctggacctcactggcacggccgccaccgctacctcgtttgccgctccccccaaggtctcaccccccctctcccaccataccctgcccaacggacagcctactgtgctcacatctcggagagacagctcttcccacgaggagacccccggctcccaccccctgtacggacacggagagtgcaagtggccaggctgtgagaccctgtgtgaagacctgggccagtttatcaaacacctcaacacagagcacgccctggatgaccggagtacagcccagtgccgggtacagatgcaggtggtgcagcagctggagatccagctcgccaaggagagcgagcggctgcaggccatgatggcccacctgcacatgcggccctcggagcccaagcccttcagccagccagtgaccgtctctgcagcagactcattcccagatggtctcgtgcaccccccgacctcggccgcagcccctgtcacccctctacggccccctggcctgggctctgcctccctgcatggtgggggcccagcccgtcggagaagcagtgacaagttctgctcccccatctcctcagagctggcccagaatcatgagttctacaagaacgccgacgtccggccccccttcacctacgcctccctcatccgccaggccatcctggaaacccctgacaggcagctgaccctgaatgagatctataactggttcaccaggatgttcgcctatttccgcagaaacactgccacctggaagaacgccgtgcgccacaacctcagcctgcacaagtgcttcgtccgcgtggagaacgtcaagggtgccgtgtggactgtggacgagcgggagtatcagaagcggagaccgccaaagatgacagggagccccaccctggtgaagaacatgatctctggcctcagctatggagcacttaatgccagctaccaggccgccctggccgagagcagcttccccctcctcaacagccctggcatgctgaaccctggctccgccagcagcctgctgcccctcagccacgatgacgtgggtgcccccgtggagccgctgcccagcaacggcagcagcagccctcctcgcctctccccgccccagtacagccaccaggtgcaggtgaaggaggagccagcagaggcagaggaagacaggcagcccgggcctcccctgggcgcccctaaccccagcgcctcggggcctccggaagacagggacctggaggaggagctgccgggagaagaactgtcctaagggcctgtagtgaccggcagggctggggtgagacccctcccttccagaatccaggccccatctcccccaactccacagcccctcccgagcctcaaggcaagtccaggactcagaccggggaggcccgggccagcagctcccagtgtgacctgacaaaaacacgtaggggcagggacggtccccacccccagggacacaacccctggtcttggaccagtagaggacacggagggttcagacccctcctcagaccctccccacatctgaaactgcctccccccaaccaccagcagcagcagggccctcctcccccaccagctctccccacagggcccctcagcatcatggagacccgcaggcggggcttagccacccctcaaacccagggccccctggcacctggctctggccgtgttttctggccagaggcccccactttcctaactcgtgctcccttccgccttcttttccgtactgtgaagaaagaactctccaccccagctcccaccctgccctggcctgggtggaggaactgtgcctccatccccagaagaaacagccccctctgctgctggggtgggactgtctgtgtgccctgtgggggtccgtgtgagcaggcccacctggctccagacccgcccccaacctgagacagaaccaggctgagccaggcctccacccccacccccgtttgctgggggctcctccagccgcccccatgggaagaggcctggtaccgcctcacccacagaggtctgtgccaggtgcgcttctgcaggtggagccaagctctccctgaggccagaggcggggcctgggccgggagcccaggggaaggccaggctggaccccggctccacacccacatccagcctgcaggcctctctgcagtcctctcaccctccctcagctccccttcctctgcagtcaccctcagctccccttccttgcccgcctctccccccgccgccccaccagttaaacggatgaccaaagacctttcttatgccggaagcaaaaaccaaaactttttgttggctttttcctttgtcgcctccccagcacctgccctcccagtctcccaccccggccccaggctggaagccctccctccacttaagttattgttttaaaccaaagtttacagtgtctgttggtggccaagaccttctctctccacccctcctccatccaccctgaggaccctggggctcagtggaggcagggccctgcccccctcccttccgctcctgcccagcctgggggaaggagaaaggaggggagaaagcgggctctcaccccctcaggagtgggcacgggagcccttctccctgaccctgggctgcttcctgggggctctccagacccctctctaggaccaagtcacccgtcgtgctgggagtgtggattctagcaaaagagctggaaaaaagtcagactctccacagaccccctatgggggacccccaactcaaggccaaggactgggcgtatcggatgctcataacacccctggcctggcccctttactgagaagactccttggatatttcccaagaaccccccacatacacccctcacaagccacccctcctgagaggcagggggccctccgccccctccccatgtattccccacctgtgttccgtttgaccagcacagaaatattaaacgtcctctattcaccgggccctgtgtgtgtcaccgaggtgcgggaggggaggagcattaaagctgaaagatcgctctgctcggggagcctgggcagagcagcagcaacgtgagggtcgctgtggtggtggtttctgtgagtggatggaatgagcagccctgcaggggcgctgggcatgtgccctcactgtggacagggcccacccacctccggttcccctgtgcctcctgtcccttccacgcttaaacagggttctctgtcattttcctgttttcttccagagtcccaatcctttgccctagttctttcactagtttgaaatccaagttcttgccagagtgttggagcaaggcagctgatttgctgcagggatggagaggaccaccgccgcagggttcttttacctgtgccaccagctctggagacatccacacccattcccaggtgtccttccaggcccattgtccacgtcttcaagggggctgcctggataggcgtgtgtgtgtgtagtgcccaggtgtggtggtggcatcctgaagcagtaggaccattagtgtgcgtgcacacccacgtggcacactgtgtggtgaccatggtcatcatagtgggctcacggcggaaacgggatcattcaacctatacaaaggggaccctggataggctgcaaggaaagaggccaagggccaagctgctaagccaaagatggcccctgacacctcccccagccccagcacaccagcctgcacctcttatcccgtgttaagtcctggttccccacctgctgcccctcctcaacacagagcccctcccgccgcctcagacccctgtgcacatccccagggcctcagccgtctcattggtctttattttttattttttttaagatggagtttcgctcttattgcccagactggagtgcagtgatgctatctcggctcactgcaacctttgcctcccaggttcaagtgattctcctgcctcagcctcccaagtagctgggattacaggcgtgcaccaccacgccgggctaaatttttttgtatttttagtagagacggggtttctctatgttggtcaggctgatctcgaactcccgacctcaggtgatccgccagcctcagcctcccaaagtgctgggattacaggcgtgagccactgcacccggctctcactggtcttacgccaccttctggacactccctccttgagggcagaaaggagtcccaggcctgtccctagggacaaggcccagggaagagtgtatttggggagcaggggaggggagggtgttgagaaagctgaactggagtcaatcacccttcccacaaatcaccaaactgctggaactctccagccaaatgctgggagaaggacctggagggtgagtctttgctgacctctctctactctcaggcatgtcttttgtccttttcgtccatctatttctgtctgtcgctcactcgccccgctttctctgtctcaccttcatccactctgcaggcctgctccaccacagccctaatcctctggacgcttgtgtagggcctggggtgaattccctgtcccccatggtacctcgagaggggctggggagctcagcttggtctcagagtctccccaccagatactgtttaaaaaagtagcactgatgtgttttgtaatctgcccctcccagccctccgtggaggctgccagggccttgtacggtaaacctagctgcatgtaatctgtggacaatggcattctctacaatgcaataaaaacaattacccatga

SEQ ID NO: 78 - protein, 668 aa, Homo sapiens
MMVESASETIRSAPSGQNGVGSLSGQADGSSGGATGTTASGTGREVTTGADSNGEMSPAELLHFQQQQALQVARQFLLQQASGLSSPGNNDSKQSASAVQVPVSVAMMSPQMLTPQQMQQILSPPQLQALLQQQQALMLQQLQEYYKKQQEQLHLQLLTQQQAGKPQPKEALGNKQLAFQQQLLQMQQLQQQHLLNLQRQGLVSLQPNQASGPLQTLPQAAVCPTDLPQLWKGEGAPGQPAEDSVKQEGLDLTGTAATATSFAAPPKVSPPLSHHTLPNGQPTVLTSRRDSSSHEETPGSHPLYGHGECKWPGCETLCEDLGQFIKHLNTEHALDDRSTAQCRVQMQVVQQLEIQLAKESERLQAMMAHLHMRPSEPKPFSQPVTVSAADSFPDGLVHPPTSAAAPVTPLRPPGLGSASLHGGGPARRRSSDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLNSPGMLNPGSASSLLPLSHDDVGAPVEPLPSNGSSSPPRLSPPQYSHQVQVKEEPAEAEEDRQPGPPLGAPNPSASGPPEDRDLEEELPGEELS

SEQ ID NO: 79 - RNA, 5994 nt, Homo sapiens
  source 1..5994: mol_type = mRNA
actcgcagcccaggctcagtcgcagcccctcggagtccggagcccgccgtccctgcggaccggacagtgcggccggcggccggcggccgggacggagcccccagcccgcgaggagggcgcggcgcaggcggcggcggcgcggcggggaggagcccgggccggaaccagggctgggccgggggcggggacgccggggtccgggagcccgggagccggagcgagctgacgagcgtcgcggaaggaccgggaaggaagagcggagccggagcgagccaagcagcccacaaagtgccatgccccggcggggttgaggcgcgcggcggtcggcccggcgcgctgggaggacgcccgggagctgcgcgacgcggggcggcgcggaagggcagccccggcgggcggcggcgggcccgggctgcgaccggagcgagccccatgccccgcgcgggctgacgggccggagcccgcacggagcggccgggcgggacaggtaccgctagagcgacatgatggtggaatctgcctcggagacaatcaggtcggctccatctggtcagaatggcgtgggcagcctctctgggcaagccgatggcagcagcggcggggccacagggacaactgcaagtggcacgggcagggaagtgaccacgggtgcagacagcaatggtgagatgagtcccgcagagctgctgcacttccagcagcaacaggctctccaagtggcccggcagttcctgctgcagcaggcctcaggcctgagctccccagggaacaatgacagcaaacagtctgcctctgctgtgcaggtgcctgtgtcggtggccatgatgtcgccgcagatgcttaccccgcaacagatgcagcagatcctgtcgcccccgcagctgcaggccttgctccagcagcagcaagccctcatgctccagcagctacaggagtactacaagaagcagcaggagcagctccacctgcagctcctcacccagcagcaggctgggaaaccgcagcccaaagaggcactggggaacaagcagctggccttccagcagcagctcctgcaaatgcaacagttgcagcagcagcacctgctcaacctgcagaggcaggggctggtcagcctgcagcccaaccaagcctcggggcccctccagacccttccgcaagcagctgtttgcccaacagacctgccccagctgtggaagggcgagggtgcccccgggcagcctgccgaggacagcgtcaagcaggaggggctggacctcactggcacggccgccaccgctacctcgtttgccgctccccccaaggtctcaccccccctctcccaccataccctgcccaacggacagcctactgtgctcacatctcggagagacagctcttcccacgaggagacccccggctcccaccccctgtacggacacggagagtgcaagtggccaggctgtgagaccctgtgtgaagacctgggccagtttatcaaacacctcaacacagagcacgccctggatgaccggagtacagcccagtgccgggtacagatgcaggtggtgcagcagctggagatccagctcgccaaggagagcgagcggctgcaggccatgatggcccacctgcacatgcggccctcggagcccaagcccttcagccagccactgaacccggtccccggctcctcctcattctccaaggtgaccgtctctgcagcagactcattcccagatggtctcgtgcaccccccgacctcggccgcagcccctgtcacccctctacggccccctggcctgggctctgcctccctgcatggtgggggcccagcccgtcggagaagcagtgacaagttctgctcccccatctcctcagagctggcccagaatcatgagttctacaagaacgccgacgtccggccccccttcacctacgcctccctcatccgccaggccatcctggaaacccctgacaggcagctgaccctgaatgagatctataactggttcaccaggatgttcgcctatttccgcagaaacactgccacctggaagaacgccgtgcgccacaacctcagcctgcacaagtgcttcgtccgcgtggagaacgtcaagggtgccgtgtggactgtggacgagcgggagtatcagaagcggagaccgccaaagatgacagggagccccaccctggtgaagaacatgatctctggcctcagctatggagcacttaatgccagctaccaggccgccctggccgagagcagcttccccctcctcaacagccctggcatgctgaaccctggctccgccagcagcctgctgcccctcagccacgatgacgtgggtgcccccgtggagccgctgcccagcaacggcagcagcagccctcctcgcctctccccgccccagtacagccaccaggtgcaggtgaaggaggagccagcagaggcagaggaagacaggcagcccgggcctcccctgggcgcccctaaccccagcgcctcggggcctccggaagacagggacctggaggaggagctgccgggagaagaactgtcctaagggcctgtagtgaccggcagggctggggtgagacccctcccttccagaatccaggccccatctcccccaactccacagcccctcccgagcctcaaggcaagtccaggactcagaccggggaggcccgggccagcagctcccagtgtgacctgacaaaaacacgtaggggcagggacggtccccacccccagggacacaacccctggtcttggaccagtagaggacacggagggttcagacccctcctcagaccctccccacatctgaaactgcctccccccaaccaccagcagcagcagggccctcctcccccaccagctctccccacagggcccctcagcatcatggagacccgcaggcggggcttagccacccctcaaacccagggccccctggcacctggctctggccgtgttttctggccagaggcccccactttcctaactcgtgctcccttccgccttcttttccgtactgtgaagaaagaactctccaccccagctcccaccctgccctggcctgggtggaggaactgtgcctccatccccagaagaaacagccccctctgctgctggggtgggactgtctgtgtgccctgtgggggtccgtgtgagcaggcccacctggctccagacccgcccccaacctgagacagaaccaggctgagccaggcctccacccccacccccgtttgctgggggctcctccagccgcccccatgggaagaggcctggtaccgcctcacccacagaggtctgtgccaggtgcgcttctgcaggtggagccaagctctccctgaggccagaggcggggcctgggccgggagcccaggggaaggccaggctggaccccggctccacacccacatccagcctgcaggcctctctgcagtcctctcaccctccctcagctccccttcctctgcagtcaccctcagctccccttccttgcccgcctctccccccgccgccccaccagttaaacggatgaccaaagacctttcttatgccggaagcaaaaaccaaaactttttgttggctttttcctttgtcgcctccccagcacctgccctcccagtctcccaccccggccccaggctggaagccctccctccacttaagttattgttttaaaccaaagtttacagtgtctgttggtggccaagaccttctctctccacccctcctccatccaccctgaggaccctggggctcagtggaggcagggccctgcccccctcccttccgctcctgcccagcctgggggaaggagaaaggaggggagaaagcgggctctcaccccctcaggagtgggcacgggagcccttctccctgaccctgggctgcttcctgggggctctccagacccctctctaggaccaagtcacccgtcgtgctgggagtgtggattctagcaaaagagctggaaaaaagtcagactctccacagaccccctatgggggacccccaactcaaggccaaggactgggcgtatcggatgctcataacacccctggcctggcccctttactgagaagactccttggatatttcccaagaaccccccacatacacccctcacaagccacccctcctgagaggcagggggccctccgccccctccccatgtattccccacctgtgttccgtttgaccagcacagaaatattaaacgtcctctattcaccgggccctgtgtgtgtcaccgaggtgcgggaggggaggagcattaaagctgaaagatcgctctgctcggggagcctgggcagagcagcagcaacgtgagggtcgctgtggtggtggtttctgtgagtggatggaatgagcagccctgcaggggcgctgggcatgtgccctcactgtggacagggcccacccacctccggttcccctgtgcctcctgtcccttccacgcttaaacagggttctctgtcattttcctgttttcttccagagtcccaatcctttgccctagttctttcactagtttgaaatccaagttcttgccagagtgttggagcaaggcagctgatttgctgcagggatggagaggaccaccgccgcagggttcttttacctgtgccaccagctctggagacatccacacccattcccaggtgtccttccaggcccattgtccacgtcttcaagggggctgcctggataggcgtgtgtgtgtgtagtgcccaggtgtggtggtggcatcctgaagcagtaggaccattagtgtgcgtgcacacccacgtggcacactgtgtggtgaccatggtcatcatagtgggctcacggcggaaacgggatcattcaacctatacaaaggggaccctggataggctgcaaggaaagaggccaagggccaagctgctaagccaaagatggcccctgacacctcccccagccccagcacaccagcctgcacctcttatcccgtgttaagtcctggttccccacctgctgcccctcctcaacacagagcccctcccgccgcctcagacccctgtgcacatccccagggcctcagccgtctcattggtctttattttttattttttttaagatggagtttcgctcttattgcccagactggagtgcagtgatgctatctcggctcactgcaacctttgcctcccaggttcaagtgattctcctgcctcagcctcccaagtagctgggattacaggcgtgcaccaccacgccgggctaaatttttttgtatttttagtagagacggggtttctctatgttggtcaggctgatctcgaactcccgacctcaggtgatccgccagcctcagcctcccaaagtgctgggattacaggcgtgagccactgcacccggctctcactggtcttacgccaccttctggacactccctccttgagggcagaaaggagtcccaggcctgtccctagggacaaggcccagggaagagtgtatttggggagcaggggaggggagggtgttgagaaagctgaactggagtcaatcacccttcccacaaatcaccaaactgctggaactctccagccaaatgctgggagaaggacctggagggtgagtctttgctgacctctctctactctcaggcatgtcttttgtccttttcgtccatctatttctgtctgtcgctcactcgccccgctttctctgtctcaccttcatccactctgcaggcctgctccaccacagccctaatcctctggacgcttgtgtagggcctggggtgaattccctgtcccccatggtacctcgagaggggctggggagctcagcttggtctcagagtctccccaccagatactgtttaaaaaagtagcactgatgtgttttgtaatctgcccctcccagccctccgtggaggctgccagggccttgtacggtaaacctagctgcatgtaatctgtggacaatggcattctctacaatgcaataaaaacaattacccatga

SEQ ID NO: 80 - protein, 680 aa, Homo sapiens
MMVESASETIRSAPSGQNGVGSLSGQADGSSGGATGTTASGTGREVTTGADSNGEMSPAELLHFQQQQALQVARQFLLQQASGLSSPGNNDSKQSASAVQVPVSVAMMSPQMLTPQQMQQILSPPQLQALLQQQQALMLQQLQEYYKKQQEQLHLQLLTQQQAGKPQPKEALGNKQLAFQQQLLQMQQLQQQHLLNLQRQGLVSLQPNQASGPLQTLPQAAVCPTDLPQLWKGEGAPGQPAEDSVKQEGLDLTGTAATATSFAAPPKVSPPLSHHTLPNGQPTVLTSRRDSSSHEETPGSHPLYGHGECKWPGCETLCEDLGQFIKHLNTEHALDDRSTAQCRVQMQVVQQLEIQLAKESERLQAMMAHLHMRPSEPKPFSQPLNPVPGSSSFSKVTVSAADSFPDGLVHPPTSAAAPVTPLRPPGLGSASLHGGGPARRRSSDKFCSPISSELAQNHEFYKNADVRPPFTYASLIRQAILETPDRQLTLNEIYNWFTRMFAYFRRNTATWKNAVRHNLSLHKCFVRVENVKGAVWTVDEREYQKRRPPKMTGSPTLVKNMISGLSYGALNASYQAALAESSFPLLNSPGMLNPGSASSLLPLSHDDVGAPVEPLPSNGSSSPPRLSPPQYSHQVQVKEEPAEAEEDRQPGPPLGAPNPSASGPPEDRDLEEELPGEELS

(19) United States
(12) Patent Application Publication
(10) Pub. No.: US 2025/0297281 A1
(43) Pub. Date: Sep. 25, 2025

(54) COMPOSITIONS AND METHODS FOR RETINAL NEURON GENERATION

(71) Applicant: UNIVERSITY OF UTAH RESEARCH FOUNDATION, Salt Lake City, UT (US)

(72) Inventors: Jianmin Zhang, Salt Lake City, UT (US); Monica L. Vetter, Salt Lake City, UT (US); Fei Chang, Salt Lake City, UT (US); Jacqueline M. Roberts, Salt Lake City, UT (US)

(21) Appl. No.: 18/861,832

(22) PCT Filed: May 26, 2023
(86) PCT No.: PCT/US2023/067549

§ 371 Date: Oct. 30, 2024

Related U.S. Application Data
(60) Provisional application No. 63/346,428, filed on May 27, 2022.
(60) Provisional application No. 63/439,941, filed on Jan. 19, 2023.

(51) Int. Cl. C12N 15/86; A61K 48/00; C07K 14/47

(52) U.S. Cl. CPC C12N 15/86; A61K 48/005; C07K 14/4702; C12N 2710/10032; C12N 2710/10045

(57) ABSTRACT

Described herein are compositions and methods for inducing retinal regeneration in a subject. Also described herein are compositions and methods for treating, preventing, reducing the likelihood of having, reducing the severity of, and/or slowing the progression of a retinal degenerative disease, retinal damage, or retinal blindness in a subject. In some embodiments, the compositions and methods may comprise a pharmaceutical composition comprising a Foxp gene expression vector comprising a polynucleotide sequence encoding a Foxp polypeptide, functional variant thereof, or fragment thereof.

### CROSS-REFERENCE TO RELATED APPLICATIONS

This application claims priority to U.S. Provisional Patent Application Nos. 63/346,428, filed on May 27, 2022, and 63/439,941, filed on Jan. 19, 2023, each of which is incorporated by reference herein in its entirety.

### FEDERALLY SPONSORED RESEARCH

This invention was made with government support under grant number EY012274 awarded by the National Institutes of Health. The government has certain rights in the invention.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “026389-9335-WO01_sequence_listing_xml_11 May 2023.xml,” was created on May 11, 2023, contains 80 sequences, has a file size of 328 Kbytes, and is incorporated by reference in its entirety into the specification.

### BACKGROUND

Temporal patterning of progenitors is critical for generating a diversity of cell types during development of the nervous system, driving the differentiation of neurons and glia in specific proportions in a defined sequence. Changes in neural progenitor competence govern the ability of progenitors to differentiate into diverse neuronal and glial cell types. A major challenge is determining how regulation of temporal competence in the vertebrate central nervous system (CNS) is coordinated by transcriptional programs and epigenetic modifications.

This problem is well defined in the developing retina, since lineage analysis has shown that multipotent retinal progenitor cells (RPCs) progress through temporally regulated competence states during which they generate distinct cell types in a defined sequence that is conserved across species. In mouse, early-born cell types (retinal ganglion cells (RGCs), cone photoreceptors, horizontal cells, some amacrine cells) are generated embryonically, while late-born cell types (most rod photoreceptors, remaining amacrine cells, bipolar cells, Müller glia) are generated postnatally, with precise regulation of cell fate specification. Cell-intrinsic processes primarily regulate the timing of cell genesis, with late progenitors restricted from generating earlier cell types. Müller glia have a similar transcriptional profile as late progenitors, and in lower vertebrates have the potential to reprogram and regenerate retinal cells, although this is restricted in mammals. Recent single cell analysis of mouse and human retinal development has revealed a distinct shift in gene expression and chromatin accessibility from early to late-stage RPCs, consistent with a change in competence to generate early versus late-born retinal cell types. Multiple temporal identity factors and regulators have been shown to regulate RPC competence and timing of retinal cell production, including those with similarity to temporal identity factors first identified in Drosophila such as Ikaros family (Ikzf1 and Ikzf4) and Casz1, as well as other transcription factors such as FoxN4, Pou2f1/Pou2f2, Lhx2 and NFI factors, along with microRNA regulation through Dicer. Additionally, epigenetic regulators including Jarid2 have been defined to regulate early RPC competence. However, whether additional factors regulate RPC competence, particularly for generation of early-born retinal cell types, and whether such factors may facilitate the ability of Müller glia to produce retinal neurons are still unclear.

What is needed are novel compositions and methods for inducing retinal neuron generation. Such compositions and methods would be useful in a variety of therapeutic and clinical applications including the treatment and/or prevention of retinal degenerative disease, retinal damage, or retinal blindness in a subject.

### SUMMARY

One embodiment described herein is a method for inducing retinal regeneration in a subject, the method comprising: administering to the subject a therapeutically effective amount of a pharmaceutical composition comprising: a Foxp gene expression vector comprising a polynucleotide sequence encoding a Foxp polypeptide, functional variant thereof, or fragment thereof. In one aspect, the Foxp gene expression vector is a Foxp1, Foxp2, or Foxp4 gene expression vector encoding a Foxp1, Foxp2, or Foxp4 polypeptide, functional variant thereof, or fragment thereof. In another aspect, the Foxp gene expression vector is a Foxp1 gene expression vector encoding a Foxp1 polypeptide, functional variant thereof, or fragment thereof. In another aspect, the polynucleotide sequence encoding the Foxp polypeptide, functional variant thereof, or fragment thereof has at least 90-99% identity to any one of the odd-numbered sequences from SEQ ID NO: 1-79. In another aspect, the polynucleotide sequence encoding the Foxp polypeptide, functional variant thereof, or fragment thereof is selected from any one of the odd-numbered sequences from SEQ ID NO: 1-79. In another aspect, the polynucleotide sequence encoding the Foxp polypeptide, functional variant thereof, or fragment thereof has at least 90-99% identity to any one of the odd-numbered sequences from SEQ ID NO: 25-79. In another aspect, the polynucleotide sequence encoding the Foxp polypeptide, functional variant thereof, or fragment thereof is selected from any one of the odd-numbered sequences from SEQ ID NO: 25-79. In another aspect, the Foxp polypeptide, functional variant thereof, or fragment thereof comprises an amino acid sequence having at least 90-99% identity to any one of the even-numbered sequences from SEQ ID NO: 2-80. In another aspect, the Foxp polypeptide, functional variant thereof, or fragment thereof comprises an amino acid sequence selected from any one of the even-numbered sequences from SEQ ID NO: 2-80. In another aspect, the Foxp polypeptide, functional variant thereof, or fragment thereof comprises an amino acid sequence having at least 90-99% identity to any one of the even-numbered sequences from SEQ ID NO: 26-80. In another aspect, the Foxp polypeptide, functional variant thereof, or fragment thereof comprises an amino acid sequence selected from any one of the even-numbered sequences from SEQ ID NO: 26-80. In another aspect, the pharmaceutical composition is administered to a retina of the subject by intravitreal or subretinal injection. In another aspect, the Foxp gene expression vector is selected from a viral vector, a lentiviral vector, a plasmid expression vector, an adeno-associated virus (AAV) vector, a recombinant AAV (rAAV) vector, a single-stranded AAV vector, a double-stranded AAV vector, a self-complementary AAV (scAAV) vector, or combinations thereof. The method of claim 13, wherein the Foxp gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof. In another aspect, the Foxp gene expression vector comprises a promoter sequence operably linked to the polynucleotide sequence encoding the Foxp polypeptide, functional variant thereof, or fragment thereof. In another aspect, the promoter sequence is a retinal-specific promoter sequence or a Müller glia (MG)-specific promoter sequence. In another aspect, the pharmaceutical composition further comprises one or more nanoparticles for administration of the Foxp gene expression vector to the subject. In another aspect, the one or more nanoparticles comprise lipid-based nanoparticles, peptide-based nanoparticles, or a combination thereof. In another aspect, the Foxp polypeptide, functional variant thereof, or fragment thereof reprograms MG to generate MG-derived functional retinal neurons. In another aspect, the MG-derived functional retinal neurons comprise retinal ganglion cells and cone photoreceptors that are generated during early stages of retina development. In another aspect, the number of MG-derived functional retinal neurons in the subject is increased as compared to a baseline level of functional retinal neurons in the subject prior to administration. In another aspect, the pharmaceutical composition does not comprise a histone deacetylase (HDAC) inhibitor. In another aspect, the pharmaceutical composition does not comprise a Jak/STAT signaling pathway inhibitor. In another aspect, the subject has one or more of a retinal degenerative disease, retinal damage, or retinal blindness. In another aspect, the subject has a retinal degenerative disease comprising age-related macular degeneration (AMD), retinitis pigmentosa (RP), diabetic retinopathy (DR), central retinal artery occlusion (CRAG), vitreoretinopathy, glaucoma, Usher syndrome, optic neuropathy, optic nerve injury, or combinations thereof. In another aspect, the therapeutically effective amount of the pharmaceutical composition is administered to the subject as a single dose or as a plurality of doses.

Another embodiment described herein is a method for treating, preventing, reducing the likelihood of having, reducing the severity of, and/or slowing the progression of a retinal degenerative disease, retinal damage, or retinal blindness in a subject, the method comprising: administering to the subject a therapeutically effective amount of a pharmaceutical composition comprising: a Foxp gene expression vector comprising a polynucleotide sequence encoding a Foxp polypeptide, functional variant thereof, or fragment thereof. In one aspect, the Foxp gene expression vector is a Foxp1, Foxp2, or Foxp4 gene expression vector encoding a Foxp1, Foxp2, or Foxp4 polypeptide, functional variant thereof, or fragment thereof. In another aspect, the Foxp gene expression vector is a Foxp1 gene expression vector encoding a Foxp1 polypeptide, functional variant thereof, or fragment thereof.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows scRNA-seq expression of early and late RPC Genes, related to FIGS. 2 and 5. Genes cited in this study as having early or late gene expression are plotted. At each age, E11-P5, the proportion of RPCs expressing the gene and average gene expression are represented by circle size and color, respectively. This data is represented both scaled (z-score across ages) and unscaled. scRNA-seq data is from Clark et al., Neuron 102:1111-1126 e1115 (2019).

FIG. 2 shows that Foxp1 regulates the generation of early-born retinal cell types. FIG. 2A (top row) shows Foxp1 protein expression in wild type retinas at E12.5, E14.5, E16.5 and P0. FIG. 2A (bottom row) shows Foxp1 expression in the central retina from E14.5 Foxp1f^{l+} (control) and Six3-Cre Foxp1^{fl/fl} (Foxp1 cKO) littermates. FIG. 2B-C show that Foxp1 cKO retina had reduced Brn3a-labeled RGCs (FIG. 2B Brn3a row, left and right columns, FIG. 2C #Brn3a+ RGC/field graph), reduced Calbindin-labeled horizontal cells (FIG. 2B Calbindin row, left and right columns, FIG. 2C #Calbindin+ HC/field graph), and reduced Rxrγ-labeled cone photoreceptors (FIG. 2B #Rxrγ row, left and right columns, FIG. 2C #Rxrγ+ Cone/field graph) within central dorsal retinas at P0. Regions of interest in FIG. 2B were marked by white brackets. FIG. 2D shows a cropped volcano plot of Foxp1 cKO E16.5 bulk RNA-seq results. Red dots represent significantly differentially expressed genes. P-adj<0.05. NBL: neuroblastic layer. *p<0.05, **p<0.01. ns: not significant. Scale bar: 100 μm.

FIG. 3 shows that sustaining Foxp1 in RPCs increased early-born retinal cells and decreased late-born cells. FIG. 3A shows Foxp1 expression in P0 Foxp1^{tg/+} (control) and Six3-Cre Foxp1^{tg/+} (Foxp1 cTG) littermates (top row). Foxp1 cTG showed increased Brn3a-labeled RGCs (FIG. 3A, row Brn3a, FIG. 3C, #Brn3a+ RGC/field graph), Onecut2-labeled horizontal cells (FIG. 3A, row Onecut2, FIG. 3C, #Onecut2+HC/field graph), and Rxrγ-labeled cone photoreceptors (FIG. 3A, row Rxrγ, FIG. 3C, #Rxrγ+ Cone/field graph) within central ventral retinas at P0. FIG. 3B shows Foxp1 expression and Brn3a-labeled RGCs in P0 Six3-Cre Foxp1tg/tg retina. Foxp1 cTG showed decreased Vsx2-labeled bipolar cells (FIG. 3D, row Vsx2, FIG. 3E #Vsx2+BC/field graph), and reduced thickness of Rhodopsin-labeled rod photoreceptor layer (FIG. 3D, row Hoechst Rhodopsin, FIG. 3E Rhodopsin layer thickness graph (μm)). Arrowheads in FIG. 3D (Hoechst Rhodopsin row) indicate abnormal structure in the subretinal region. Foxp1 cTG showed reduced Lhx2 and Cralbp co-labeled Müller glia in the INL (FIG. 3E #Lhx2+ Cralbp+ MG INL/field graph) and increased Müller glia in the ONL and OPL within the central retina at P10 (FIG. 3E #Lhx2+ Cralbp+ MG ONL & OPL/field graph). Arrowheads in FIG. 3D (Hoechst Lhx2 Cralbp row) indicated mislocated Müller glia in the ONL and OPL. This shows that Foxp1 expression alters normal Müller glia differentiation. Foxp1 cTG with EdU administration at E18.5 showed increased EdU labeled Brn3a within the central retina at P0 (FIG. 3F, FIG. 3G schematic diagram and #EdU+ Brn3a+/field graph). Zoomed-in images were from single z-slice. Foxp1 cTG retina showed increased cell cycle exit of progenitors (FIG. 3G % EdU+ PCNA−/EdU+ graph). NBL: neuroblastic layer; GCL: ganglion cell layer; ONL: outer nuclear layer; INL: inner nuclear layer. *p<0.05, **p<0.01, ***p<0.001, by Student's T test. Scale bar: 100 μm.

FIG. 4 shows that retinal neurogenesis is affected in Foxp1 cTG, related to FIG. 3. Foxp1 immunostaining on retinal cross sections from P0 Foxp1 cTG and control littermates confirmed that Foxp1 expression is increased in Foxp1 cTG (FIG. 4A). FIG. 4B shows Foxp1 and Brn3a immunostaining showing mosaic levels of Foxp1 expression, with higher levels coinciding with increased Brn3a labeled cells. White dash lines demarcate higher Foxp1 expression region. FIG. 4C shows rosette structures that are visible in the outer nuclear layer in a subset of Foxp1 cTG retinal sections. ONL: outer nuclear layer. Scale bar: 100 μm.

FIG. 5 shows that ectopic Foxp1 expression results in increased early RPC gene expression. UMAP dimensional reduction of scRNA-seq in P0 Foxp1 cTG and littermate control is shown colored by annotated cell types in FIG. 5A and shown by genotype in FIG. 5B. FIG. 5C shows violin plots of normalized transcript counts of differentially expressed genes in Foxp1 control and cTG RPCs, colored by genotype. FIG. 5D shows a volcano plot of all expressed genes, with genes with significantly changed expression in Foxp1 cTG shown in red. P-adj<0.01, average log 2 Fold Change >0.1. See also Table 2. Genes are plotted such that those with increased expression in the Foxp1 cTG are on the right. FIG. 5E shows a Venn diagram comparing differentially expressed genes common to Jarid2 cKO and Foxp1 cTG RPCs. RPC marker genes are shown. FIG. 5F shows GSEA of MSigDB mouse hallmark gene sets in Jarid2 cKO and Foxp1 cTG RPCs. Significantly enriched gene sets with adjusted p-value <0.05 in Foxp1 cTG (brown) and Jarid2 cKO (green) are marked with an asterisk and categorized by biological process.

FIG. 6 shows that re-expressing Foxp1 in late RPCs induces the generation of cone photoreceptors during the postnatal period after the normal competence window has closed. FIG. 6A shows that Foxp1 was re-expressed in late RPCs with tamoxifen administration at P0 and P1, as detected by immunostaining for GFP (FIG. 6A GFP row) and ectopic Foxp1 transgene (FIG. 6A Foxp1 row) in the NBL at P3 in Foxp1^{tg/+} and Rax-Cre^{ERT2} Foxp1^{tg/+} littermates. Scale bar: 100 μm. NBL, neuroblastic layer. FIG. 6B shows that Foxp1 expression in late RPCs, as detected by Flag antibody, generates Rxrγ and mCAR-labeled cone photoreceptors detected in P10 Rax-Cre^{ERT2} Foxp1 cTG retina (arrowheads). Scale bar: 100 μm. ONL, outer nuclear layer.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “gene,” “nucleic acid,” “nucleotide,” “polynucleotide,” “oligonucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. Nucleic acids may be single stranded or double stranded or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

As used herein, “variants” can include, but are not limited to, those that include conservative amino acid (AA) substitution, SNP variants, degenerate variants, and biologically active portions of a gene. A “degenerate variant” as used herein refers to a variant that has a mutated nucleotide sequence, but still encodes the same polypeptide due to the redundancy of the genetic code. There are 20 naturally occurring amino acids; however, some of these share similar characteristics. For example, leucine and isoleucine are both aliphatic, branched, and hydrophobic. Similarly, aspartic acid and glutamic acid are both small and negatively charged. Conservative substitutions in proteins often have a smaller effect on function than non-conservative mutations. Although there are many ways to classify amino acids, they are often sorted into six main groups on the basis of their structure and the general chemical characteristics of their R groups. A mutation among the same class of amino acids is considered a conservative amino acid substitution.

The term “functional” when used in conjunction with “variant” or “fragment” refers to an entity or molecule which possess a biological activity that is substantially similar to a biological activity of the entity or molecule of which it is a variant or fragment thereof. In accordance with the present invention, a Foxp family polypeptide may be modified, for example, to facilitate or improve identification, expression, isolation, bioavailability, storage, and/or administration, so long as such modifications do not reduce its function to an unacceptable level. For example, in one non-limiting embodiment, a Foxp family polypeptide may include a C-terminal or N-terminal flag tag (i.e., a DYKDDDDK amino acid tag), or any other types of tags well-known in the art, and these tags do not affect the function of the Foxp polypeptide. In various embodiments, a Foxp polypeptide functional variant or fragment thereof has at least 10%, 20%, 30%, 40%, 50%, 60%, 70%, 80%, 90%, 95%, or 99% of the function of a full-length wildtype Foxp polypeptide.

As used herein, “substantial identity” of polynucleotide sequences means that a polynucleotide comprises a sequence that has at least 25% sequence identity compared to a reference sequence as determined using programs known in the art (e.g., Basic Local Alignment Search Tool (BLAST)). In preferred embodiments, percent identity can be any integer from 25% to 100%. More preferred embodiments include polynucleotide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. These values can be appropriately adjusted to determine corresponding identity of proteins encoded by two nucleotide sequences by taking into account codon degeneracy, amino acid similarity, reading frame positioning, and the like. Accordingly, polynucleotides of the present invention encoding a protein or polypeptide of the present invention include nucleic acid sequences that have substantial identity to the nucleic acid sequences that encode the proteins or polypeptides of the present invention. Polynucleotides encoding a polypeptide comprising an amino acid sequence that has at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference polypeptide sequence are also preferred.

As used herein, “substantial identity” of amino acid sequences (and of polypeptides having these amino acid sequences) means that an amino acid sequence comprises a sequence that has at least 25% sequence identity compared to a reference sequence as determined using programs known in the art (e.g., BLAST). In preferred embodiments, percent identity can be any integer from 25% to 100%. More preferred embodiments include amino acid or polypeptide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” share amino acid sequences except that residue positions which are not identical may differ by one or more conservative amino acid changes, as described above. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Exemplary conservative amino acid substitution groups include valine-leucine-isoleucine, phenylalanine-tyrosine, lysine-arginine, alanine-valine, aspartic acid-glutamic acid, and asparagine-glutamine. Accordingly, polypeptides or proteins, encoded by the polynucleotides of the present invention, include amino acid sequences that have substantial identity to the amino acid sequences of the reference polypeptide sequences.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, pharmaceutical compositions, or mixtures thereof, that provide a pharmacological, often beneficial, effect. In some embodiments, disclosed compositions may further comprise one or more pharmaceutically acceptable carriers or excipients. Example carriers may include, but are not limited to, liposomes, polymeric micelles, microspheres, microparticles, dendrimers, or nanoparticles. For example, in some embodiments of the present invention, disclosed pharmaceutical compositions may further comprise one or more nanoparticles for administration of a Foxp gene expression vector to a subject. In some embodiments, the one or more nanoparticles may comprise lipid-based nanoparticles, peptide-based nanoparticles, or a combination thereof.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the term “administering” refers to the placement of an agent or a composition as disclosed herein into a subject by a method or route which results in at least partial localization of the agents or composition at a desired site. “Route of administration” may refer to any administration pathway known in the art, including but not limited to oral, intravenous (IV), topical, aerosol, nasal, via inhalation, anal, intra-anal, peri-anal, transmucosal, transdermal, parenteral, enteral, or local. “Parenteral” refers to a route of administration that is generally associated with injection, including intracranial, intraventricular, intrathecal, epidural, intradural, intraorbital, intravitreal, subretinal, infusion, intracapsular, intracardiac, intradermal, intramuscular, intraperitoneal, intrapulmonary, intraspinal, intrasternal, intrathecal, intrauterine, intravascular, intravenous (IV), intraarterial, subarachnoid, subcapsular, subcutaneous, transmucosal, or transtracheal. Via the parenteral route, the agent or composition may be in the form of solutions or suspensions for IV infusion or IV injection, or as lyophilized powders. Via the enteral route, the agent or composition can be in the form of capsules, gel capsules, tablets, sugar-coated tablets, syrups, suspensions, solutions, powders, granules, emulsions, microspheres or nanospheres or lipid vesicles or polymer vesicles allowing controlled release. Via the topical route, the agent or composition can be in the form of aerosol, lotion, cream, gel, ointment, suspensions, solutions, or emulsions. In one embodiment, the agent or composition may be provided in a powder form and mixed with a liquid, such as water, to form a beverage. In accordance with the present invention, “administering” can be self-administering. For example, it is considered “administering” when a subject consumes a composition as disclosed herein.

As used herein, “contacting” refers to contacting a target cell with an agent (e.g., a Foxp gene expression vector) using any method that is suitable for placing the agent on, in, or adjacent to a target cell. For example, when the cells are in vitro, contacting the cells with the agent can comprise adding the agent to culture medium containing the cells. For example, when the cells are in vivo, contacting the cells with the agent can comprise administering the agent to a subject.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments. In some embodiments of the present invention, a subject is in need of treatment if the subject is suffering from, or at risk of suffering from, one or more of a retinal degenerative disease, retinal damage, or retinal blindness. In some embodiments, the subject may be suffering from, or at risk of suffering from, a retinal degenerative disease comprising age-related macular degeneration (AMD), retinitis pigmentosa (RP), diabetic retinopathy (DR), central retinal artery occlusion (CRAG), vitreoretinopathy, glaucoma, Usher syndrome, optic neuropathy, optic nerve injury, or combinations thereof.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic manner. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest. In one embodiment of the present invention, a method of treating, preventing, reducing the likelihood of having, reducing the severity of, and/or slowing the progression of a retinal degenerative disease, retinal damage, or retinal blindness in a subject is described.

In some embodiments, a subject may be administered a single dose of the disclosed pharmaceutical compositions. In other embodiments, the subject may be administered a plurality of doses of the disclosed pharmaceutical compositions over a period of time. For example, in various nonlimiting embodiments, a pharmaceutical composition as described herein may be administered to a subject once a day (SID/QD), twice a day (BID), three times a day (TID), four times a day (QID), or more, so as to administer a therapeutically effective amount of the pharmaceutical composition to the subject, where the therapeutically effective amount is any one or more of the doses described herein. In some embodiments, a pharmaceutical composition as described herein is administered to a subject 1-3 times per day, 1-7 times per week, 1-9 times per month, 1-12 times per year, or more. In other embodiments, a pharmaceutical composition as described herein is administered for about 1-10 days, 10-20 days, 20-30 days, 30-40 days, 40-50 days, 50-60 days, 60-70 days, 70-80 days, 80-90 days, 90-100 days, 1-6 months, 6-12 months, 1-5 years, or more. In various embodiments, a pharmaceutical composition as described herein is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, 900-1000 mg/kg, or a combination thereof.

The actual dosing regimen can depend upon many factors, including but not limited to the judgment of a trained physician, the overall condition of the subject, and the specific type of disease or disorder in the subject. The actual dosage can also depend on the determined experimental effectiveness of the specific pharmaceutical composition that is administered. For example, the dosage may be determined based on in vitro responsiveness of relevant cultured cells, or in vivo responses observed in appropriate animal models or human studies.

As used herein, “sample” or “target sample” refers to any sample in which the presence and/or level of a target analyte or target biomarker is to be detected or determined. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological or bodily fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

During development of the CNS, transitions in competence underlie the ability of progenitors to generate a diversity of neurons and glia. In mammalian retina there are two major transcriptional states of progenitor cells, which in mouse give rise to early-born cell types embryonically and late-born cell types largely postnatally. The transition from early to late progenitor competence is regulated by the transcription factor Foxp1. Artificially sustained Foxp1 expression resulted in extended production of early cell types, with single cell RNA-seq analysis showing increased representation of early progenitor genes at later stages. Further, re-expression of Foxp1 at postnatal stages was sufficient to re-establish competence to produce early retinal cell types. Conversely, conditional loss of Foxp1 was shown to reduce the generation of early-born retinal cell types and promote late progenitor gene expression. Together, these observations establish Foxp1 as a key regulator of early temporal patterning and generation of early born retinal neurons including retinal ganglion cells and cone photoreceptors.

The Foxp subfamily of forkhead box (FOX) proteins, which consists of four members-Foxp1, Foxp2, Foxp3, and Foxp4—is characterized on the basis of its members containing a C2H2-type zinc finger domain and a leucine zipper motif (i.e., coiled-coil domain) in addition to a forkhead domain at the C-terminus. The C-terminal location of the forkhead domain is an atypical feature in the Foxp subfamily, as most other Fox family members have this domain in the N-terminal portion. Among the subfamily members, Foxp1, Foxp2, and Foxp4 are highly homologous (showing more than 60% identity at the amino acid level); in particular, their forkhead domains show approximately 80% identity at the amino acid level.

Various embodiments of the present invention provide a pharmaceutical composition comprising a Foxp gene expression vector comprising a polynucleotide sequence encoding a Foxp polypeptide, functional variant thereof, or fragment thereof (i.e., a Foxp gene therapy) for inducing retinal regeneration in a subject, or for treating, preventing, reducing the likelihood of having, reducing the severity of, and/or slowing the progression of a retinal degenerative disease, retinal damage, or retinal blindness in a subject. In some embodiments, various gene expression vectors as described herein are used to produce various Foxp polypeptides, functional variants thereof, or fragments thereof. In various embodiments, the gene expression vector is a plasmid. In various embodiments, the gene expression vector is a viral vector, adeno-associated virus (AAV) vector, recombinant AAV (rAAV) vector, single-stranded AAV vector, double-stranded AAV vector, self-complementary AAV (scAAV) vector, or a combination thereof. In various embodiments, the gene expression vector is a polynucleotide or a virus particle. In various embodiments, the serotype of the virus particle is AAV1, AAV2, AAV3, AAV4, AAV5, AAV6, AAV7, AAV8, AAV9, AAV10, AAV11, AAV12, or a hybrid serotype thereof. In various embodiments, the Foxp gene expression vector comprises a promoter sequence operably linked to the polynucleotide sequence encoding the Foxp polypeptide, functional variant thereof, or fragment. In various embodiments, the promoter sequence is a retinal-specific promoter sequence or a Müller glia (MG)-specific promoter sequence.

In various embodiments, the Foxp gene therapy is derived from a mammal. In various embodiments, the Foxp gene therapy is derived from a primate, for example, a human, a chimpanzee, a gorilla, or a monkey. In various embodiments, the Foxp gene therapy is derived from a rodent, for example, a mouse, a rat, or a guinea pig. In various embodiments, the Foxp gene therapy is derived from a horse, a goat, a donkey, a cow, a bull, or a pig. In various embodiments, the Foxp gene therapy is derived from a chicken, a duck, a frog, a dog, a cat, or a rabbit.

In some embodiments of the present invention, the Foxp gene expression vector is a Foxp1, Foxp2, or Foxp4 gene expression vector encoding a Foxp1, Foxp2, or Foxp4 polypeptide, functional variant thereof, or fragment thereof. Non-limiting exemplary Foxp polynucleotide and amino acid sequences of the present invention are included below in Table 1.

TABLE 1
Foxp Polynucleotide and Amino Acid Sequences

| | | Sequence | NIH GenBank | SEQ |
|---|---|---|---|---|
| Foxp Isoform | Organism | Type | Accession No. | ID NO |
| Foxp1, transcript variant 2 | Mouse | mRNA | NM_001197321.1 | 1 |
| Foxp1, isoform 2 | Mouse | Protein | NP_001184250.1 | 2 |
| Foxp1, transcript variant 3 | Mouse | mRNA | NM_001197322.1 | 3 |
| Foxp1, isoform 3 | Mouse | Protein | NP_001184251.1 | 4 |
| Foxp1, transcript variant 4 | Mouse | mRNA | NM_001347345.1 | 5 |
| Foxp1, isoform 4 | Mouse | Protein | NP_001334274.1 | 6 |
| Foxp1, transcript variant 1 | Mouse | mRNA | NM_053202.2 | 7 |
| Foxp1, isoform 1 | Mouse | Protein | NP_444432.1 | 8 |
| Foxp2, transcript variant 1 | Mouse | mRNA | NM_053242.4 | 9 |
| Foxp2, isoform 1 | Mouse | Protein | NP_444472.2 | 10 |
| Foxp2, transcript variant 3 | Mouse | mRNA | NM_001286607.1 | 11 |
| Foxp2, isoform 2 | Mouse | Protein | NP_001273536.1 | 12 |
| Foxp2, transcript variant 2 | Mouse | mRNA | NM_212435.1 | 13 |
| Foxp2, isoform 1 | Mouse | Protein | NP_997600.1 | 14 |
| Foxp4, transcript variant 1 | Mouse | mRNA | NM_001110824.2 | 15 |
| Foxp4, isoform 1 | Mouse | Protein | NP_001104294.1 | 16 |
| Foxp4, transcript variant 2 | Mouse | mRNA | NM_001110825.2 | 17 |
| Foxp4, isoform 2 | Mouse | Protein | NP_001104295.1 | 18 |
| Foxp4, transcript variant 5 | Mouse | mRNA | NM_001403970.1 | 19 |
| Foxp4, isoform 5 | Mouse | Protein | NP_001390899.1 | 20 |
| Foxp4, transcript variant 4 | Mouse | mRNA | NM_001403969.1 | 21 |
| Foxp4, isoform 4 | Mouse | Protein | NP_001390898.1 | 22 |
| Foxp4, transcript variant 3 | Mouse | mRNA | NM_028767.3 | 23 |
| Foxp4, isoform 3 | Mouse | Protein | NP_083043.2 | 24 |
| Foxp1, transcript variant 5 | Human | mRNA | NM_001244812.3 | 25 |
| Foxp1, isoform e | Human | Protein | NP_001231741.1 | 26 |
| Foxp1, transcript variant 9 | Human | mRNA | NM_001244816.2 | 27 |
| Foxp1, isoform a | Human | Protein | NP_001231745.1 | 28 |
| Foxp1, transcript variant 11 | Human | mRNA | NM_001349338.3 | 29 |
| Foxp1, isoform a | Human | Protein | NP_001336267.1 | 30 |
| Foxp1, transcript variant 3 | Human | mRNA | NM_001244808.3 | 31 |
| Foxp1, isoform c | Human | Protein | NP_001231737.1 | 32 |
| Foxp1, transcript variant 14 | Human | mRNA | NM_001349341.3 | 33 |
| Foxp1, isoform j | Human | Protein | NP_001336270.1 | 34 |
| Foxp1, transcript variant 13 | Human | mRNA | NM_001349340.3 | 35 |
| Foxp1, isoform a | Human | Protein | NP_001336269.1 | 36 |
| Foxp1, transcript variant 1 | Human | mRNA | NM_032682.6 | 37 |
| Foxp1, isoform a | Human | Protein | NP_116071.2 | 38 |
| Foxp1, transcript variant 4 | Human | mRNA | NM_001244810.2 | 39 |
| Foxp1, isoform d | Human | Protein | NP_001231739.1 | 40 |
| Foxp1, transcript variant 6 | Human | mRNA | NM_001244813.3 | 41 |
| Foxp1, isoform f | Human | Protein | NP_001231742.1 | 42 |
| Foxp1, transcript variant 17 | Human | mRNA | NM_001349344.3 | 43 |
| Foxp1, isoform i | Human | Protein | NP_001336273.1 | 44 |
| Foxp1, transcript variant 8 | Human | mRNA | NM_001244815.2 | 45 |
| Foxp1, isoform f | Human | Protein | NP_001231744.2 | 46 |
| Foxp1, transcript variant 20 | Human | mRNA | NM_001370548.1 | 47 |
| Foxp1, isoform k | Human | Protein | NP_001357477.1 | 48 |
| Foxp1, transcript variant 10 | Human | mRNA | NM_001349337.2 | 49 |
| Foxp1, isoform i | Human | Protein | NP_001336266.2 | 50 |
| Foxp1, transcript variant 15 | Human | mRNA | NM_001349342.3 | 51 |
| Foxp1, isoform f | Human | Protein | NP_001336271.1 | 52 |
| Foxp1, transcript variant 16 | Human | mRNA | NM_001349343.3 | 53 |
| Foxp1, isoform i | Human | Protein | NP_001336272.1 | 54 |
| Foxp1, transcript variant 7 | Human | mRNA | NM_001244814.3 | 55 |
| Foxp1, isoform a | Human | Protein | NP_001231743.1 | 56 |
| Foxp2, transcript variant 2 | Human | mRNA | NM_148898.4 | 57 |
| Foxp2, isoform II | Human | Protein | NP_683696.2 | 58 |
| Foxp2, transcript variant 5 | Human | mRNA | NM_001172766.3 | 59 |
| Foxp2, isoform V | Human | Protein | NP_001166237.1 | 60 |
| Foxp2, transcript variant 1 | Human | mRNA | NM_014491.4 | 61 |
| Foxp2, isoform I | Human | Protein | NP_055306.1 | 62 |
| Foxp2, transcript variant 3 | Human | mRNA | NM_148899.3 | 63 |
| Foxp2, isoform III | Human | Protein | NP_683697.2 | 64 |
| Foxp2, transcript variant 6 | Human | mRNA | NM_001172767.2 | 65 |
| Foxp2, isoform VI | Human | Protein | NP_001166238.1 | 66 |
| Foxp2, transcript variant 4 | Human | mRNA | NM_148900.4 | 67 |
| Foxp2, isoform IV | Human | Protein | NP_683698.2 | 68 |
| Foxp4, transcript variant 6 | Human | mRNA | NM_001405826.1 | 69 |
| Foxp4, isoform 6 | Human | Protein | NP_001392755.1 | 70 |
| Foxp4, transcript variant 5 | Human | mRNA | NM_001405825.1 | 71 |
| Foxp4, isoform 5 | Human | Protein | NP_001392754.1 | 72 |
| Foxp4, transcript variant 3 | Human | mRNA | NM_001012427.2 | 73 |
| Foxp4, isoform 3 | Human | Protein | NP_001012427.1 | 74 |
| Foxp4, transcript variant 2 | Human | mRNA | NM_138457.3 | 75 |
| Foxp4, isoform 2 | Human | Protein | NP_612466.1 | 76 |
| Foxp4, transcript variant 4 | Human | mRNA | NM_001405824.1 | 77 |
| Foxp4, isoform 4 | Human | Protein | NP_001392753.1 | 78 |
| Foxp4, transcript variant 1 | Human | mRNA | NM_001012426.2 | 79 |
| Foxp4, isoform 1 | Human | Protein | NP_001012426.1 | 80 |

Described herein are studies investigating the effects of loss or overexpression of Foxp1 in mouse models. Mouse (i.e., murine) Foxp1 knockout studies disrupted the expression of all known mouse Foxp1 isoforms. Foxp1 overexpression studies then used a flag-tagged version of mouse Foxp1 mRNA (SEQ ID NO: 1) to overexpress and restore levels of mouse Foxp1 protein (SEQ ID NO: 2).

Alternative mouse Foxp1 protein isoforms (SEQ ID NO: 4, 6, and 8) encoded from corresponding mouse Foxp1 mRNA transcripts (SEQ ID NO: 3, 5, and 7) each have at least 85% protein sequence conservation compared to the tested overexpressed Foxp1 isoform of SEQ ID NO: 2. Further, all four of the mouse Foxp1 protein isoforms (SEQ ID NO: 2, 4, 6, and 8) contain identical predicted coiled-coil and forkhead domains, and thus are predicted to be functionally redundant.

Similarly, all mouse Foxp2 protein isoforms (SEQ ID NO: 10, 12, and 14) encoded from corresponding mouse Foxp2 mRNA transcripts (SEQ ID NO: 9, 11, and 13), and all mouse Foxp4 protein isoforms (SEQ ID NO: 16, 18, 20, 22, and 24) encoded from corresponding mouse Foxp4 mRNA transcripts (SEQ ID NO: 15, 17, 19, 21, and 23), show significant homology across the functional coiled-coil and forkhead domains to the mouse Foxp1 protein isoform (SEQ ID NO: 2) that was overexpressed in the studies presented herein.

When compared to the tested mouse Foxp1 protein isoform (SEQ ID NO: 2), various human Foxp1 protein isoforms (even-numbered sequences from SEQ ID NO: 26-56) have 95.65% conserved sequence identity across the entire coiled-coil functional domain. Additionally, all of the human Foxp1 protein isoforms included in Table 1 have 100% conserved sequence identity across 97% of the forkhead functional domain, except for SEQ ID NO: 40, which has 82.14% conserved sequence identity across 98% of the forkhead functional domain.

Further, the human Foxp2 protein isoforms (even-numbered sequences from SEQ ID NO: 58-68) have 88.10% conserved sequence identity across 98% of the forkhead functional domain and 85.51% conserved sequence identity across the entire coiled-coil functional domain. Additionally, all of the human Foxp4 protein isoforms (even-numbered sequences from SEQ ID NO: 70-80) have 90.59% sequence identity across the entire forkhead functional domain and 81.16% conserved sequence identity across the entire coiled-coil functional domain.

Based on the high conservation of protein sequence as described above, all of the Foxp isoform mRNA sequences included in Table 1 (odd-numbered sequences from SEQ ID NO: 3-79) are predicted to have a conserved sequence as compared to the tested mouse Foxp1 mRNA transgene (SEQ ID NO: 1).

It should be understood that other Foxp isoform sequences having high homology and substantial sequence identity, as defined herein, to the sequences included in Table 1 may also be suitable for use in the disclosed invention. For example, this may include additional Foxp1, Foxp2, or Foxp4 isoform sequences of human or mouse origin, or Foxp1, Foxp2, or Foxp4 isoform sequences from any organism including, but not limited to, other mammals; primates such as a human, a chimpanzee, a gorilla, or a monkey; a rodent such as a mouse, a rat, or a guinea pig; a horse, a goat, a donkey, a cow, a bull, or a pig; or a chicken, a duck, a frog, a dog, a cat, or a rabbit.

One embodiment described herein is a method for inducing retinal regeneration in a subject, the method may comprise: administering to the subject a therapeutically effective amount of a pharmaceutical composition comprising: a Foxp gene expression vector comprising a polynucleotide sequence encoding a Foxp polypeptide, functional variant thereof, or fragment thereof. In one aspect, the Foxp gene expression vector may be a Foxp1, Foxp2, or Foxp4 gene expression vector encoding a Foxp1, Foxp2, or Foxp4 polypeptide, functional variant thereof, or fragment thereof. In another aspect, the Foxp gene expression vector may be a Foxp1 gene expression vector encoding a Foxp1 polypeptide, functional variant thereof, or fragment thereof. In another aspect, the polynucleotide sequence encoding the Foxp polypeptide, functional variant thereof, or fragment thereof may have at least 90-99% identity to any one of the odd-numbered sequences from SEQ ID NO: 1-79. In another aspect, the polynucleotide sequence encoding the Foxp polypeptide, functional variant thereof, or fragment thereof may be selected from any one of the odd-numbered sequences from SEQ ID NO: 1-79. In another aspect, the polynucleotide sequence encoding the Foxp polypeptide, functional variant thereof, or fragment thereof may have at least 90-99% identity to any one of the odd-numbered sequences from SEQ ID NO: 25-79. In another aspect, the polynucleotide sequence encoding the Foxp polypeptide, functional variant thereof, or fragment thereof may be selected from any one of the odd-numbered sequences from SEQ ID NO: 25-79. In another aspect, the Foxp polypeptide, functional variant thereof, or fragment thereof may comprise an amino acid sequence having at least 90-99% identity to any one of the even-numbered sequences from SEQ ID NO: 2-80. In another aspect, the Foxp polypeptide, functional variant thereof, or fragment thereof may comprise an amino acid sequence selected from any one of the even-numbered sequences from SEQ ID NO: 2-80. In another aspect, the Foxp polypeptide, functional variant thereof, or fragment thereof may comprise an amino acid sequence having at least 90-99% identity to any one of the even-numbered sequences from SEQ ID NO: 26-80. In another aspect, the Foxp polypeptide, functional variant thereof, or fragment thereof may comprise an amino acid sequence selected from any one of the even-numbered sequences from SEQ ID NO: 26-80.

In another aspect, the pharmaceutical composition may be administered to a retina of the subject by intravitreal or subretinal injection. In another aspect, the Foxp gene expression vector may be selected from a viral vector, a lentiviral vector, a plasmid expression vector, an adeno-associated virus (AAV) vector, a recombinant AAV (rAAV) vector, a single-stranded AAV vector, a double-stranded AAV vector, a self-complementary AAV (scAAV) vector, or combinations thereof. In another aspect, the Foxp gene expression vector may be an AAV vector of a serotype selected from AAV1, AAV2, AAV3, AAV4, AAV5, AAV6, AAV7, AAV8, AAV9, AAV10, AAV11, AAV12, or a hybrid serotype thereof. In another aspect, the Foxp gene expression vector may comprise a promoter sequence operably linked to the polynucleotide sequence encoding the Foxp polypeptide, functional variant thereof, or fragment thereof. In another aspect, the promoter sequence may be a retinal-specific promoter sequence or a Müller glia (MG)-specific promoter sequence. In another aspect, the pharmaceutical composition may further comprise one or more nanoparticles for administration of the Foxp gene expression vector to the subject. In another aspect, the one or more nanoparticles may comprise lipid-based nanoparticles, peptide-based nanoparticles, or a combination thereof.

In another aspect, the Foxp polypeptide, functional variant thereof, or fragment thereof may reprogram MG to generate MG-derived functional retinal neurons. In another aspect, the MG-derived functional retinal neurons may comprise retinal ganglion cells and cone photoreceptors that are normally only generated during early stages of retina development. In another aspect, the number of MG-derived functional retinal neurons in the subject may be increased as compared to a baseline level of functional retinal neurons in the subject prior to administration.

In another aspect, the pharmaceutical composition may not comprise a histone deacetylase (HDAC) inhibitor. In another aspect, the pharmaceutical composition may not comprise a Jak/STAT signaling pathway inhibitor.

In another aspect, the subject may have one or more of a retinal degenerative disease, retinal damage, or retinal blindness. In another aspect, the subject may have a retinal degenerative disease comprising age-related macular degeneration (AMD), retinitis pigmentosa (RP), diabetic retinopathy (DR), central retinal artery occlusion (CRAG), vitreoretinopathy, glaucoma, Usher syndrome, optic neuropathy, optic nerve injury, or combinations thereof. In another aspect, the therapeutically effective amount of the pharmaceutical composition may be administered to the subject as a single dose or as a plurality of doses.

Another embodiment described herein is a method for treating, preventing, reducing the likelihood of having, reducing the severity of, and/or slowing the progression of a retinal degenerative disease, retinal damage, or retinal blindness in a subject, the method may comprise: administering to the subject a therapeutically effective amount of a pharmaceutical composition comprising: a Foxp gene expression vector comprising a polynucleotide sequence encoding a Foxp polypeptide, functional variant thereof, or fragment thereof. In one aspect, the Foxp gene expression vector may be a Foxp1, Foxp2, or Foxp4 gene expression vector encoding a Foxp1, Foxp2, or Foxp4 polypeptide, functional variant thereof, or fragment thereof. In another aspect, the Foxp gene expression vector may be a Foxp1 gene expression vector encoding a Foxp1 polypeptide, functional variant thereof, or fragment thereof.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for inducing retinal regeneration in a subject, the method comprising:
   administering to the subject a therapeutically effective amount of a pharmaceutical composition comprising:
   a Foxp gene expression vector comprising a polynucleotide sequence encoding a Foxp polypeptide, functional variant thereof, or fragment thereof.
Clause 2. The method of clause 1, wherein the Foxp gene expression vector is a Foxp1, Foxp2, or Foxp4 gene expression vector encoding a Foxp1, Foxp2, or Foxp4 polypeptide, functional variant thereof, or fragment thereof.
Clause 3. The method of clause 1 or 2, wherein the Foxp gene expression vector is a Foxp1 gene expression vector encoding a Foxp1 polypeptide, functional variant thereof, or fragment thereof.
Clause 4. The method of any one of clauses 1-3, wherein the polynucleotide sequence encoding the Foxp polypeptide, functional variant thereof, or fragment thereof has at least 90-99% identity to any one of the odd-numbered sequences from SEQ ID NO: 1-79.
Clause 5. The method of any one of clauses 1-4, wherein the polynucleotide sequence encoding the Foxp polypeptide, functional variant thereof, or fragment thereof is selected from any one of the odd-numbered sequences from SEQ ID NO: 1-79.
Clause 6. The method of any one of clauses 1-5, wherein the polynucleotide sequence encoding the Foxp polypeptide, functional variant thereof, or fragment thereof has at least 90-99% identity to any one of the odd-numbered sequences from SEQ ID NO: 25-79.
Clause 7. The method of any one of clauses 1-6, wherein the polynucleotide sequence encoding the Foxp polypeptide, functional variant thereof, or fragment thereof is selected from any one of the odd-numbered sequences from SEQ ID NO: 25-79.
Clause 8. The method of any one of clauses 1-7, wherein the Foxp polypeptide, functional variant thereof, or fragment thereof comprises an amino acid sequence having at least 90-99% identity to any one of the even-numbered sequences from SEQ ID NO: 2-80.
Clause 9. The method of any one of clauses 1-8, wherein the Foxp polypeptide, functional variant thereof, or fragment thereof comprises an amino acid sequence selected from any one of the even-numbered sequences from SEQ ID NO: 2-80.
Clause 10. The method of any one of clauses 1-9, wherein the Foxp polypeptide, functional variant thereof, or fragment thereof comprises an amino acid sequence having at least 90-99% identity to any one of the even-numbered sequences from SEQ ID NO: 26-80.
Clause 11. The method of any one of clauses 1-10, wherein the Foxp polypeptide, functional variant thereof, or fragment thereof comprises an amino acid sequence selected from any one of the even-numbered sequences from SEQ ID NO: 26-80.
Clause 12. The method of any one of clauses 1-11, wherein the pharmaceutical composition is administered to a retina of the subject by intravitreal or subretinal injection.
Clause 13. The method of any one of clauses 1-12, wherein the Foxp gene expression vector is selected from a viral vector, a lentiviral vector, a plasmid expression vector, an adeno-associated virus (AAV) vector, a recombinant AAV (rAAV) vector, a single-stranded AAV vector, a double-stranded AAV vector, a self-complementary AAV (scAAV) vector, or combinations thereof.
Clause 14. The method of any one of clauses 1-13, wherein the Foxp gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof.
Clause 15. The method of any one of clauses 1-14, wherein the Foxp gene expression vector comprises a promoter sequence operably linked to the polynucleotide sequence encoding the Foxp polypeptide, functional variant thereof, or fragment thereof.
Clause 16. The method of any one of clauses 1-15, wherein the promoter sequence is a retinal-specific promoter sequence or a Müller glia (MG)-specific promoter sequence.
Clause 17. The method of any one of clauses 1-16, wherein the pharmaceutical composition further comprises one or more nanoparticles for administration of the Foxp gene expression vector to the subject.
Clause 18. The method of any one of clauses 1-17, wherein the one or more nanoparticles comprise lipid-based nanoparticles, peptide-based nanoparticles, or a combination thereof.
Clause 19. The method of any one of clauses 1-18, wherein the Foxp polypeptide, functional variant thereof, or fragment thereof reprograms MG to generate MG-derived functional retinal neurons.
Clause 20. The method of any one of clauses 1-19, wherein the MG-derived functional retinal neurons comprise retinal ganglion cells and cone photoreceptors that are generated during early stages of retina development.
Clause 21. The method of any one of clauses 1-19, wherein the number of MG-derived functional retinal neurons in the subject is increased as compared to a baseline level of functional retinal neurons in the subject prior to administration.
Clause 22. The method of any one of clauses 1-21, wherein the pharmaceutical composition does not comprise a histone deacetylase (HDAC) inhibitor.
Clause 23. The method of any one of clauses 1-22, wherein the pharmaceutical composition does not comprise a Jak/STAT signaling pathway inhibitor.
Clause 24. The method of any one of clauses 1-23, wherein the subject has one or more of a retinal degenerative disease, retinal damage, or retinal blindness.
Clause 25. The method any one of clauses 1-24, wherein the subject has a retinal degenerative disease comprising age-related macular degeneration (AMD), retinitis pigmentosa (RP), diabetic retinopathy (DR), central retinal artery occlusion (CRAG), vitreoretinopathy, glaucoma, Usher syndrome, optic neuropathy, optic nerve injury, or combinations thereof.
Clause 26. The method of any one of clauses 1-25, wherein the therapeutically effective amount of the pharmaceutical composition is administered to the subject as a single dose or as a plurality of doses.
Clause 27. A method for treating, preventing, reducing the likelihood of having, reducing the severity of, and/or slowing the progression of a retinal degenerative disease, retinal damage, or retinal blindness in a subject, the method comprising:
   administering to the subject a therapeutically effective amount of a pharmaceutical composition comprising:
   a Foxp gene expression vector comprising a polynucleotide sequence encoding a Foxp polypeptide, functional variant thereof, or fragment thereof.
Clause 28. The method of clause 27, wherein the Foxp gene expression vector is a Foxp1, Foxp2, or Foxp4 gene expression vector encoding a Foxp1, Foxp2, or Foxp4 polypeptide, functional variant thereof, or fragment thereof.
Clause 29. The method of clause 27 or 28, wherein the Foxp gene expression vector is a Foxp1 gene expression vector encoding a Foxp1 polypeptide, functional variant thereof, or fragment thereof.

### EXAMPLES

### Example 1

### Mouse Strains

In order to generate retinal Foxp1 conditional transgenic mice (Foxp1 cTG), Foxp1a transgenic mice (Wang et al., Nature Immunology 15:667-675 (2014)) were crossed with Six3-Cre mice (Furuta et al., Genesis 26:130-132 (2000)) or Rax-Cre^{ERT2} (Pak et al., PloS One 9: e90381 (2014)). For Rax-Cre^{ERT2} animals, Cre activity was induced by intraperitoneal injection of 75 μg Tamoxifen per gram of body weight to pregnant females at E10.5. Tamoxifen was dissolved in corn oil (Sigma-Aldrich, C8267) at a concentration of 10 mg/mL.

To generate Foxp1 conditional knockout mice (Foxp1 cKO), Foxp1 floxed mice (Feng et al., Blood 115:510-518 (2010)) were crossed with Six3-Cre mice to generate Six3-Cre; Foxp1^{fl/fl}.

The morning a plug was detected was considered embryonic day 0.5. All animals were treated within the guidelines of the University of Utah Institutional Animal Care and Use Committee (IACUC) and all experiments were IACUC approved.

### Tissue Processing

For embryonic and P0 retinal cryostat sectioning, whole heads were collected. For P10/P14 retinal cryostat sections, eyeballs were enucleated from euthanized mice and the dorsal eye marked by a cauterizer. Tissue was pre-fixed in 4% PFA for 15 minutes and a small incision was made on the cornea. Tissue was further fixed in 4% PFA for 30 minutes, washed 3 times for 20 minutes in PBS, then submerged in 10% and 20% sucrose in PBS at 4° C. Tissue was embedded in OCT compound (Tissue-Tek, Cat #27050) and stored at −80° C. Coronal cryostat sections were made at 16 μm thickness and later processed for RNA in situ hybridization and immunostaining with antibodies listed in the table as previously described (See Zhang et al., Dev. Biol. 403:128-138 (2015)).

### Edu Incorporation

Pregnant females were given intraperitoneal injection of EdU (2 μL 10 mM EdU/gram of body weight, Invitrogen #C10637) at specific time points. EdU staining was performed on cryostat section using Click-iT plus EdU Imaging Kits prior to immunostaining with antibodies.

### Confocal Microscopy

Confocal images were acquired on an inverted Nikon A1R Confocal Microscope. Images were acquired at 20× objective with a 3× digital zoom to obtain a 0.2 μm pixel resolution. Stacks through the Z-plane were taken at 0.8 μm step distance through at total of about 16 μm, covering the entire thickness of the retina. Image acquisition settings were consistent across ages and genotypes.

### Bulk RNA Sequencing

For bulk RNA-seq analysis of Foxp1 cKO, total RNA from E16.5 retinas was isolated using RNeasy Plus Mini Kit. Three biological replicates of each: Six3-Cre; Foxp1^{fl/fl} animals and Foxp1f/fl controls, all from the same litter, were used for RNA sequencing.

Library generation, sequencing, and alignment were performed by the Huntsman Cancer Institute High-Throughput Genomics and Bioinformatics Analysis Shared Resource. Total RNA concentration and quality were measured by RNA ScreenTape Assay (Agilent Technologies, 5067-5576, 5067-5577). All samples had a RIN value of 9.9 or better. The NEBNext Ultra II Directional RNA Library Prep Kit for Illumina with NEBNext rRNA Depletion Kit v2 (E7400) was used to generate libraries which were qualified by D1000 ScreenTape Assay (Agilent Technologies, 5067-5582, 5067-5583) and quantified with a Kapa Library Quant Kit (Kapa Biosystems, KK4824). Samples were sequenced on an Illumina NovaSeq 6000 instrument using the NovaSeq XP workflow (20043131). A 150×150 cycle paired end sequence run was performed using a NovaSeq 6000 S4 reagent Kit v1.5 (20028312).

To clean up the sequencing data for analysis, overlapping reads were grouped by BBTools (v38.34) clumpify script, specified to remove duplicate reads and optical duplicates at a max distance of 12000. Illumina adapters were trimmed with cutadapt (v2.8) using a minimum read length of 20 and minimum overlap between read and adaptor of 6, then aligned to mouse Ensembl genome annotation release 102 with STAR (v2.7.6a) in two pass mode. Reads were assigned to the target that had the largest overlap and uniquely aligned, reverse stranded, reads were counted with featureCounts (v1.5.1). Genes with fewer than 5 counts in every sample were eliminated and differentially expressed genes were identified using DESeq2 (v1.32.0). In addition to the variable of interest: genotype, the additional variable: sex, was included in the DESeq2 design formula to mitigate its effect on the results.

### Single Cell Library Preparation and Sequencing

For single cell analysis in Foxp1 cTG, P0 retinas from female littermates with the genotypes Foxp1^{tg/+}; Six3-Cre (Foxp1 cTG) and Foxp1^{tg/+} (control) were used. Two freshly dissected retinas from one individual animal were pooled for each sample and dissociated in PBS, 50 mM HEPES, 0.05 mg/mL DNase I and 0.025 mg/mL Liberase for 35 min with intermediate trituration. Cells were passed through a 40 μm nylon cell strainer, washed with washing buffer (1×PBS, 2% BSA, 0.1% sodium azide, 0.05% EDTA), and red blood cells were lysed in RBC lysis buffer. Cell counts were determined using a Countess. 1×10⁶ cells were resuspended in 1 mL DPBS and provided to the High Throughput Genomics Core for library prep. Single cell libraries were generated with Next GEM Single Cell 3′ Gene Expression Library prep v3.1 with UDI, according to manufacturer's protocol (10× Genomics PN-1000128). Sequencing libraries were applied to a NovaSeq flow cell using XP chemistry workflow v1.5 (Illumina 20043131). A 150×150 cycle paired end sequence run was performed using an Illumina NovaSeq 6000 instrument and NovaSeq S4 reagent Kit v1.5 (Illumina 20028312). Libraries were sequenced to >200 million reads per sample in a single run.

### Analysis of Single Cell RNA-Seq Data

Single cell RNA-seq data was processed with the Cell Ranger software from 10× genomics. Briefly, sequencing reads were aligned to a reference including the mm10 genome, GFP, and Cre. Feature-barcode matrices were generated using cellranger count 3.1.0 with expected-cells set to 5000.

Filtered feature matrices from cellranger were further filtered with the aid of Seurat v4.0.3. High quality cells were identified by a mitochondrial percentage <7.5 and a high number of genes/features. The feature cut-off was determined independently for each sample by the local minimum cell density between 200 and 2000 features (Foxp1 con=1000, Foxp1 CTG=1050). Each sample was run, individually, through Seurat's SCTransform pipeline, regressing out mitochondrial percentage, to generate unsupervised cell clusters. DoubletFinder v2.0.3 identified likely doublets assuming 0.8% doublets in 1000 cells, adjusted for homotypic doublets. McGinnis et al., Cell Syst. 8:329-337 e324 (2019). Doublets were excluded from downstream analysis.

Foxp1 cTG single cell samples were combined with the merge function. Clusters were manually annotated using expression of known cell markers: Vsx2, Lhx2, and Pax6 for RPCs, Neurog2 and Olig2 for neurogenic cells, Crx for photoreceptor precursors, Thrb for cones, Nrl for rods, Rbpms for retinal ganglion cells, Tfap2a for amacrine cells, and Lhx1 for horizontal cells. Differential expression analysis was performed with Seurat FindMarkers, using logfc. threshold of 0.1 and q-value <0.01.

scRNA-seq data for Jarid2 cKO was taken directly from Zhang et al., Cell Reports 42:12237 (2023) (GEO:GSE202734). Gene set enrichment analysis was run on RPC gene expression from the Jarid2 dataset and Foxp1 cTG dataset separately using gene sets obtained from the Molecular Signatures Database (Liberzon et al., 2015). Specifically, the mouse-ortholog hallmark gene sets v2022.1 were used. All detectable genes were ranked by average log 2 fold change between Jarid2 control and cKO or between Foxp1 control and cTG, and analyzed with both fgsea (v1.18.0) and gage (v2.42.0) using default settings. Gene sets were only marked as significant if they were significantly changed, in the same direction, by both methods. Q-values listed are from fgsea results.

### Quantification and Statistical Analysis

Confocal images shown are representative of at least 3 animals/6 retinas. For quantification, the number of retinal cells was quantified manually and blindly by averaging total positive cells within the central retinal region (˜300 μm length) from 4-5 sections per retina using Nikon Elements software. For EdU birth dating analysis, the number of cells colocalizing EdU with different retinal markers was counted within the central retinal region (˜1000 μm length) and averaged per retina. For cell-cycle exit analysis, EdU was injected 24 h before retina collection. Total EdU+ cells and EdU+ PCNA− cells were counted within the central retinal region (˜300 μm length) and the cell-cycle exit index was calculated as EdU+ PCNA−/total EdU+. The index was then averaged from 3-4 sections per retina.

Statistical analysis was performed in all image data using GraphPad Prism software. The sample size (n) is the number of experimental units which represents individual eyes. In each experiment, the sample size was determined to meet the criteria that statistical power would be more than 80% with the use of a two-tailed unpaired t-test at the significance level (p value) of 0.05. All graph data are presented as mean±SEM. In all graphs, dots represent individual retina. *p<0.05; **p<0.01; ***p<0.001; ****p<0.0001.

### Foxp1 Regulates the Generation of Early-Born Retinal Cell Types

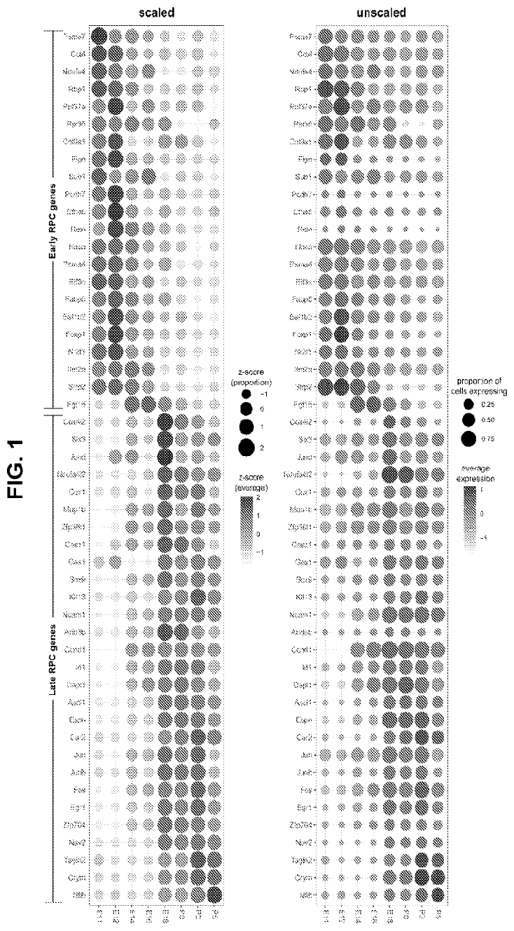

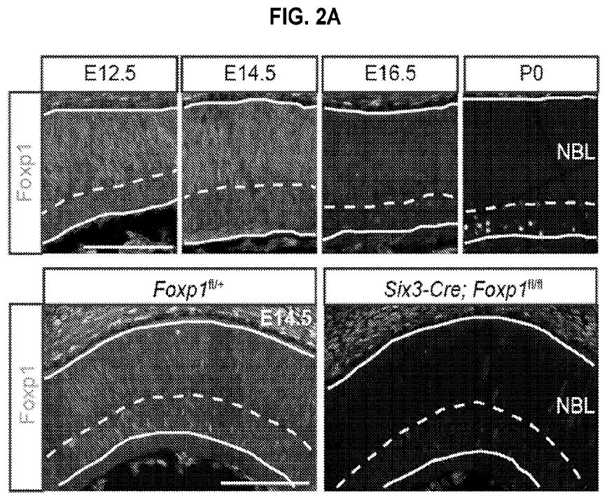

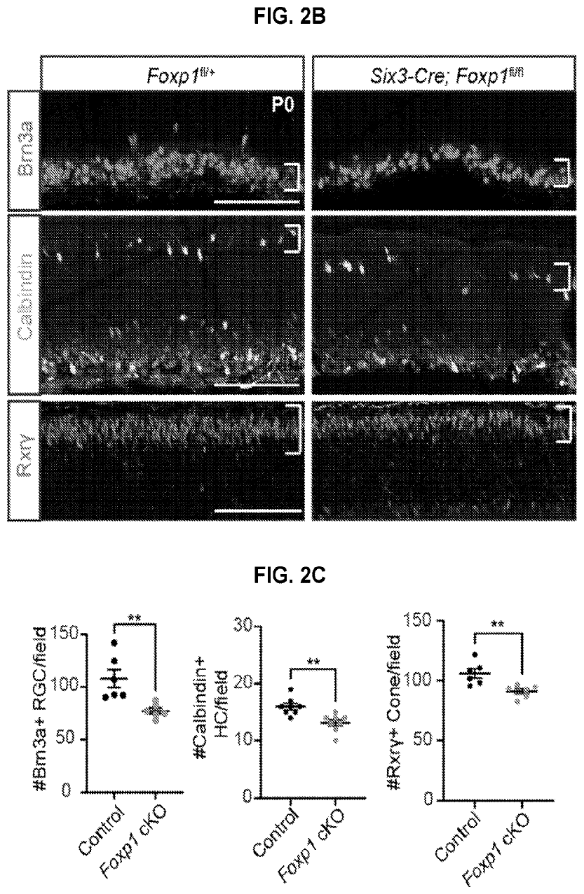

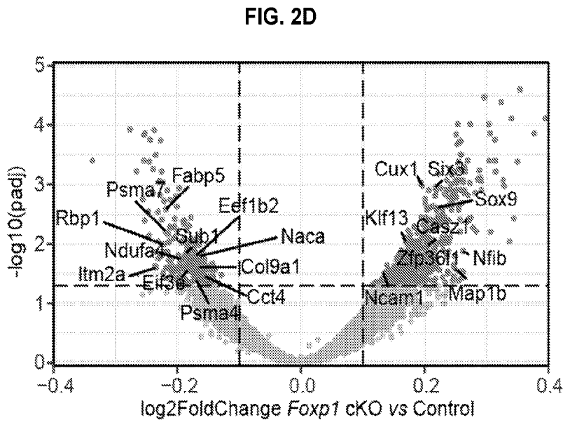

The transcription factor Foxp1 has been shown to regulate the window for early neurogenesis during cortical development and was identified as a candidate in mediating RPC competence transitions in the retina. In developing retina, Foxp1 was reported as an early RPC gene and shown to decline in expression by E17.5 (FIG. 1). To better define Foxp1 temporal expression in the developing mouse retina, antibody detection was used and these experiments showed that Foxp1 is expressed in RPCs in the neuroblastic layer at E12.5 and E14.5, with declining expression at E16.5, and virtually no detectable expression in RPCs at P0, but with strong expression in a subset of RGCs as previously reported (FIG. 2A (top row)). Thus, Foxp1 is downregulated in RPCs during the transition from early to late retinal neurogenesis.

To determine the function of Foxp1 in early RPCs, Foxp1 expression was conditionally abolished by crossing Six3-Cre mice with Foxp1-floxed mice (Foxp1 cKO) (Feng et al., 2010), and littermates lacking Cre were used as controls. The efficiency of Foxp1 cKO in the retina was confirmed by Foxp1 immunostaining at E14.5 (FIG. 2A (bottom row)). To test if the generation of early-born cell types was affected in Foxp1 cKO, immunostaining at P0 was performed for early-born retinal cell type markers. All cell types were present, as previously reported for Dkk3-Cre mediated Foxp1 cKO, so the cells were quantified to detect potential shifts in their proportions. A thinning of the ganglion cell layer (GCL) and a significant reduction in Brn3a+ RGCs (FIG. 2B-C) was observed. A significant reduction in Calbindin+ horizontal cells was also observed in the outer retina (FIG. 2B-C), and Rxrγ+ cone photoreceptors were also significantly reduced (FIG. 2B-C). Together this suggests that Foxp1 promotes early-born cell generation. To determine whether the loss of Foxp1 alters the transcriptional profile of RPCs, bulk RNA-seq was performed on E16.5 Foxp1 cKO and littermate control retinas. Several early RPC genes, including Itm2a, Ndufa4, and Col9a1, were downregulated, while a group of late RPC genes, such as Casz1, Nfib, and Sox9, were upregulated. This indicates premature expression of late RPC genes in Foxp1 cKO (FIG. 2D) suggesting that Foxp1 promotes repression of their expression. A number of these late RPC genes, including Nfib and Sox9 normally persist and function in Müller glia.

### Ectopic Foxp1 Expression in RPCs Affects Retinal Cell Genesis

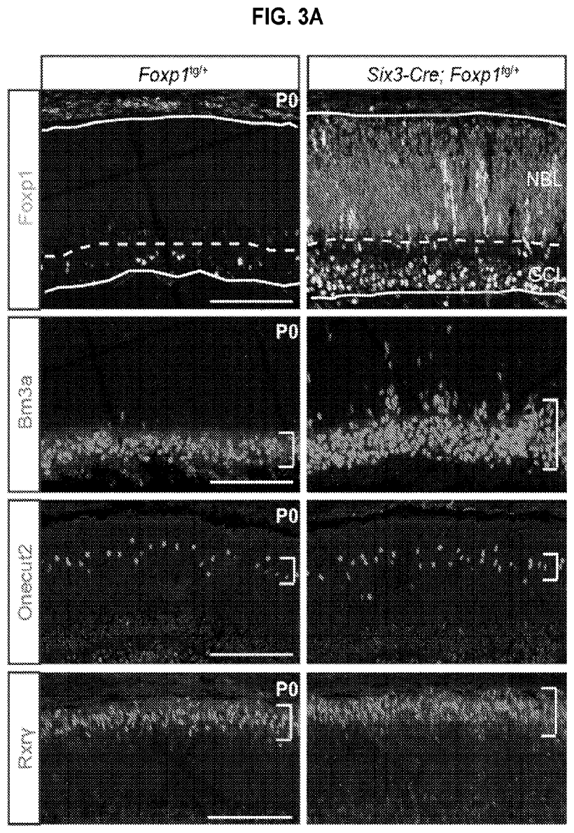

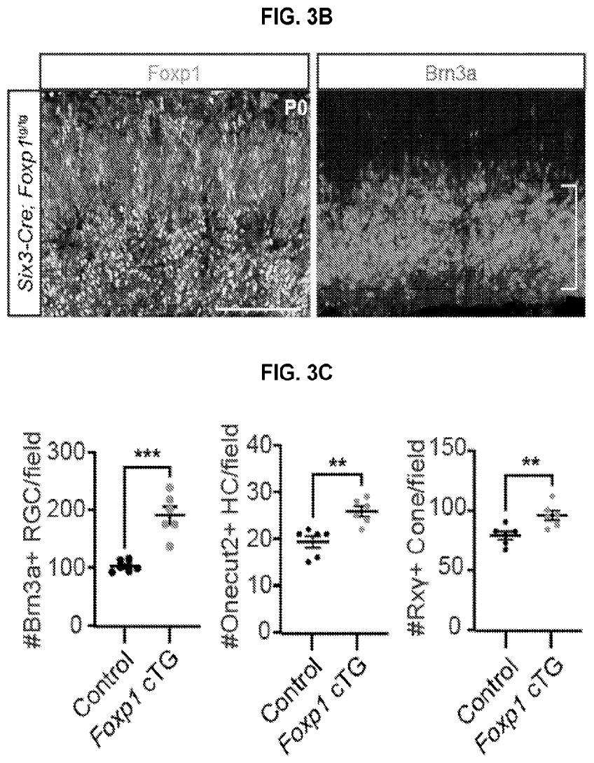

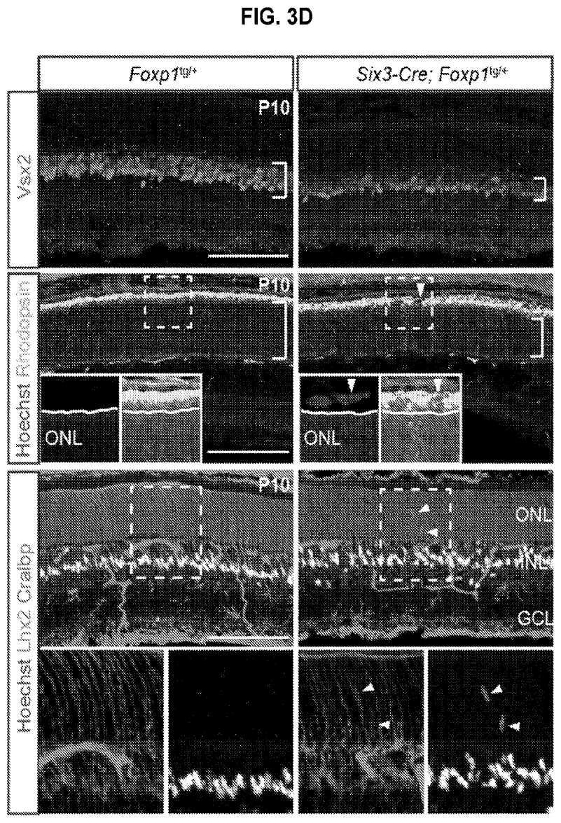

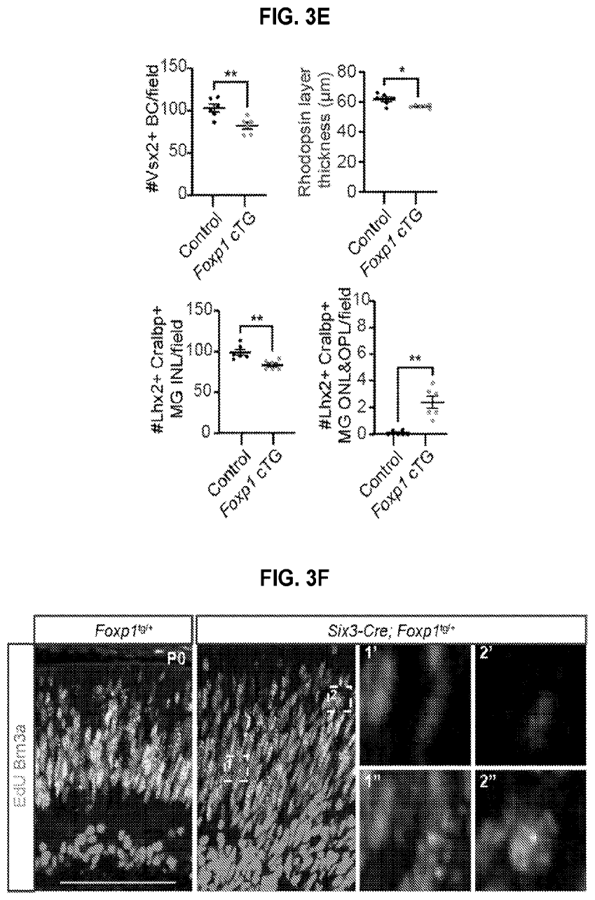

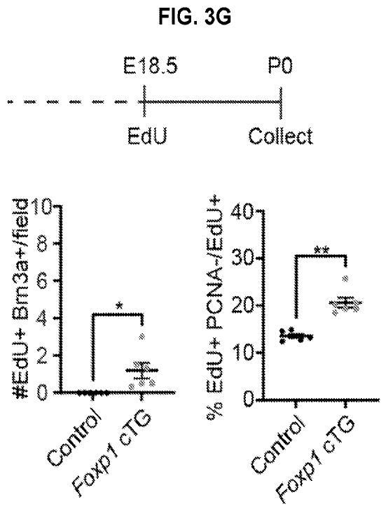

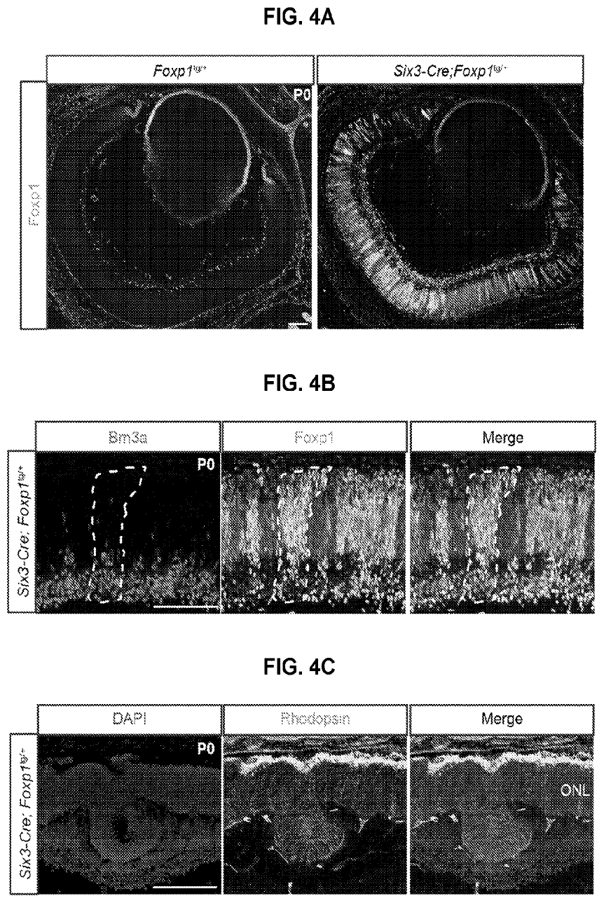

Since Foxp1 expression normally declines and is absent in RPCs by P0, experiments queried whether sustained Foxp1 expression is sufficient for the generation of early-born neurons. Foxp1 was conditionally elevated in RPCs by crossing Six3-Cre mice with conditional Foxp1 transgenic mice expressing Foxp1 (SEQ ID NO: 1) under the CAG promoter (Foxp1 cTG) (Wang et al., 2014). Immunostaining confirmed that Foxp1 expression was increased in retinal progenitors of Six3-Cre Foxp1^{tg/+} mice, with mosaic variation in levels of expression (FIG. 3A and FIG. 4A). The effects on the generation of early cell types were assessed and, by P0, overexpression of Foxp1 led to an increase in Brn3a+ RGCs in the GCL (FIGS. 3A and 12C). A significant increase in other early-born cell types was observed, including Onecut2+ horizontal cells (FIGS. 3A and 12C) and Rxrγ+ cone photoreceptors (FIGS. 3A and 12C). Brn3a+ RGCs were dramatically increased in Foxp1 cTG with two copies of the Foxp1 transgene (FIG. 3B), suggesting RGC generation is Foxp1 dosage dependent.

Additional experiments assessed whether there was a corresponding effect on late born cell generation, by examining Foxp1 cTG retina at P10. A reduction of late-born retinal cells was observed including Vsx2+ bipolar cells (FIG. 3D-E) and Rhodopsin+ rod photoreceptors (FIG. 3D-E). This is consistent with a prior study showing increased cones and reduced rods following Foxp1 overexpression. Disruption of the outer limited membrane was observed with abnormal Rhodopsin+ cells in the subretinal space (FIG. 3D) in Foxp1 cTG, although the severity of such aberrant structures was variable across sections and animals (FIG. 4B). Lhx2+ and Cralbp+ Müller glia were reduced in the inner nuclear layer (FIG. 3D-E) and some were mislocated to the outer plexiform layer and outer nuclear layer (FIG. 3E). Thus late-born cell generation is reduced and Müller glia development impacted by misexpression of Foxp1.

To determine if the increased generation of early-born retinal cells was due to an extended window of production, EdU was administered to Foxp1 cTG mice at E18.5 and immunostaining was performed for Brn3a+ RGCs at P0. Control retinas showed no Brn3a+ RGCs colabeled with EdU, in contrast to Foxp1 cTG retinas (FIG. 3F-G) indicating an extended window of RGC genesis. This extended window was coupled with enhanced exit of RPCs, as the percentage of EdU+ PCNA−/EdU+ cells was increased (FIG. 3G). Taken together, these results suggest that Foxp1 promotes the generation of early-born retinal cells by extending the window of early cell genesis at the expense of the generation of late-born cells, including Müller glia.

### Sustaining Foxp1 in RPCs Maintains Early RPC Transcription Pattern

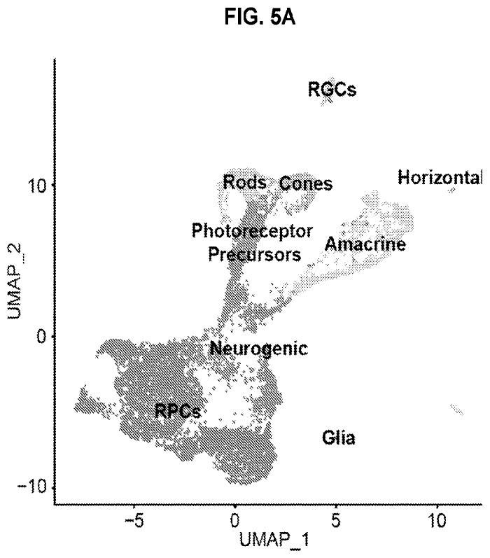

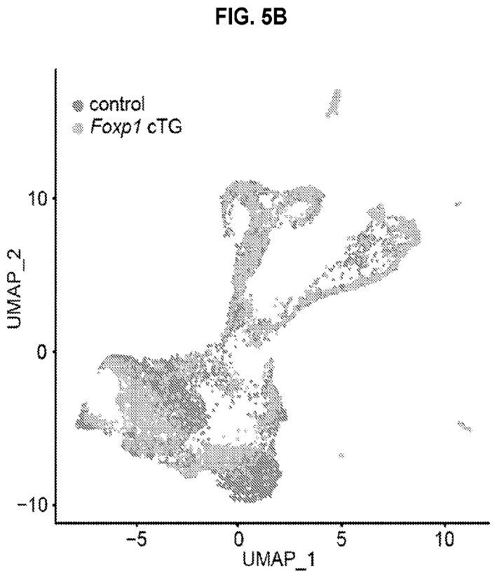

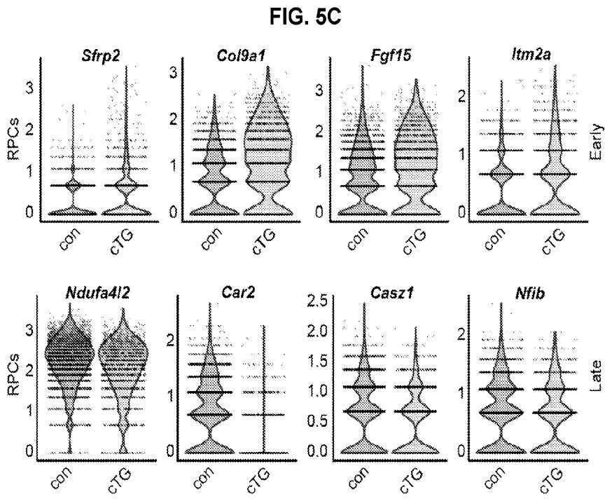

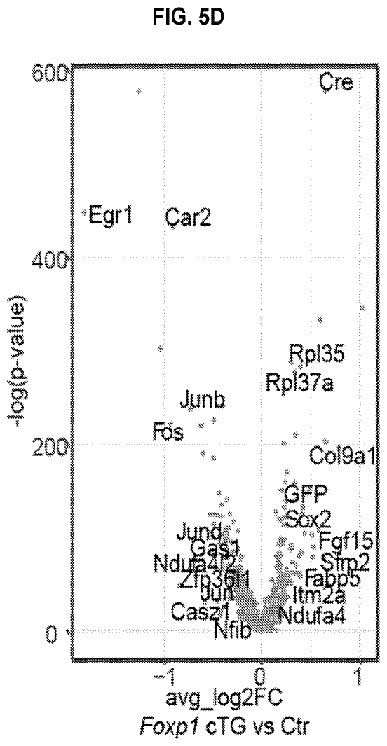

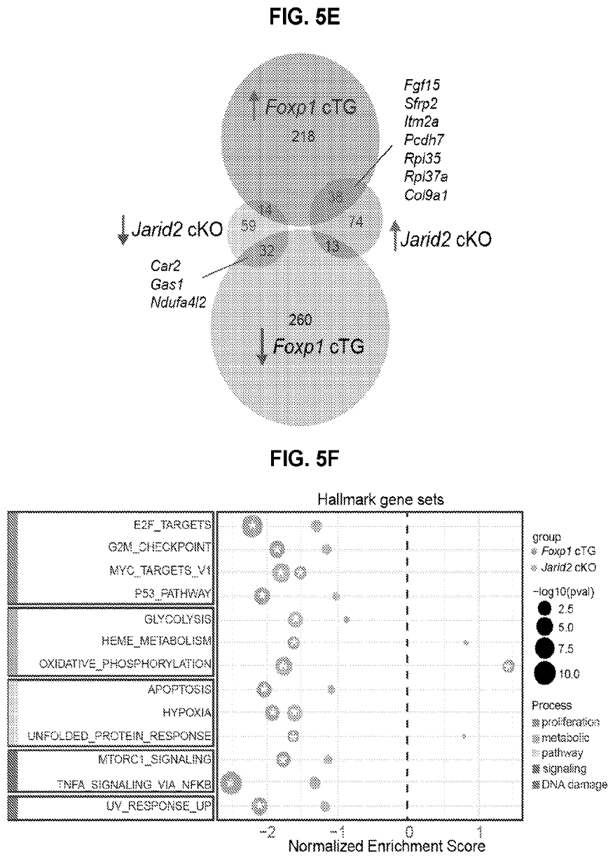

To assess if Foxp1 overexpression in RPCs changes the gene expression pattern of RPCs, scRNA-seq was performed on P0 Foxp1 cTG and control littermate retinas (FIG. 5A). A dramatic difference was observed in the UMAP embedding of RPCs between Foxp1 cTG and control retina (FIG. 5B). Focusing the analysis on RPCs, 575 differentially expressed genes were observed in the Foxp1 cTG retina. Upregulation of early RPC genes was observed, including Sfrp2, Col9a1, Fgf15, and Itm2a, and downregulation of late RPC genes Ndufa412, Car2, Casz1, and Nfib was observed, suggesting that an early RPC transcriptional pattern is artificially maintained in Foxp1 cTG RPCs (FIG. 5C-D, Table 2), and that genes characteristic of late RPCs and Müller glia are suppressed.

TABLE 2
Differential Gene Expression in Foxp1 cTG RPCs

| | | Average log2 | | | |
|---|---|---|---|---|---|
| Gene | P-value | Fold Change | Pct.1 | Pct.2 | Adjusted P-value |
| Gm47283 | 2.32 × 10⁻²⁸³ | −1.350840195 | 0.696 | 0.957 | 4.22 × 10⁻²⁷⁹ |
| Cre | 3.72 × 10⁻²⁵⁸ | 0.890683903 | 0.393 | 0 | 6.78 × 10⁻²⁵⁴ |
| Egr1 | 4.67 × 10⁻¹⁹³ | −2.059898884 | 0.155 | 0.614 | 8.51 × 10⁻¹⁸⁹ |
| Car2 | 4.85 × 10⁻¹⁸⁵ | −1.12603784 | 0.202 | 0.673 | 8.84 × 10⁻¹⁸¹ |
| Dbi | 4.25 × 10⁻¹⁴⁷ | 1.051640347 | 0.979 | 0.955 | 7.75 × 10⁻¹⁴³ |
| Tmsb10 | 8.79 × 10⁻¹⁴⁵ | 0.617061096 | 0.995 | 0.981 | 1.60 × 10−¹⁴⁰ |
| Cacna1i | 7.92 × 10⁻¹³⁰ | 0.411503904 | 0.212 | 0 | 1.44 × 10⁻¹²⁵ |
| ler2 | 1.87 × 10⁻¹²⁹ | −1.148143178 | 0.544 | 0.764 | 3.41 × 10⁻¹²⁵ |
| Rpl35 | 5.46 × 10⁻¹¹⁵ | 0.397076655 | 1 | 0.998 | 9.94 × 10⁻¹¹¹ |
| Rpl37a | 1.70 × 10⁻¹¹¹ | 0.338790481 | 1 | 1 | 3.09 × 10⁻¹⁰⁷ |
| Actb | 2.30 × 10⁻¹⁰⁹ | −0.398965019 | 1 | 1 | 4.18 × 10⁻¹⁰⁵ |
| Junb | 3.86 × 10⁻¹⁰¹ | −0.91773906 | 0.126 | 0.438 | 7.02 × 10−97 |
| Actg1 | 7.91 × 10⁻⁹⁶ | −0.492796586 | 0.995 | 0.995 | 1.44 × 10⁻⁹¹ |
| Fos | 1.27 × 10⁻⁹⁵ | −1.124633344 | 0.178 | 0.485 | 2.32 × 10⁻⁹¹ |
| Sertad1 | 4.65 × 10⁻⁹⁵ | −0.806243423 | 0.156 | 0.461 | 8.46 × 10⁻⁹¹ |
| Tubb2b | 2.95 × 10⁻⁹² | 0.734957786 | 0.84 | 0.643 | 5.37 × 10⁻⁸⁸ |
| Gm28528 | 5.83 × 10⁻⁹¹ | 0.318466617 | 0.164 | 0.004 | 1.06 × 10⁻⁸⁶ |
| Col9a1 | 3.08 × 10⁻⁸³ | 0.855503825 | 0.812 | 0.648 | 5.61 × 10⁻⁷⁹ |
| Ralgps2 | 8.83 × 10⁻⁸⁰ | −0.582235018 | 0.464 | 0.729 | 1.61 × 10⁻⁷⁵ |
| Gm42418 | 1.12 × 10⁻⁷⁷ | 0.336127799 | 1 | 1 | 2.04 × 10⁻⁷³ |
| Snhg1 | 1.81 × 10⁻⁷⁷ | −0.632540611 | 0.871 | 0.919 | 3.30 × 10⁻⁷³ |
| Rplp2 | 1.30 × 10⁻⁶⁷ | 0.255074957 | 0.999 | 1 | 2.36 × 10⁻⁶³ |
| Rsrp1 | 5.37 × 10⁻⁶⁷ | −0.500104246 | 0.773 | 0.88 | 9.77 × 10⁻⁶³ |
| Rpl41 | 4.14 × 10⁻⁶⁴ | 0.241443819 | 1 | 1 | 7.54 × 10⁻⁶⁰ |
| Eno1 | 4.71 × 10⁻⁶⁴ | 0.328115747 | 0.998 | 0.998 | 8.57 × 10⁻⁶⁰ |
| Tmsb4x | 6.33 × 10⁻⁶⁴ | 0.490954184 | 0.99 | 0.964 | 1.15 × 10⁻⁵⁹ |
| Pdpn | 8.56 × 10⁻⁶⁴ | 0.598511746 | 0.619 | 0.372 | 1.56 × 10⁻⁵⁹ |
| Cyp2j6 | 9.34 × 10⁻⁶³ | 0.484101687 | 0.307 | 0.105 | 1.70 × 10⁻⁵⁸ |
| Rbm3 | 8.79 × 10⁻⁶² | −0.386343769 | 0.959 | 0.979 | 1.60 × 10⁻⁵⁷ |
| Gadd45a | 1.75 × 10⁻⁶⁰ | −0.55067982 | 0.183 | 0.422 | 3.19 × 10⁻⁵⁶ |
| GFP | 1.35 × 10⁻⁵⁸ | 0.199182115 | 0.109 | 0.003 | 2.46 × 10⁻⁵⁴ |
| Rps29 | 1.71 × 10⁻⁵⁷ | 0.191390193 | 1 | 1 | 3.11 × 10⁻⁵³ |
| AY036118 | 3.87 × 10⁻⁵⁶ | 0.445386327 | 0.956 | 0.902 | 7.05 × 10⁻⁵² |
| Rps21 | 8.40 × 10⁻⁵⁶ | 0.242033709 | 0.999 | 1 | 1.53 × 10⁻⁵¹ |
| B230118H07Rik | 1.43 × 10⁻⁵⁵ | 0.477234478 | 0.803 | 0.635 | 2.60 × 10⁻⁵¹ |
| Lhx2 | 4.57 × 10⁻⁵⁴ | −0.452917714 | 0.747 | 0.862 | 8.31 × 10⁻⁵⁰ |
| Slc30a10 | 1.31 × 10⁻⁵³ | −0.459079294 | 0.088 | 0.292 | 2.39 × 10⁻⁴⁹ |
| Rps23 | 1.08 × 10⁻⁵¹ | 0.233011935 | 1 | 0.999 | 1.97 × 10⁻⁴⁷ |
| Cited2 | 1.11 × 10⁻⁵¹ | −0.61401298 | 0.457 | 0.647 | 2.02 × 10⁻⁴⁷ |
| Zfand5 | 1.15 × 10⁻⁵¹ | −0.504362476 | 0.637 | 0.773 | 2.10 × 10⁻⁴⁷ |
| Snhg5 | 3.23 × 10⁻⁵¹ | −0.564549495 | 0.446 | 0.636 | 5.89 × 10⁻⁴⁷ |
| Zfas1 | 1.67 × 10⁻⁴⁹ | −0.570245792 | 0.589 | 0.735 | 3.04 × 10⁻⁴⁵ |
| Rpl36a | 2.76 × 10⁻⁴⁹ | 0.261324791 | 0.998 | 0.996 | 5.03 × 10⁻⁴⁵ |
| Srsf7 | 3.40 × 10⁻⁴⁹ | −0.30385263 | 0.979 | 0.986 | 6.19 × 10⁻⁴⁵ |
| Rplp1 | 5.56 × 10⁻⁴⁹ | 0.226650495 | 1 | 1 | 1.01 × 10⁻⁴⁴ |
| Tpd52 | 2.94 × 10⁻⁴⁸ | −0.42754619 | 0.566 | 0.744 | 5.35 × 10⁻⁴⁴ |
| Csrp2 | 4.27 × 10⁻⁴⁸ | −0.358523074 | 0.94 | 0.965 | 7.78 × 10⁻⁴⁴ |
| Ifrd1 | 5.04 × 10⁻⁴⁷ | −0.48495039 | 0.508 | 0.685 | 9.17 × 10⁻⁴³ |
| Srsf3 | 1.00 × 10⁻⁴⁶ | −0.245593946 | 0.997 | 0.995 | 1.83 × 10⁻⁴² |
| Marcks | 1.75 × 10⁻⁴⁶ | 0.251229621 | 0.999 | 1 | 3.18 × 10⁻⁴² |
| Ptn | 1.82 × 10⁻⁴⁶ | 0.625299853 | 0.86 | 0.751 | 3.31 × 10⁻⁴² |
| Basp1 | 1.86 × 10⁻⁴⁶ | −0.393980756 | 0.867 | 0.925 | 3.39 × 10⁻⁴² |
| Hnrnpdl | 2.62 × 10⁻⁴⁶ | −0.362052236 | 0.881 | 0.935 | 4.77 × 10⁻⁴² |
| Nnat | 4.84 × 10⁻⁴⁶ | 0.580932211 | 0.77 | 0.624 | 8.82 × 10⁻⁴² |
| Rps27 | 9.19 × 10⁻⁴⁶ | 0.232068922 | 1 | 1 | 1.67 × 10⁻⁴¹ |
| Rps24 | 1.11 × 10⁻⁴⁵ | 0.176776121 | 1 | 1 | 2.02 × 10⁻⁴¹ |
| Gng2 | 1.31 × 10⁻⁴⁵ | 0.326646309 | 0.265 | 0.098 | 2.38 × 10⁻⁴¹ |
| H2afz | 1.39 × 10⁻⁴⁵ | −0.318079896 | 0.999 | 1 | 2.53 × 10⁻⁴¹ |
| Snhg12 | 1.86 × 10⁻⁴⁴ | −0.569869701 | 0.646 | 0.764 | 3.39 × 10⁻⁴⁰ |
| Ddit3 | 1.47 × 10⁻⁴² | −0.48477568 | 0.269 | 0.467 | 2.67 × 10⁻³⁸ |
| Vim | 2.04 × 10⁻⁴² | 0.458557863 | 0.924 | 0.876 | 3.71 × 10⁻³⁸ |
| Ubc | 1.10 × 10⁻⁴¹ | −0.375909828 | 0.852 | 0.91 | 2.00 × 10⁻³⁷ |
| Hist1h2bl | 1.33 × 10⁻⁴¹ | 0.397032542 | 0.245 | 0.094 | 2.42 × 10⁻³⁷ |
| 5430416N02Rik | 5.14 × 10⁻⁴¹ | −0.455601412 | 0.444 | 0.615 | 9.36 × 10⁻³⁷ |
| 2410006H16Rik | 1.71 × 10⁻⁴⁰ | −0.475765387 | 0.826 | 0.883 | 3.11 × 10⁻³⁶ |
| Selenow | 2.41 × 10⁻⁴⁰ | 0.328228844 | 0.964 | 0.92 | 4.39 × 10⁻³⁶ |
| Rps28 | 1.25 × 10⁻³⁸ | 0.221339486 | 0.999 | 0.998 | 2.27 × 10⁻³⁴ |
| Gm3764 | 3.31 × 10⁻³⁸ | 0.206232849 | 0.125 | 0.024 | 6.02 × 10⁻³⁴ |
| Rpl39 | 4.55 × 10⁻³⁸ | 0.195978824 | 0.999 | 0.999 | 8.28 × 10⁻³⁴ |
| Chchd2 | 1.12 × 10⁻³⁷ | −0.247153829 | 0.993 | 0.99 | 2.03 × 10⁻³³ |
| Hes5 | 1.57 × 10⁻³⁷ | 0.560717955 | 0.766 | 0.629 | 2.86 × 10⁻³³ |
| Rpl35a | 2.29 × 10⁻³⁷ | 0.187885795 | 0.999 | 1 | 4.17 × 10⁻³³ |
| Slc1a3 | 3.04 × 10⁻³⁷ | 0.453546918 | 0.828 | 0.724 | 5.53 × 10⁻³³ |
| E130114P18Rik | 1.00 × 10⁻³⁶ | −0.452628106 | 0.454 | 0.615 | 1.82 × 10⁻³² |
| Taf1d | 2.76 × 10⁻³⁶ | −0.382944262 | 0.789 | 0.857 | 5.03 × 10⁻³² |
| Eif5 | 6.95 × 10⁻³⁶ | −0.306213432 | 0.924 | 0.942 | 1.27 × 10⁻³¹ |
| Rpl37 | 1.59 × 10⁻³⁵ | 0.206127325 | 0.999 | 1 | 2.90 × 10⁻³¹ |
| Rps15a | 5.12 × 10⁻³⁵ | 0.173510507 | 1 | 1 | 9.33 × 10⁻³¹ |
| Eif1 | 6.75 × 10⁻³⁵ | −0.206233136 | 0.999 | 0.999 | 1.23 × 10⁻³⁰ |
| Fgf15 | 1.53 × 10⁻³⁴ | 0.553271236 | 0.795 | 0.704 | 2.79 × 10⁻³⁰ |
| Gadd45b | 3.02 × 10⁻³⁴ | −0.457468738 | 0.156 | 0.326 | 5.51 × 10⁻³⁰ |
| Sat1 | 4.66 × 10⁻³⁴ | −0.475093279 | 0.22 | 0.397 | 8.48 × 10⁻³⁰ |
| Jund | 4.68 × 10⁻³⁴ | −0.420462364 | 0.913 | 0.928 | 8.53 × 10⁻³⁰ |
| Sox2 | 2.03 × 10⁻³³ | 0.409899353 | 0.788 | 0.666 | 3.70 × 10⁻²⁹ |
| Sfrp2 | 2.09 × 10⁻³³ | 0.771453339 | 0.45 | 0.285 | 3.81 × 10⁻²⁹ |
| Oaz1 | 5.14 × 10⁻³³ | −0.22540088 | 0.985 | 0.992 | 9.36 × 10⁻²⁹ |
| Wnt7b | 1.80 × 10⁻³² | 0.21949815 | 0.158 | 0.049 | 3.28 × 10⁻²⁸ |
| Gas1 | 2.58 × 10⁻³² | −0.421648403 | 0.547 | 0.683 | 4.69 × 10⁻²⁸ |
| Rpl26 | 6.31 × 10⁻³² | 0.175799751 | 0.999 | 1 | 1.15 × 10⁻²⁷ |
| Rpl38 | 2.19 × 10⁻³¹ | 0.188709048 | 0.999 | 1 | 3.99 × 10⁻²⁷ |
| Lrrtm1 | 1.72 × 10⁻³⁰ | 0.324784845 | 0.34 | 0.184 | 3.13 × 10⁻²⁶ |
| Hexim1 | 2.23 × 10⁻³⁰ | −0.413553651 | 0.257 | 0.416 | 4.06 × 10⁻²⁶ |
| Prdx6 | 5.29 × 10⁻³⁰ | 0.33221175 | 0.895 | 0.831 | 9.64 × 10⁻²⁶ |
| Btg2 | 5.41 × 10⁻³⁰ | −0.440765029 | 0.269 | 0.435 | 9.86 × 10⁻²⁶ |
| Rps17 | 1.61 × 10⁻²⁹ | 0.198169639 | 0.997 | 0.997 | 2.93 × 10⁻²⁵ |
| Dusp5 | 1.93 × 10⁻²⁹ | −0.310357203 | 0.111 | 0.25 | 3.51 × 10⁻²⁵ |
| Plagl1 | 3.33 × 10⁻²⁹ | 0.367826682 | 0.839 | 0.75 | 6.06 × 10⁻²⁵ |
| Rpl23 | 3.62 × 10⁻²⁹ | 0.163217642 | 1 | 1 | 6.59 × 10−²⁵ |
| Foxp2 | 8.59 × 10⁻²⁹ | −0.278648368 | 0.07 | 0.199 | 1.56 × 10⁻²⁴ |
| Mab21l1 | 1.09 × 10⁻²⁸ | 0.289741731 | 0.306 | 0.162 | 1.99 × 10⁻²⁴ |

The gene expression pattern in Foxp1 cTG RPCs is reminiscent of that in RPCs in another transgenic model: a retinal specific knockout of Jarid2 (Jarid2 cKO) (Zhang et al., Cell Reports 42:12237 (2023)). The genes that were significantly differentially expressed in these two datasets were compared. Of the 230 genes differentially expressed in Jarid2 cKO and 575 in Foxp1 cTG RPCs, 96 were differentially expressed in both conditions. Among these 96 genes are upregulated early RPC genes (Fgf15, Sfrp2, Itm2a, Pcdh7, Ppl35, Rpl27a, Col9a1) and downregulated late RPC genes (Car2, Gas1, Ndufa412) (FIG. 5E). The large overlap between these two datasets suggests that Foxp1 and Jarid2 may regulate similar biological processes.

An unbiased approach was used to define the biological processes altered in Jarid2 cKO and Foxp1 cTG RPCs. Significantly altered MSigDB mouse hallmark gene sets were identified from each dataset through preranked gene set enrichment analysis using fgsea (FIG. 5F). Two gene sets were significantly depleted in both Jarid2 cKO and Foxp1 cTG RPCs: Myc targets V1 and hypoxia. Several additional gene sets were significantly reduced in Foxp1 cTG with Jarid2 cKO data trending in the same direction: E2F targets, G2M checkpoint, P53 pathway, apoptosis, mTORC1 signaling, Tnfα signaling via NF-kB, and UV response. The only significantly discordant result was enrichment of oxidative phosphorylation in Foxp1 cTG RPCs and depletion in Jarid2 CKO RPCs. Thus, Jarid2 and Foxp1 similarly regulate gene signatures of proliferation, biological pathways, cell signaling and DNA damage.

### Postnatal Expression of Foxp1 is Sufficient to Reopen Early RPC Competence

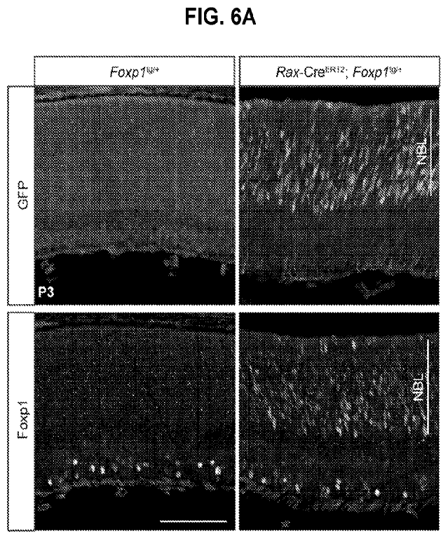

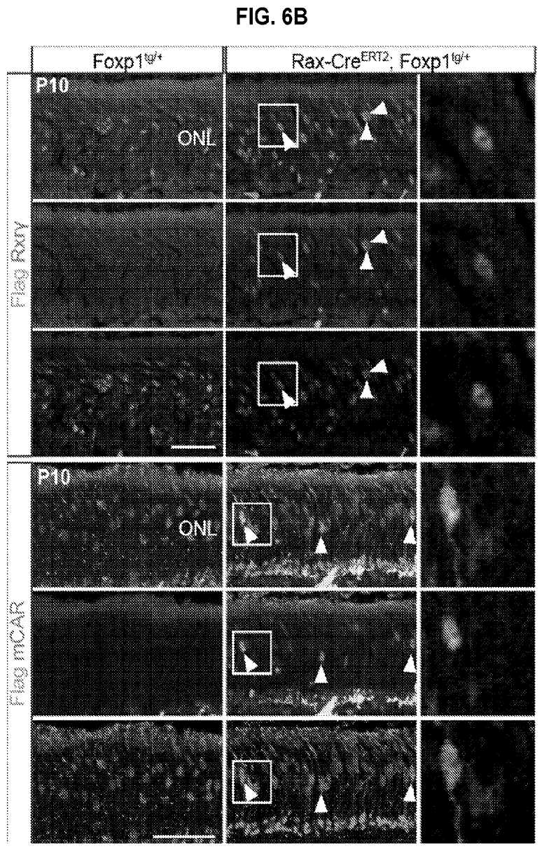

To determine whether Foxp1 acts as a factor capable of defining early RPC competence, a flag-tagged version of Foxp1 (SEQ ID NO: 1) was expressed in late RPCs of Foxp1^{tg/+} and Rax-Cre^{ERT2} Foxp119/+ littermates with tamoxifen administration at P0 and P1. Ectopic transgene expression was detected in the neuroblastic layer at P3 by immunostaining for GFP and Foxp1 (FIG. 6A). At P10, Foxp1-Flag+ Rxrγ+co-labeled cone photoreceptors were detected in Rax-Cre^{ERT2} Foxp1 cTG retina (FIG. 6B). Additionally, the mature cone marker mCAR was co-labeled with Foxp1-Flag+ cells. These results showed that re-expressing Foxp1 in late RPCs induced the generation of cone photoreceptors at the postnatal stage after the normal competence window has closed.

Temporal progression of CNS progenitors in developing retina is regulated by Foxp1, which plays a crucial role in facilitating a transition in neural progenitor competence. Foxp1 is notable, since its expression is highest at embryonic stages of retina development, and scRNA-seq analysis has identified it as a gene enriched in early RPCs. Foxp1 encodes a transcription factor of the forkhead box (FOX) family that is expressed in developing CNS, with mutations linked to neurodevelopmental disorders.

Foxp1 is a key competence factor for early retinal cell genesis. Foxp1 plays a role in early retinal cell specification, since RGCs, cones and horizontal cells were reduced in Foxp1 cKO retina. A prior study did not report a visible change in retinal neurogenesis with Dkk3-Cre mediated conditional knockout of Foxp1, potentially due to differences in Cre drivers used or stage of analysis resulting in a more subtle phenotype. They did report that overexpression of Foxp1 resulted in increased cones and reduced rods, consistent with these findings. Other related forkhead family transcription factors (Foxp2,4) are also expressed in developing retina, raising the possibility of compensation by these other factors, although Foxp1 is the most highly expressed during early development. Elevated expression of Foxp1 resulted in enhanced production of multiple early cell types and reduced generation of late cell types such as bipolar cells, rod photoreceptors, and Müller glia. This was due to an extended window for production of early cell types. This parallels the role of Foxp1 during cortex development, where its expression in apical radial glia during early cortical neurogenesis acts to gate the window for production of early born deep layer neurons. A dose-dependent effect of Foxp1 was observed, with two copies of the transgene promoting even higher levels of RGC differentiation, consistent with Foxp1 levels being highest early when RGCs are being produced then gradually declining. Notably, elevation of Foxp1 expression resulted in enhanced cell cycle exit of progenitors, which is in contrast to the effects of Foxp1 in promoting self-renewal and maintenance of apical radial glial during cortex development, suggesting potential context-dependent functions. Despite divergent effects on progenitor cell proliferation, these findings suggest a conserved role for Foxp1 in CNS progenitors in regulating the period of early neurogenesis.

RNAseq analysis at E16.5, during the period of early cell generation, revealed that loss of Foxp1 resulted in upregulation of multiple genes normally expressed in late RPCs or Müller glia, including Sox9 and Nfib. Conversely, scRNAseq analysis of Foxp1 cTG retina at P0 showed effects on RPC gene expression, with upregulation of multiple early RPC genes and downregulation of late RPC/Müller glia genes, including known late temporal identity genes Casz1 and Nfib. This raises the possibility that Foxp1 promotes extended production of early retinal cell types by repressing genes in RPCs important for late temporal identity and Müller glia development. This would be consistent with a repressor-decay timer mechanism for temporal progression whereby a temporal identity factor initiates expression when its repressor decays to a sufficiently low level, as in Drosophila. Interestingly, these findings support predictions from scATACseq analysis revealing potential cross-regulatory gene networks involving Foxp1 and Nfi. Collectively, these observations support the conclusion that expression of Foxp1 promotes early competence during retinal neurogenesis.

Importantly, reintroduction of Foxp1 expression in postnatal RPCs was sufficient to promote the generation of Rxrγ and mCAR-expressing cone photoreceptors. These results show that Foxp1 is sufficient to both extend and reopen early RPC competence to promote generation of early retinal neurons.

What is claimed:

1. A method for inducing retinal regeneration in a subject, the method comprising:
administering to the subject a therapeutically effective amount of a pharmaceutical composition comprising:
a Foxp gene expression vector comprising a polynucleotide sequence encoding a Foxp polypeptide, functional variant thereof, or fragment thereof.

2. The method of claim 1, wherein the Foxp gene expression vector is a Foxp1, Foxp2, or Foxp4 gene expression vector encoding a Foxp1, Foxp2, or Foxp4 polypeptide, functional variant thereof, or fragment thereof.

3. The method of claim 2, wherein the Foxp gene expression vector is a Foxp1 gene expression vector encoding a Foxp1 polypeptide, functional variant thereof, or fragment thereof.

4. The method of claim 1, wherein the polynucleotide sequence encoding the Foxp polypeptide, functional variant thereof, or fragment thereof has at least 90-99% identity to any one of the odd-numbered sequences from SEQ ID NO: 1-79.

5. The method of claim 1, wherein the polynucleotide sequence encoding the Foxp polypeptide, functional variant thereof, or fragment thereof is selected from any one of the odd-numbered sequences from SEQ ID NO: 1-79.

6. The method of claim 1, wherein the polynucleotide sequence encoding the Foxp polypeptide, functional variant thereof, or fragment thereof has at least 90-99% identity to any one of the odd-numbered sequences from SEQ ID NO: 25-79.

7. The method of claim 1, wherein the polynucleotide sequence encoding the Foxp polypeptide, functional variant thereof, or fragment thereof is selected from any one of the odd-numbered sequences from SEQ ID NO: 25-79.

8. The method of claim 1, wherein the Foxp polypeptide, functional variant thereof, or fragment thereof comprises an amino acid sequence having at least 90-99% identity to any one of the even-numbered sequences from SEQ ID NO: 2-80.

9. The method of claim 1, wherein the Foxp polypeptide, functional variant thereof, or fragment thereof comprises an amino acid sequence selected from any one of the even-numbered sequences from SEQ ID NO: 2-80.

10. The method of claim 1, wherein the Foxp polypeptide, functional variant thereof, or fragment thereof comprises an amino acid sequence having at least 90-99% identity to any one of the even-numbered sequences from SEQ ID NO: 26-80.

11. The method of claim 1, wherein the Foxp polypeptide, functional variant thereof, or fragment thereof comprises an amino acid sequence selected from any one of the even-numbered sequences from SEQ ID NO: 26-80.

12. The method of claim 1, wherein the pharmaceutical composition is administered to a retina of the subject by intravitreal or subretinal injection.

13. The method of claim 1, wherein the Foxp gene expression vector is selected from a viral vector, a lentiviral vector, a plasmid expression vector, an adeno-associated virus (AAV) vector, a recombinant AAV (rAAV) vector, a single-stranded AAV vector, a double-stranded AAV vector, a self-complementary AAV (scAAV) vector, or combinations thereof.

14. The method of claim 13, wherein the Foxp gene expression vector is an AAV vector of a serotype selected from AAV1, AAV2, AAV3, AAV4, AAV5, AAV6, AAV7, AAV8, AAV9, AAV10, AAV11, AAV12, or a hybrid serotype thereof.

15. The method of claim 1, wherein the Foxp gene expression vector comprises a promoter sequence operably linked to the polynucleotide sequence encoding the Foxp polypeptide, functional variant thereof, or fragment thereof.

16. The method of claim 15, wherein the promoter sequence is a retinal-specific promoter sequence or a Müller glia (MG)-specific promoter sequence.

17. The method of claim 1, wherein the pharmaceutical composition further comprises one or more nanoparticles for administration of the Foxp gene expression vector to the subject.

18. The method of claim 17, wherein the one or more nanoparticles comprise lipid-based nanoparticles, peptide-based nanoparticles, or a combination thereof.

19. The method of claim 1, wherein the Foxp polypeptide, functional variant thereof, or fragment thereof reprograms MG to generate MG-derived functional retinal neurons.

20. The method of claim 19, wherein the MG-derived functional retinal neurons comprise retinal ganglion cells and cone photoreceptors that are generated during early stages of retina development.

21. The method of claim 19, wherein the number of MG-derived functional retinal neurons in the subject is increased as compared to a baseline level of functional retinal neurons in the subject prior to administration.

22. The method of claim 1, wherein the pharmaceutical composition does not comprise a histone deacetylase (HDAC) inhibitor.

23. The method of claim 1, wherein the pharmaceutical composition does not comprise a Jak/STAT signaling pathway inhibitor.

24. The method of claim 1, wherein the subject has one or more of a retinal degenerative disease, retinal damage, or retinal blindness.

25. The method of claim 24, wherein the subject has a retinal degenerative disease comprising age-related macular degeneration (AMD), retinitis pigmentosa (RP), diabetic retinopathy (DR), central retinal artery occlusion (CRAG), vitreoretinopathy, glaucoma, Usher syndrome, optic neuropathy, optic nerve injury, or combinations thereof.

26. The method of claim 1, wherein the therapeutically effective amount of the pharmaceutical composition is administered to the subject as a single dose or as a plurality of doses.

27. A method for treating, preventing, reducing the likelihood of having, reducing the severity of, and/or slowing the progression of a retinal degenerative disease, retinal damage, or retinal blindness in a subject, the method comprising:
administering to the subject a therapeutically effective amount of a pharmaceutical composition comprising:
a Foxp gene expression vector comprising a polynucleotide sequence encoding a Foxp polypeptide, functional variant thereof, or fragment thereof.

28. The method of claim 27, wherein the Foxp gene expression vector is a Foxp1, Foxp2, or Foxp4 gene expression vector encoding a Foxp1, Foxp2, or Foxp4 polypeptide, functional variant thereof, or fragment thereof.

29. The method of claim 28, wherein the Foxp gene expression vector is a Foxp1 gene expression vector encoding a Foxp1 polypeptide, functional variant thereof, or fragment thereof.
